(19) United States
(12) Patent Application Publication
(10) Pub. No.: US 2012/0321716 A1
(43) Pub. Date: Dec. 20, 2012

(54) TECHNOLOGY FOR PREVENTING ABUSE OF SOLID DOSAGE FORMS

(76) Inventors: Michael VACHON, Quebec (CA); Edward M. RUDNIC, Potomac, MD (US)

(21) Appl. No.: 13/400,004

(22) Filed: Feb. 17, 2012

Related U.S. Application Data
(60) Provisional application No. 61/443,966, filed on Feb. 17, 2011.

(51) Int. Cl. A61K 9/00; A61P 25/04; A61K 47/32; A61K 47/18; A61K 9/14; A61K 31/485

(52) U.S. Cl. 424/490; 424/400; 514/282; 514/772.6; 514/788

(57) ABSTRACT

Abuse resistant pharmaceutical formulations are provided that contain one or more abusable drugs and one or more abuse deterrent components. The abuse deterrent component(s) prevent the abusable drug(s) from being removed/extracted to an appreciable extent and/or rate. The abuse deterrent component(s) may be in the form of pellets, beads, beadlets, granules, powders, or the like, and may comprise a core that contains a material that is both hydrophilic and hydrophobic, and optionally a pH-dependent coating.

### CROSS-REFERENCE TO RELATED APPLICATIONS

This application claims priority to U.S. provisional application Ser. No. 61/443,966, filed Feb. 17, 2011, the entirety of which is incorporated herein by reference.

### FIELD OF THE INVENTION

The present invention relates to abuse resistant pharmaceutical formulations. In certain aspects, the present invention is aimed at the deterrence of abuse and illegal attempts to remove the active agent(s) from pharmaceutical drug products that have a high rate of abuse. The present invention may include pellets, beads, beadlets, granules, powders, or the like, that are incorporated into a solid dosage form to prevent the active agent(s) from being removed to an appreciable extent and/or rate.

### BACKGROUND OF THE INVENTION

Many pharmaceutical drugs, such as those that are psychoactive or analgesic, have a significant ability to cause euphoria or pleasurable effects, and are thereby at risk for abuse. In many instances such drugs are crushed, melted, dissolved or altered; and they are then inhaled, snorted, injected or swallowed in a manner, or dosage, that is inconsistent with their safe usage. Tampering of immediate release or extended release formulations in particular will rapidly deliver a massive dose and produce a variety of serious and life threatening side effects, including respiratory depression and failure, sedation, cardiovascular collapse, coma and death.

One type of pharmaceutical drug that is particularly tampered is opioids. One common method of extracting an opioid from its dosage form is by first mixing the dosage form with a suitable liquid (e.g., water or alcohol), and then filtering and/or extracting the opioid from the mixture for intravenous injection. Another method involves dissolving extended release dosage forms of opioids in water, alcohol or another “recreational” liquid to hasten the release of the opioid, and then ingest the contents orally; this method provides high peak concentrations of the opioid in the blood, which can have a euphoric effect.

Various technologies to prevent tampering or drug abuse have been developed but each with limited success, as creative and diligent abusers with knowledge of chemistry and basic pharmaceutical techniques often learn of ways to circumvent the abuse-deterrent technology. For instance one approach consists of combining, in the same pharmaceutical formulation, the active ingredient and an agent capable of limiting the psychotropic effect of the active ingredient when the formulation is taken parenterally. This is the case, for example, with formulations combining methadone and naloxone, initially described in U.S. Pat. No. 3,966,940 and U.S. Pat. No. 3,773,955.

U.S. Pat. No. 6,696,088 describes an approach in which an opioid and an antagonist are interdispersed in a pharmaceutical formulation, such that the antagonist is “sequestered” in a form that prevents it from being released when the medicinal product is taken normally by the oral route. While the pharmaceutical formulation in this approach plays a predominant role against abuse, the necessary chemical association of the two compounds leads to a complex manufacturing process and high production costs.

U.S. Pat. No. 7,332,182 describes a pharmaceutical form in which the opioid is associated not only with an antagonist, but also with an irritant sequestered in a closed compartment. Tampering with the pharmaceutical form leads to release of the irritant. This form therefore requires the association of three active agents and the creation of compartments, which makes its manufacture complex and more costly than a simple pharmaceutical form such as a tablet.

Other companies have developed pharmaceutical systems in which the opioid or active substance is not associated with an antagonist. For example, U.S. Pat. No. 7,771,707 teaches the manufacture of an oral dosage pharmaceutical formulation in which an opioid forms a salt with one or more fatty acids, thereby increasing its lipophilicity and preventing its immediate release if the pharmaceutical form is tampered. Yet, said formulation requires chemical conversion of the active agent.

There is therefore a need for a pharmaceutical formulation that allows for the safe administration of abuse-susceptible active agents that are released over an extended period of time, i.e., which makes both crushing and dissolution of the formulation highly inefficient or even impossible, and further which prevents the extraction and separation of the active agent from the agents responsible for its sustained release. In addition, it must be possible for this pharmaceutical formulation to be produced using a relatively simple manufacturing method that is rapid and of low cost.

The present invention is aimed at the deterrence of abuse and illegal attempts to remove the active agent(s) from pharmaceutical drug products, especially those active agents that are water soluble.

### SUMMARY OF THE INVENTION

Various embodiments of the present invention relate to abuse resistant pharmaceutical formulations. In certain embodiments, the abuse resistant pharmaceutical formulations comprise a matrix having one or more abusable drugs and one or more abuse deterrent components. In some embodiments, the one or more abuse deterrent components is in the form of pellets, beads, beadlets, granules, powder, or the like, or combinations thereof. In certain embodiments, each abuse deterrent component comprises a core comprising one or more materials that are both hydrophilic and hydrophobic, which slows and/or reduces extraction of said one or more abusable drugs by aqueous or alcoholic liquids. In further embodiments, the abuse deterrent pellet, bead, etc. may also comprise a coating that does not affect the disintegration process of the solid dosage form.

In certain embodiments, the abuse resistant pharmaceutical formulation comprises one or more abusable drugs comprising amphetamines, anti-depressants, hallucinogenics, hypnotics and major tranquilizers. Examples of abusable drugs include alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propiram, propoxyphene, sufentanil, tramadol, tilidine, pharmaceutically acceptable salts thereof, prodrugs thereof, or combinations thereof.

In certain embodiments, the one or more abusable drugs may be water soluble, which include, but are not limited to, alfentanil, allylprodine, butorphanol, codeine, hydrocodone, hydromorphone, methadone, morphine, oxycodone, oxymorphone, pentazocine, tramadol and pharmaceutically acceptable salts thereof, prodrugs thereof, or combinations thereof.

In certain embodiments, the abuse resistant pharmaceutical formulation comprises one or more abusable drugs comprising morphine and oxycodone.

In some embodiments, the material that is both hydrophilic and hydrophobic comprises a viscosity increasing agent (VIA) such as polyacrylic acid, acrylic acid cross-linked with allyl ethers of polyalcohols, hydroxypropyl methylcellulose:hydroxypropyl cellulose mixture, polyvinylpyrrolidone (PVP), polyethylene oxide, methylcellulose, xanthan gum, guar gum, hydroxypropyl cellulose, polyethylene glycol, methacrylic acid copolymer, colloidal silicon dioxide, cellulose gum, starch, sodium starch glycolate, sodium alginate, or combinations thereof. In certain embodiments, the material may be a carbomer such as Carbopol®, for example, Carbopol 71G, Carbopol 971P, or Carbopol 974P.

In some embodiments, the one or more abuse deterrent components is in a ratio to the rest of the formulation of between about 1:1 w/w and about 1:5 w/w. In certain embodiments, the one or more abuse deterrent components is in a ratio to the one or more abusable drugs of between about 1:1 w/w and about 1:10 w/w.

In embodiments of the present invention, the pharmaceutical formulation may comprise one or more alkalining agents. In some embodiments, the alkalining agent(s) may be selected from the group consisting of polyplasdone XL, talc, meglumine, NaHCO₃, and PVP. In some embodiments, the alkalining agent(s) is in a form selected from the group consisting of pellets, beads, beadlets, granules, powder, or a combination thereof. In certain embodiments, the alkalizing agent(s) is in a ratio to the one or more abuse deterrent component of between about 40:60 w/w and about 80:20 w/w, or between about 60:40 w/w and about 70:30 w/w.

In yet further embodiments, the abuse resistant pharmaceutical formulation comprises a plasticizer. In some embodiments, the plasticizer is triethyl citrate.

In certain embodiments of the invention, the formulation is immediate release, controlled release, or a combination thereof.

Embodiments of the present invention relate to a method of reducing the amount of one or more abusable drugs that can be extracted by aqueous or alcoholic liquids from a pharmaceutical formulation that comprises the one or more abusable drugs. Embodiments of the present invention also relate to a method of reducing the rate at which an abusable drug can be extracted by aqueous or alcoholic liquids from a pharmaceutical formulation that comprises the one or more abusable drugs. In certain embodiments, the method comprises admixing the abusable drug(s) with one or more abuse deterrent components of the present invention. In some embodiments, the admixing occurs during preparation of the formulation.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 illustrates a pharmaceutical formulation according to some embodiments of the present invention, wherein the pharmaceutical formulation is in an immediate-release, solid oral dosage form and comprises an immediate-release abusable drug and coated abuse deterrent components.

FIG. 2 illustrates a pharmaceutical formulation according to some embodiments of the present invention, wherein the pharmaceutical formulation is in an dual-release, solid oral dosage form and comprises an immediate release component containing an abusable drug, or combination of drugs, a second delayed/modified release component containing an abusable drug, or combination of drugs, and coated abuse deterrent components.

FIG. 3 shows an image of xanthan gum (18%)-containing uncoated pellets (lot L066-01008) at magnification 25×.

FIG. 4 shows an image of Carbopol (11%)-containing uncoated pellets (lot L066-01013) at magnification 25×.

FIG. 5 shows an image of sodium alginate (36%)-containing uncoated pellets (lots L066-01015 and L066-01018) at magnification 25×.

FIG. 6 shows an image of Carbopol (12.5%)-containing uncoated pellets (lot L066-01019K) at magnification 25×.

FIG. 7 shows an image of sodium alginate (25%)/Carbopol (5%)-containing uncoated pellets (lot L066-01020B) at magnification 25×.

FIG. 8 shows an image of sodium alginate (10%)/Carbopol (10%)-containing uncoated pellets (lot L066-01020E) at magnification 25×.

FIG. 9 shows an image of sodium alginate (35%)/Carbopol (5%)-containing uncoated pellets (lot L066-01020Eb) at magnification 25×.

FIG. 10 shows an image of sodium alginate (30%)/Carbopol (5%) containing uncoated pellets (lot L066-01020H) at magnification 25×.

FIG. 11 shows an image of sodium alginate (30%)/Carbopol (1.5%)/Carbopol 974 (6.5%) containing uncoated pellets (lot L066-010201) at magnification 25×.

FIG. 12 shows an image of sodium alginate (30%)/Carbopol (5%) containing uncoated pellets (lot L066-01020H) at magnification 25×.

FIG. 13 shows an image of Carbopol (13.5%)-containing uncoated pellets (lot L066-01004A) at magnification 25×.

FIG. 14 shows filtrates resulting from extraction testing of coated Carbopol (0.3 g) and meglumine (0.2 g) pellets and a mixture of caffeine-MCC (0.5 g), using water as the extraction liquid.

FIG. 15 shows filtrates resulting from extraction testing of coated Carbopol (0.3 g) and meglumine (0.2 g) pellets and a mixture of caffeine-MCC (0.5 g), using vodka as the extraction liquid.

FIG. 16 shows filtrates resulting from extraction testing of coated Carbopol (0.6 g) and meglumine (0.4 g) pellets and a mixture of caffeine-MCC (0.5 g), using water as the extraction liquid.

FIG. 17 shows the filtration step during extraction testing of a mixture of a MCC-caffeine mixture containing 100 mg of caffeine, and Carbopol and meglumine uncoated pellets, in which 10 mL of water was used as the extraction liquid; the left image and the right image show the use of a coffee filter and a cotton ball, respectively, as filtering medium.

FIG. 18 shows filtrates from extraction testing of a mixture of a MCC-caffeine mixture containing 100 mg of caffeine, and Carbopol and meglumine pellets, in which 20 mL of water was used as the extraction liquid, and a coffee filter was used as the filtering medium.

FIG. 19 shows the filtration step during extraction testing of a mixture of a MCC-caffeine mixture containing 500 mg of caffeine, and 0.6 and 0.4 g of Carbopol and meglumine pellets, respectively; water, as the extraction liquid, was added successively in volumes of 10 mL, 10 mL, and 20 mL, and a cotton ball was used as the filtering medium (Sample 8-2).

FIG. 20 shows filtrate from extraction testing of a mixture of a MCC-caffeine mixture containing 500 mg of caffeine, and 0.6 and 0.4 g of Carbopol and meglumine pellets, respectively, in which 40 mL of water was used as the extraction liquid and a cotton ball was used as the filtering medium (Sample 9-1).

FIG. 21 shows filtrate from extraction testing of a mixture of a MCC-caffeine mixture containing 500 mg of caffeine, and 0.6 and 0.4 g of Carbopol and meglumine pellets, respectively, in which 50 mL of water was used as the extraction liquid and a cotton ball and spoon were used as the filtering medium; a spoon was used to compress the cotton ball (Sample 9-3).

FIG. 22 shows, in the left beaker, filtrate from extraction testing of a mixture of a MCC-caffeine mixture containing 500 mg of caffeine, and 0.6 and 0.4 g of Carbopol and meglumine pellets, respectively, after mixing and refiltering using double coffee filter and a cotton ball as filtering media (Samples 6-1 and 9-1 to 9-4); the right beaker contains filtrate from extraction testing of a MCC-caffeine mixture containing 500 mg of caffeine, without Carbopol or meglumine pellets (Sample 10-1).

FIG. 23 shows filtrates from extraction testing of a mixture of a MCC-caffeine mixture containing 500 mg of caffeine, and Carbopol and meglumine pellets in an amount and ratio of 0.5 g and 1.5, respectively (Samples V1-1 and V1-2); 1.0 g and 2.3, respectively (Samples V2-1 and V2-2); and 1.0 g and 1.5, respectively (Samples V3-1 and V3-2); or without Carbopol or meglumine pellets (Sample V5-1).

FIG. 24 shows filtrates from extraction testing of a mixture of a MCC-caffeine mixture containing 500 mg of caffeine, and Carbopol and meglumine pellets in an amount and ratio of 1.0 g and 2.3, respectively (Samples V4-1 and V4-2); or without Carbopol or meglumine pellets (Sample V5-1); vodka was used as the extraction liquid.

FIG. 25 shows optical microscopy images of the Life Brand™ Filter #1 at 100×.

FIG. 26 shows optical microscopy images of the Life Brand Filter #1 (wetted sample) at 100×.

FIG. 27 shows optical microscopy images of the “No Name” Filter #1 at 100×.

FIG. 28 shows 600 mg tablet from lot L066-01027.

FIG. 29 shows meglumine pellets and rods of lot L066-01028.

FIG. 30 shows Carbopol pellets, rods and dumbbell shape pellets of lot L066-01029.

FIG. 31 shows compressed immediate release tablets comprising powder Carbopol/meglumine pellets.

FIG. 32 shows powder-Carbopol/meglumine pellets formulation, 3 to 10 tablets (between 0 and 2.0 g recovered from 10 ml liquid).

FIG. 33 shows powder-Carbopol/meglumine powder formulation, 3 to 10 tablets (unfilterable from 10 to 30 ml of liquid).

FIG. 34 shows a schematic for the morphine/oxycodone controlled release tablet with abuse deterrent pellets (“CR/AD tablets”).

FIG. 35a-e shows the filtrates for filtration testing for the crushed CR/AD tablets and the OxyContin tablets using water as a liquid in volumes of (a) 10 mL, (b) 20 mL, (c) 30 mL, (d) 40 mL, and (e) 50 mL.

FIG. 36a-e shows the filtrates for filtration testing for the crushed CR/AD tablets and the OxyContin tablets using 40% ethanol as an extraction liquid in volumes of (a) 10 mL, (b) 20 mL, (c) 30 mL, (d) 40 mL, and (e) 50 mL.

FIG. 37 shows the % of morphine sulfate released after time from direct extraction with alcohol of the crushed CR/AD tablet formulation.

FIG. 38 shows the % of oxycodone HCl released after time from direct extraction with alcohol of the crushed CR/AD tablet formulation.

FIG. 39 shows the % of oxycodone HCl released after time from direct extraction with alcohol of the crushed OxyContin tablet formulation.

### DETAILED DESCRIPTION

The present invention relates to abuse-resistant pharmaceutical formulations that may reduce the amount and/or rate that abusable drugs can be extracted when the dosage form of the formulation is tampered. By reducing the amount of the extracted abusable drug, abusers may be prevented from experiencing the euphoric, pleasurable, reinforcing, rewarding, mood altering, and/or toxic effects of the agent. By reducing the extraction rate, the abuser may be deterred because of the length of time required for the extraction process.

The term “abusable drug” may refer to any active agent that is known to have the potential for abuse. An example of an abusable drug is an opioid agonist.

The term “tampered” or “tampering” may mean any manipulation by mechanical, thermal, and/or chemical means that changes the physical properties of the dosage form, e.g., to liberate the abusable drug for immediate release if it is in sustained release formulation, or to make the abusable drug available for inappropriate use such as administration by an alternate route, e.g., parenterally. The tampering can be, e.g., by means of crushing, shearing, grinding, mechanical extraction, liquid extraction, liquid immersion, combustion, heating, or any combination thereof.

The terms “abuse” such as “abusable drug abuse,” in the context of the present invention, may refer to the effects of the abusable drug: (i) in quantities or by methods and routes of administration that do not conform to standard medical practice; (ii) outside the scope of specific instructions for use provided by a qualified medical professional; (iii) outside the supervision of a qualified medical professional; (iv) outside the approved instructions on proper use provided by the drug's legal manufacturer; (v) which is not in specifically approved dosage formulations for medical use as pharmaceutical agents; (vi) where there is an intense desire for and efforts to procure same; (vii) with evidence of compulsive use; (viii) through acquisition by manipulation of the medical system, including falsification of medical history, symptom intensity, disease severity, patient identity, doctor shopping, prescription forgeries; (ix) where there is impaired control over use; (x) despite harm; (xi) by procurement from non-medical sources; (xii) by others through sale or diversion by the individual into the non-medical supply chain; and/or (xiii) for medically unapproved or unintended mood altering purposes. The drug abuse can be in the context of intermittent use, recreational use and chronic use of the abusable drug alone or in combination with other drugs.

The term “abuse resistant,” “abuse deterrent,” and “deter abuse” may be used interchangeably in the context of the present invention and may be associated with pharmaceutical formulations and methods, or aspects thereof, that resist, deter, discourage, diminish, delay and/or frustrate (i) the intentional, unintentional or accidental physical manipulation or tampering of a dosage form (e.g., crushing, shearing, grinding, chewing, dissolving, melting, needle aspiration, inhalation, insufflation, extraction by mechanical, thermal and chemical means, and/or filtration); (ii) the intentional, unintentional or accidental use or misuse of a dosage form outside the scope of specific instructions for use provided by a qualified medical professional, outside the supervision of a qualified medical professional and outside the approved instructions on proper use provided by the drug's legal manufacturer (e.g., intravenous use, intranasal use, inhalational use and oral ingestion to provide high peak concentrations); (iii) the intentional, unintentional or accidental conversion of an extended release dosage formulation of the invention into a more immediate release formulation; (iv) the intentional and iatrogenic increase in physical and psychic effects sought by recreational drug users, addicts, and patients with pain who have an addiction disorder; (v) the attempts at surreptitious administration of a dosage form to a third party (e.g., in a beverage); (vi) the attempts to procure the dosage form by manipulation of the medical system and from non-medical sources; (vii) the sale or diversion of a dosage form into the non-medical supply chain and for medically unapproved or unintended mood altering purposes; and/or (viii) the unintentional or accidental attempts at otherwise changing the physical, pharmaceutical, pharmacological and/or medical properties of the dosage form from what was intended by the manufacturer.

The abuse resistant pharmaceutical formulations of the present invention may comprise one or more abusable drugs and one or more abuse deterrent components. In some embodiments, subjecting dosage forms comprising the formulations of the present invention to abuse, such as by crushing the dosage form and using aqueous or alcoholic liquids to extract the abusable drug, may result in a gel material that is not filterable or that has a filter rate that is diminished to an appreciable extent. In certain embodiments, the mechanism of action of the VIA may involve intermolecular interactions of the VIA with the abusable drug that may prevent the abusable drug from passing through the filtration system.

Yet, when the dosage form comprising the formulations of the present invention is not subjected to abuse and is administered as intended, the abusable drug may be released from the dosage form to achieve its intended therapeutic purpose. In other words, the abuse deterrent component(s) may not actively prevent the release of the abusable drug from the dosage form. Moreover, the abuse deterrent component(s) may not impact the dissolution rate of the abusable drug from the dosage form. In some embodiments, the abuse deterrent component(s) may not negatively impact the absorption of the abusable drug from the dosage form.

Examples of abusable drugs within the present invention may include, but are not limited to: amphetamines, amphetamine salts and/or derivatives, anti-depressants, hallucinogenics, hypnotics, major tranquilizers, and opioids. Example of opioids may include, but are not limited to, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propiram, propoxyphene, sufentanil, tramadol, tilidine, pharmaceutically acceptable salts thereof, prodrugs thereof, or combinations thereof.

The abusable drugs may be water soluble, such as alfentanil, allylprodine, butorphanol, codeine, hydrocodone, hydromorphone, methadone, morphine, oxycodone, oxymorphone, pentazocine, tramadol and pharmaceutically acceptable salts thereof, prodrugs thereof, or combinations thereof.

In certain embodiments, the abuse resistant pharmaceutical formulation comprises one or more abusable drugs comprising morphine and oxycodone.

### Abuse Deterrent Component Core

The abuse deterrent component(s) may comprise a core, which may comprise a material that has both hydrophilic and hydrophobic properties, such that extraction of the abusable drug by aqueous or alcoholic means is slowed, or even prevented, to an appreciable degree. In certain embodiments, the material may be a VIA. Examples of such materials may include, but are not limited to: long-chain carboxylic acids, long-chain carboxylic acid esters, long-chain carboxylic acid alcohols, and/or combinations thereof. An example of a long-chain carboxylic acid alcohol is cetearyl alcohol.

The long chain carboxylic acids may generally contain from 6 to 30 carbon atoms and preferably contains at least 12 carbon atoms, most preferably 12 to 22 carbon atoms. In some cases this carbon chain may be fully saturated and unbranched, while others contain one or more double bonds, 3-carbon rings or hydroxyl groups. Examples of saturated straight chain acids are n-dodecanoic acid, n-tetradecanoic acid, n-hexadecanoic acid, caproic acid, caprylic acid, capric acid, lauric acid, myristic acid, palmitic acid, stearic acid, arachidic acid, behenic acid, montanic acid and melissic acid. The long chain carboxylic acids for use in the present invention may also include unsaturated monoolefinic straight chain monocarboxylic acids, which include, but are not limited to oleic acid, gadoleic acid and erucic acid. Also useful are unsaturated (polyolefinic) straight chain monocarboxyic acids. Examples of these are linoleic acid, linolenic acid, arachidonic acid and behenolic acid. Useful branched acids include, for example, diacetyl tartaric acid. Combinations of the straight chain acids are also contemplated.

Examples of long chain carboxylic acid esters include, but are not limited to, those from the group of: glyceryl monostearates; glyceryl monopalmitates; mixtures of glyceryl monostearate and glyceryl monopalmitate (Myvaplex 600, Eastman Fine Chemical Company); glyceryl monolinoleate; glyceryl monooleate; mixtures of glyceryl monopalmitate, glyceryl monostearate, glyceryl monooleate and glyceryl monolinoleate (Myverol 18-92, Eastman Fine Chemical Company); glyceryl monolinolenate; glyceryl monogadoleate; mixtures of glyceryl monopalmitate, glyceryl monostearate, glyceryl monooleate, glyceryl monolinoleate, glyceryl monolinolenate and glyceryl monogadoleate (Myverol 18-99, Eastman Fine Chemical Company); acetylated glycerides such as distilled acetylated monoglycerides (Myvacet 5-07, 7-07 and 9-45, Eastman Fine Chemical Company); mixtures of propylene glycol monoesters, distilled monoglycerides, sodium stearoyl lactylate and silicon dioxide (Myvatex TL, Eastman Fine Chemical Company); mixtures of propylene glycol monoesters, distilled monoglycerides, sodium stearoyl lactylate and silicon dioxide (Myvatex TL, Eastman Fine Chemical Company) d-alpha tocopherol polyethylene glycol 1000 succinate (Vitamin E TPGS, Eastman Chemical Company); mixtures of mono- and di-glyceride esters such as Atmul-84 (Humko Chemical Division of Witco Chemical); calcium stearoyl lactylate; ethoxylated mono- and di-glycerides; lactated mono- and diglycerides; lactylate carboxylic acid ester of glycerol and propylene glycol; lactylic esters of long chain carboxylic acids; polyglycerol esters of long chain carboxylic acids, propylene glycol mono- and di-esters of long chain carboxylic acids; sodium stearoyl lactylate; sorbitan monostearate; sorbitan monooleate; other sorbitan esters of long chain carboxylic acids; succinylated monoglycerides; stearyl monoglyceryl citrate; stearyl heptanoate; cetyl esters of waxes; stearyl octanoate; C₁₀-C₃₀ cholesterol/lavosterol esters; and sucrose long chain carboxylic acid esters. Combinations of the long chain carboxylic acid esters are also contemplated.

In certain embodiments, the VIA may be selected from the group consisting of polyacrylic acid, acrylic acid cross-linked with allyl ethers of polyalcohols, hydroxypropyl methylcellulose:hydroxypropyl cellulose mixture, PVP, polyethylene oxide, methylcellulose, xanthan gum, guar gum, hydroxypropyl cellulose, polyethylene glycol, methacrylic acid copolymer, colloidal silicon dioxide, cellulose gum, starch, sodium starch glycolate, sodium alginate, or combinations thereof. In some embodiments, the VIA may be a carbomers (Carbopol 71G, 971P and 974P), xanthan gum, sodium alginate (Keltone), Polyox, or mixtures thereof.

The materials described above may be co-formulated with a binder, such as, but not limited to, PVP, or its' derivatives, microcrystalline cellulose (Avicel, FMC Corporation), hydroxypropyl methylcellulose, hydroxyethylcellulose, hydroxypropylcellulose, and other cellulose derivatives. In some embodiments, the binder may comprise a hydrophobic oil. Examples of hydrophobic oils include, but are not limited to, a wax, oil, lipid, fatty acids, cholesterol, or triglyceride. In certain embodiments, the binder may be selected from Transcutol, PEG-400 and Cremophor (Castor Oil).

Other excipients that may be combined with the VIA include, but are not limited to, lactose, NaHCO₃, and magnesium stearate,

In certain embodiments, disintegrants or other dispersing agents will not be needed in the abuse deterrent component(s), as the inherent nature of the deconstruction effort in the extraction and abuse of these drug products will cause the materials to be crushed, mixed, and/or disintegrated.

The pellets, beads, beadlets, granules, or the like of the abuse deterrent component(s) may be prepared in multi-stage process that includes (1) blending of the dry powders, (2) wet granulation, (3) extrusion of wet mass, (4) spheronization and (5) drying, as demonstrated in the Examples.

### Coating

The pellets, beads, beadlets, granules, or the like, of the abuse deterrent component(s) may be coated with an agent that prevents the interaction of the core and the abusable drug. The coating may be pH-sensitive so as not to affect the disintegration process of tablets, or the disaggregation process of capsules or other solid dosage forms within the gut. The coated pellets, beads, beadlets, granules, or the like, may stay largely intact until they pass into the small intestines. To the extent that disintegration of the coated pellets, beads, beadlets, granules, or the like, does occur before the small intestines, it occurs to an unappreciable extent such that the absorption of the active agent is not altered.

In one embodiment, the coating comprises methacrylic acid copolymers (Eudragit L30D-55), hypromellose acetate succinate (AQOAT AS-HF), or a mixture of these two polymer systems. Other pH-sensitive coatings can be, but are not limited to, aqueous acrylic type enteric systems such as Acryl-EZE®, cellulose acetate phthalate, Eudragit L, and other phthalate salts of cellulose derivatives that are pH-sensitive. These materials can be present in concentrations from 4-40% (w/w).

In another embodiment, the coating comprises a functional coating such as a sustained- or controlled-release film coating, or a seal coating and may include Surelease, Opadry® 200, Opadry II, and Opadry Clear.

In another embodiment, the coating comprises plasticizers. An example of a plasticizer is triethyl citrate.

The coated abuse deterrent component(s) may be mixed in any type of solid oral dosage form to make a pharmaceutical formulation of an abusable drug. The abuse deterrent component(s) does not need to be in intimate contact with the abusable drug in order to function in the deterrence of abuse.

### Pharmaceutical Formulations and Methods of Preparing

The pharmaceutical formulations for oral administration may be administered in solid dosage forms such as tablets, troches, capsules, or the like. Each dosage form may be presented as discrete units such as capsules, sachets or tablets, in which each contains a predetermined amount of each abusable drug(s) in, for example, powder or granular form, and one or more of the abuse deterrent components. Such formulations may be prepared by any of the methods of pharmacy but all methods include the step of bringing together each of the abuse drug(s) and abuse deterrent component(s) with a pharmaceutically acceptable carrier. In general, the formulations are prepared by uniformly and intimately admixing the abusable drug(s) and abuse deterrent component(s), with finely divided solid carriers and then, if necessary, shaping the product into the desired presentation. In certain embodiments, the abuse deterrent component(s) is distributed uniformly/homogeneously throughout the formulation.

As used herein the language “pharmaceutically acceptable carrier” is intended to include any and all liquids, dispersion media, coatings, antibacterial and antifungal agents, isotonic and absorption delaying agents, and the like, compatible with pharmaceutical administration. The use of such media and agents together with pharmaceutical active substances is well known in the art. These carriers may include, by way of example and not limitation, sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water. Supplementary active agents may also be incorporated into the formulations. Oral formulations generally may include an inert diluent or an edible carrier.

Pharmaceutically compatible binding agents, and/or adjuvant materials may be included as part of the formulation. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In certain embodiments, the one or more abuse deterrent components may be in a ratio to the rest of the formulation of between about 1:1 w/w and about 1:10 w/w, or between about 1:1 w/w and about 1:5 w/w. In certain embodiments, the one or more abuse deterrent components may be in a ratio to the rest of the formulation excluding the one or more abusable drugs of between about 1:1 w/w and about 1:5 w/w. In certain embodiments, the one or more abuse deterrent components is in a ratio to the one or more abusable drugs of between about 1:1 w/w and about 1:10 w/w, or between about 1:1 w/w and about 1:8 w/w.

In some embodiments, the formulations may comprise one or more alkalining agents. Alkalining agents include, but are not limited to polyplasdone XL, talc, meglumine, NaHCO₃, and PVP. The alkalizing agents may be in the form of a pellet, bead, beadlet, granule, powder, or the like, and may be coated as described above. In some embodiments, the alkalining agents may be present in a particular ratio (w/w) to the abuse deterrent component(s). Such ratios of the abuse deterrent(s) to the alkalining agent may be about 40:60 w/w to about 80:20 w/w, or therebetween; for example, about 40:60 w/w, or about 50:50 w/w, or about 60:40 w/w, or about 70:30 w/w, or about 80:20 w/w.

Oral dosage forms may be formulated in unit dosage forms for ease of administration and uniformity of dosage. The term “unit dosage form” as used herein refers to physically discrete units suited as unitary dosages for the patient to be treated; each unit containing a predetermined quantity of active compound calculated to produce the desired therapeutic effect in association with the required pharmaceutical carrier. The specification for the unit dosage forms of the invention may be dictated by and directly dependent on the unique characteristics of the active compound and the particular therapeutic effect to be achieved, and the limitations inherent in the art of compounding such an active compound for the treatment of individuals.

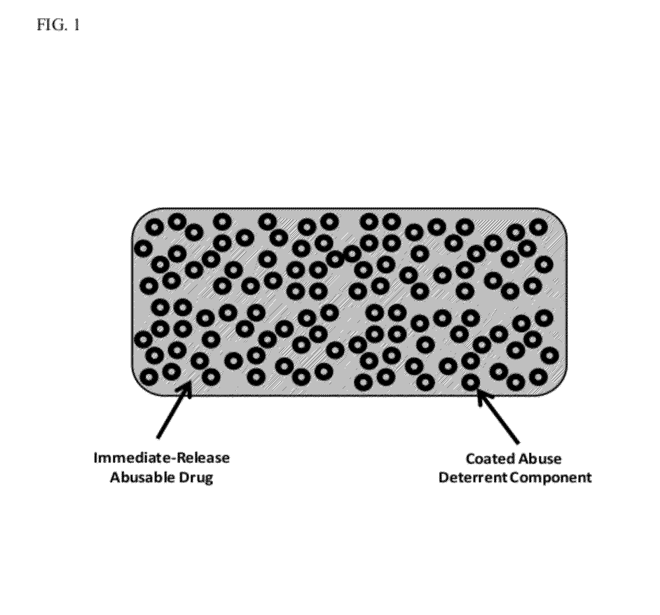

In some embodiments, the immediate release dosage form may resemble FIG. 1 in that the oral tablet may comprise an immediate release abusable drug, and coated abuse deterrent components.

The pharmaceutical formulations for oral administration may also be administered in controlled release dosage forms. For example, controlled release dosage forms as described hereinafter may be administered every 12- or 24-hours comprising, respectively, about 3 or 6 times the amount of the immediate-release dosage form. In this regard, it is well known that the change from immediate-release dosages to controlled-release dosages of opioids, such as morphine and oxycodone, can be a milligram to milligram conversion that results in the same total “around-the-clock” dose of the active agent. See Cherry and Portenoy, “Practical Issues in the Management of Cancer Pain,” in Textbook of Cancer Pain, Third Edition, Eds. Wall and Meizack, Churchill Livingstone, 1994, 1453.

Controlled-release of the active agent may be affected by incorporating the abusable drug(s) into, by way of example and not limitation, hydrophobic polymers, including acrylic resins, waxes, higher aliphatic alcohols, polylactic and polyglycolic acids and certain cellulose derivatives, such as hydroxypropyl methylcellulose. In addition, the controlled release may be affected by using other polymer matrices, liposomes and/or microspheres. The controlled release formulation of an active agent will be released at a slower rate and over a longer period of time. For example, in some embodiments in which the abusable drug(s) is one or more opioids such as morphine and/or oxycodone, the controlled release formulation may release effective amounts of a mixture of morphine and oxycodone over 12 hours. In other embodiments, the controlled release formulation may release effective amounts of morphine and oxycodone over 4 hours or over 8 hours. In still other embodiments, the controlled release formulation may release effective amounts of morphine and oxycodone over 15, 18, 24 or 30 hours.

Controlled-release formulations that may be used with the present invention include those described in U.S. patent application Ser. No. 13/024,319, filed on Feb. 9, 2011, which is incorporated herein by reference.

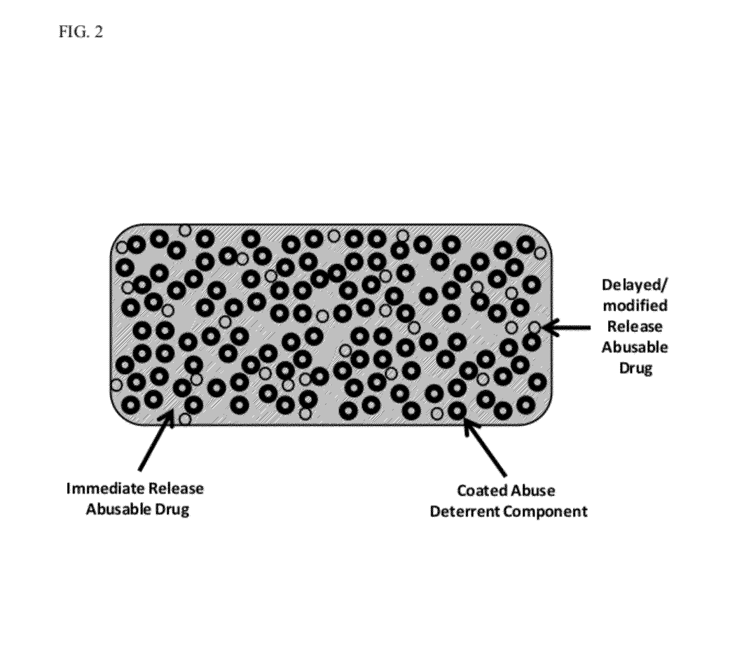

In certain embodiments, the controlled release dosage form may resemble FIG. 2 in that the oral tablet comprises an immediate release abusable drug, a delayed/modified release abusable drug, and coated abuse deterrent cores.

Whether the pharmaceutical formulation is an immediate release, controlled release, or combinations thereof, there may be over about 50, or over about 100, or over about 500, abuse deterrent components in the pharmaceutical formulation. In certain embodiments, between about 100 and about 500, or between about 500 and about 1000, coated abuse deterrent components are present in the pharmaceutical formulation.

In some embodiments, the abuse deterrent component(s) is present in the formulation in a ratio of about 1:1 w/w to the rest of the formulation, including the abusable drug(s). In other embodiments, the abuse deterrent component(s) is present in the formulation in a ratio of about 1:2 w/w, or about 1:3 w/w. or about 1:4 w/w, or about 1:5 w/w, to the rest of the formulation, including the abusable drug(s).

The abuse deterrent component(s) may be used in pre-existing pharmaceutical formulations. This ability provides a substantial advantage over the prior art abuse deterrent methods that may require a formulation change in order to incorporate the abuse deterrent system. The present abuse deterrent system does not require reformulation of an existing abusable drug formulation, which provides regulatory and cost-saving advantages.

The present invention will be understood more readily by reference to the following examples, which are provided by way of illustration and are not intended to be limiting of the invention.

### Methods of Using the Abuse Deterrent Component(s)

The abuse deterrent component(s) of the present invention may be used in a method of reducing the amount of one or more abusable drugs that can be extracted by aqueous or alcoholic liquids from a pharmaceutical formulation that comprises the one or more abusable drugs. The abuse deterrent component(s) of the present invention may also be used in a method of reducing the rate at which an abusable drug can be extracted by aqueous or alcoholic liquids from a pharmaceutical formulation that comprises the one or more abusable drugs.

These methods may comprise admixing the abusable drug(s) with one or more abuse deterrent components of the present invention. In some embodiments, the admixing occurs during preparation of the formulation.

In certain embodiments, the formulations may be pre-existing pharmaceutical formulations, such that the only formulation change is the addition of the abuse deterrent component(s).

One of ordinary skill in the art would understand how to admix the abusable drug(s) and the abuse deterrent component(s) in order to obtain a formulation with the appropriate characteristics, for example, a formulation that is uniformly mixed or homogenous.

### EXAMPLES

### Example 1

### Screening Viscosity Increasing Agents

Pharmaceutical excipients were screened for their ability to increase the viscosity of aqueous/alcoholic solutions and their potential use in abuse deterrent beadlets. Table 1 lists samples of viscosity increasing agents (VIAs) tested with or without additional excipients, and qualitative results of these agents on solution viscosity.

The screening was performed using an extraction/filtration test. Briefly, 0.5 grams of powder (or crushed tablets in the case of Sample 004) were transferred into a container and 10 mL of water (tapped water at a temperature between 26 and 28° C.) was added. The mixtures were vigorously shaken until they were homogeneous, aided by a spatula when necessary to complete homogenization. The resulting suspensions were immediately filtered through a standard coffee filter (GK Connaisseur). Viscosity increase was evaluated by visual inspection, while filtration rate was evaluated by comparing the amount of liquid added to the filter to the amount of liquid recovered in the filtrate after 10 minutes of filtration.

TABLE 1
Viscosity Increasing Agents Screening Study.

| Sample | VIA | Other Excipients | | Filtration After |
|---|---|---|---|---|
| No. | (% w/w on dry basis) | (% w/w on dry basis) | Initial Appearance | 10 minutes |
| 001 | Carbopol 71G (100%) | | Highly viscous | Unfilterable |
| | | | solution/gel | |
| 002 | Carbopol 71G (83%) | Lactose (17%) | Highly viscous | Unfilterable |
| | | | solution/gel | |
| 003 | Carbopol 71G (5%) | MCC* (54%) | Highly viscous | Unfilterable |
| | | Lactose (40%) | suspension | |
| | | NaHCO₃ (17%) | | |
| | | Mg stearate (1%) | | |
| | | (powder) | | |
| 004 | Carbopol 71G (5%) | MCC (54%) | Highly viscous | Unfilterable |
| | | Lactose (40%) | suspension | |
| | | NaHCO₃ (17%) | | |
| | | Mg stearate (1%) | | |
| | | (tablets)** | | |
| 005 | HPMC/HPC mix | | Slightly viscous | Filterable |
| | (Opadry) (100%) | | solution | |
| 006 | Plasdone K90 (100%) | | Slightly viscous | Filterable |
| | | | solution | |
| 007 | Polyox ™ (100%) | | Highly viscous | Unfilterable |
| | | | suspension | |
| 008 | Methocel E5 (100%) | | Slightly viscous | Filterable |
| | | | solution | |
| 009 | Carbopol 71G (20%) | MCC (80%) | Very viscous | Unfilterable |
| | | | suspension | |
| 010 | Carbopol 971P (20%) | MCC (80%) | Very viscous | Unfilterable |
| | | | suspension | |
| 011 | Carbopol 974P (20%) | MCC (80%) | Viscous suspension | Unfilterable |
| 012 | Polyox (20%) | MCC (80%) | Slightly viscous | Filterable |
| | | | solution | |
| 013 | Xanthan gum (20%) | MCC (80%) | Highly viscous | Unfilterable |
| | | | suspension | |
| 014 | Guar gum (20%) | MCC (80%) | Slightly viscous | Filtration rate |
| | | | suspension | decreased |
| 015 | Klucel EF (20%) | MCC (80%) | Non-viscous | Filterable |
| | | | suspension | |
| 016 | PEG 3350 (20%) | MCC (80%) | Non-viscous | Filterable |
| | | | suspension | |
| 017 | Eudragit RSPO (20%) | MCC (80%) | Slightly viscous | Filterable |
| | | | suspension | |
| 018 | Aerosil (20%) | MCC (80%) | Non-viscous | Filterable |
| | | | suspension | |
| 019 | Cellulose Gum (7L2P) | | Viscosity increases | Filtration rate |
| | (100%) | | with time | decreased with the |
| | | | | time |
| 020 | Cellulose Gum | | Viscosity increases | Filtration rate |
| | (C-5678) (100%) | | with time | decreased with the |
| | | | | time |
| 021 | HPMC AS-LG | | Non-viscous | Filterable |
| | (100%) | | suspension | |
| 022 | HPMC pH-5.0 | | Non-viscous | Filterable |
| | (100%) | | suspension | |
| 023 | Cellulose mix | | Non-viscous | Solid materials |
| | (Aquarius ®) (100%) | | stable suspension | passed through |
| | | | | the filter |
| 024 | Starch 1500 (100%) | | Slightly viscous | Filtration rate |
| | | | stable suspension | decreased |
| 025 | Sodium starch glycolate | | Slightly viscous | Filterable |
| | (100%) | | stable suspension | |
| 026 | Sodium Alginate | | Viscous cloudy | Filterable/cloudy |
| | (MANUGEL) (100%) | | liquid | liquid |
| 027 | Sodium Alginate | | Highly viscous | Unfilterable |
| | (KELTONE) (100%) | | cloudy liquid | |
| 028 | Sodium Alginate | MCC (80%) | Viscous cloudy | Filtration rate |
| | (KELTONE) (20%) | | liquid | decreased |
| 029 | Sodium Alginate | MCC (73%) | Viscous cloudy | Filtration rate |
| | (KELTONE) (18%) | Mg stearate (9%) | liquid | decreased |
| 030 | Carbopol 971P (10%) | MCC (90%) | Highly viscous | Unfilterable |
| | | | suspension | |

*MCC = microcrystalline cellulose.
**Tablets were produced from the powder blend of Sample No. 003 using a hydraulic press (Model C, Carver Inc.) with an 8 mm diameter standard concave tooling and a compression force 1750 lbf (14-15 kp).

As shown in Table 1, carbomers (Carbopol 71G, 971P and 974P), xanthan gum, sodium alginate (Keltone), Polyox, and mixtures thereof prevented the filtration using water through a coffee filter, although the results were dependent on the amount of the VIA present in the formulation. Moreover, Carbopol 71G, Carbopol 971P, Carbopol 974P, xanthan gum and sodium alginate (Keltone) either completely prevented filtration or considerably decreased filtration rate when formulations comprised 20% or less of the VIA (on a dry weight basis).

In addition, a comparison of the results of Sample No. 003 and Sample No. 004 suggest that the compression forces required for tableting does not appear to affect the anti-deterrent properties of the VIA.

### Example 2

### Process for Preparing Abuse Resistant Coated Pellets

The pellet formulations were manufactured using an extrusion/spheronization technique comprising several process stages that include: (1) blending of the dry powders, (2) wet granulation, (3) extrusion of wet mass, (4) spheronization and (5) drying, and (6) coating.

### (1) Blending of the Dry Powders

The dry ingredients were pre-mixed in a Hobart low shear mixer/granulator (model N-50) at 60 rpm for 2 minutes.

### (2) Wet Granulation

The premixed materials were wetted using a Cole-Parmer peristaltic pump to form a homogeneous wet mass suitable for the extrusion.

### (3) Extrusion of Wet Mass

The wet material was placed into a LCI Multi Granulator MG-55 extruder through the die (screen) in order to obtain cylindrical extrudates. The extruder was fitted with 1.0 mm die. Both dome and axial configurations were evaluated.

### (4) Spheronization

The extrudates were placed into a LCI Marumerizer (spheronizer) QJ-230T equipped with 2.0 mm friction plate. Spheronizer friction plate speed and time were varied according to the formulations.

### (5) Drying

Pellets were dried on trays overnight at a temperature of 50° C. (Fisher Scientific Isotemp Oven Model 655F).

### (6) Coating

The pellets were screened over an 8 inch standard sieve. After screening, the pellets with diameters below 1.0 mm and above or equal to 0.5 mm were retained for the coating process.

Pellets were first sub-coated with Opadry Clear at 5% weight gain. Opadry Clear 5% w/w solution was obtained in distilled water under stirring within 40 min. Then, an enteric coating was applied with Acryl-Eze at 10-20% in an Aeromatic Strea-1 fluid bed equipped with a Wurster column. Acryl-Eze 20% w/w suspension was obtained by dispersing the powder in distilled water according to the batch size. The suspension was stirred at room temperature for 40 min. The dispersion was screened through a 250 μm sieve prior to spraying process. The pellets were coated to a weight gain of 10-20% w/w. The pump rate was between 2 and 3 g/min, and the inlet temperature was between 38-40° C. The atomizing air pressure was between 1.0-1.4 bars. The air flow rate was controlled in order to maintain a good fluidization and outlet temperature of not more than 32° C. After spraying, air temperature was maintained for an additional 3 minutes as a final drying phase in order to avoid sticking problems.

### Example 3

### Extraction and Filtration Testing of Formulations

Pellets containing the VIAs xanthan gum, Carbopol, and sodium alginate were prepared by extrusion/spheronization and were enterically coated as described in Example 2. Table 2 provides representative pellet formulations.

TABLE 2
Coated Pellet Formulations.

| | w/w on dry basis | | | | | | |
|---|---|---|---|---|---|---|---|
| Lot | | | | Poly- | | | |
| (L066) | VIA* | MCC | Lactose | plasdone | NaHCO₃ | Talc | Granulation liquid |
| -01002 | CPL (20.0%) | 70.0% | — | 10.0% | — | — | Water: (44.0%) |
| -01003 | CPL (10.0%) | 80.0% | — | 10.0% | — | — | Water: (30.1%) |
| -01004 | CPL (13.5%) | 49.5% | 36.0% | — | — | — | Water/CaCl₂ (1%): (40.0%) |
| -01005 | CPL (20.0%) | 80.0% | — | — | — | — | EtOH anhydrous: (36.4%) |
| -01006 | XG (20.0%) | 80.0% | — | — | — | — | EtOH-Water (61:39): (73.3%) |
| -01007 | XG (16.0%) | 60.0% | 16.0% | — | — | — | EtOH-Water (50:50)/PVP |
| | | | | | | | K29/32 (8%): (50.0%) |
| -01008 | XG (18.0%) | 60.0% | 16.0% | — | — | — | EtOH-Water (45:55)/PVP |
| | | | | | | | K29/32 (6%): (85.0%) |
| -01009 | CPL (18.0%) | 60.0% | 10.0% | — | 9.0% | — | EtOH-Water (45:55)/PVP |
| | | | | | | | K29/32 (3%): (15.2%) |
| -01010 | CPL (15.0%) | 60.0% | 18.0% | — | 4.0% | — | EtOH-Water (92.7:7.3)/PVP |
| | | | | | | | K29/32 (3%): (15.2%) |
| -01011 | CPL (15.0%) | 60.0% | 18.0% | — | 5.0% | — | EtOH anhydrous/PVP K29/32 |
| | | | | | | | (2%): (38.2%) |
| -01012 | CPL (12.0%) | 60.0% | 18.0% | 4.0% | 2.0% | — | EtOH-Water (92.7:7.3)/PVP |
| | | | | | | | K29/32 (4%): (30.0%) |
| -01013 | CPL (11.0%) | 70.0% | — | — | 1.0% | 18.0% | EtOH-Water (92.7:7.3): |
| | | | | | | | (35.8%) |
| -01014 | SA (30.0%) | 60.0% | — | — | — | 10.0% | EtOH-Water (45:55): (109.6%) |
| -01015 | SA (30.0%) | 50.0% | 10.0% | — | — | 5.0% | EtOH-Water (45:55)/PVP |
| | | | | | | | K29/32 (5%) and CPL (0.6%): |
| | | | | | | | (100.0%) |
| -01016 | SA (50.0%) | 45.0% | — | — | — | — | EtOH-Water (32:68)/PVP |
| | | | | | | | K29/32 (5%): (100.0%) |
| -01017 | SA (40.0%) | 40.0% | 12.0% | — | — | — | EtOH-Water (40:60)/PVP |
| | | | | | | | K29/32 (8%): (81.0%) |
| -01018 | SA (40.0%) | 40.0% | 10.0% | — | — | — | EtOH-Water (35:65)/PVP |
| | | | | | | | K29/32 (10%): (70.0%) |

*CPL = Carbopol 971P; XG = Xanthan Gum; SA = Sodium Alginate (Keltone).

The parameters of operation for lots L066-01008, L066-01013, L066-01015 and L066-01018 where an acceptable pelletization, accompanied by a significant increasing of aqueous phase viscosity was obtained, are listed in Table 3.

TABLE 3
Extrusion/Spheronization Parameters for Lots
L066-01008, -01013, -01015, and -01018.

| | Formulation | | | |
|---|---|---|---|---|
| | -01008 | -01013 | | |
| | (Xanthan | (Carbopol | -01015 | -01018 |
| Parameters | Gum) | 971P) | (Keltone) | (Keltone) |

Dry Blending

| | | | | |
|---|---|---|---|---|
| Batch size (g) | 200 | 100 | 100 | 100 |
| Pre-mix time (min) | 3 | 5 | 4 | 3 |
| Mixer speed (rpm) | 60 | 60 | 60 | 60 |

Wet Granulation

| | | | | |
|---|---|---|---|---|
| Dose time (min:sec) | 8:44 | 2:00 | 5:00 | 3:41 |
| Total mix time | 10:00 | 2:00 | 5:30 | 4:41 |
| (min:sec) | | | | |
| Mixer speed (rpm) | 60 | 60 | 60 | 60 for 3:41 |
| | | | | 124 for 1:00 |
| Liquid rate (g/min) | 19.5 | 18.0 | 20.0 | 19.0 |
| Granulation liquid | 85.0 | 35.8 | 100.0 | 70.0 |
| (% w/w) on dry | | | | |
| basis | | | | |

Extrusion

| | | | | |
|---|---|---|---|---|
| Shaft speed (rpm) | 30 | 50 | 30 | 30 |
| Die diameter (mm) | 1.0 | 1.0 | 1.0 | 1.0 |
| | (Dome) | (Dome) | (Dome) | (Dome) |

Spheronization

| | | | | |
|---|---|---|---|---|
| Plate speed (rpm) | 1250/1000/ | 1000 | 750/500 | 500 |
| | 1750/1250 | | | |
| Spheronization | 1/6/4/2 | 8 | 1/2 | 10 |
| time (min) | | | | |

The use of xanthan gum, Carbopol, or sodium alginate for the preparation of pellets by extrusion/spheronization using pure water as granulating liquid posed a number of technological problems leading to a non-robust process. Intrinsic adhesion/stickiness of VIA materials led to poor yields, although this problem was addressed by adding some additives to the granulating liquid. While some of these approaches reduced the viscosity, using an ethanol-water mix as the granulating liquid allowed an acceptable pelletization process without affecting the physical properties of the polymers used.

Formulations containing xanthan gum (18% in lot L066-01008), Carbopol 971P (11% in lot L066-01013) and sodium alginate (30% and 40% in lots L066-01015 and L066-01018, respectively) were then produced with adequate yields for stability purposes. The pellets having size≧0.5 mm were evaluated in terms of yield (Table 4) and shape. The yields were calculated in relation to the starting powdered material. A higher level of fine materials was observed in lots L066-01015 (sodium alginate, 30%) and L066-01022 (meglumine, 20%), which represent good ranges of yields.

TABLE 4
Process Yields of Promising Formulations Containing Carbopol,
Xanthan Gum, Sodium Alginate and Meglumine.

| Lot (L066) | Yield_{≧0.5 mm} (%) |
|---|---|
| -01008 | 85.2 |
| -01013 | 74.4 |
| -01015 | 64.8 |
| -01018 | 75.3 |
| -01022* | 67.4 |
| -01023** | 85.3 |

*Lot L066-01022 contains meglumine (20%) and MCC-101 (20%).
**Lot L066-01023 contains Carbopol 971P (10%), NaHCO₃ (80%), and PVP 29/30 (5%).

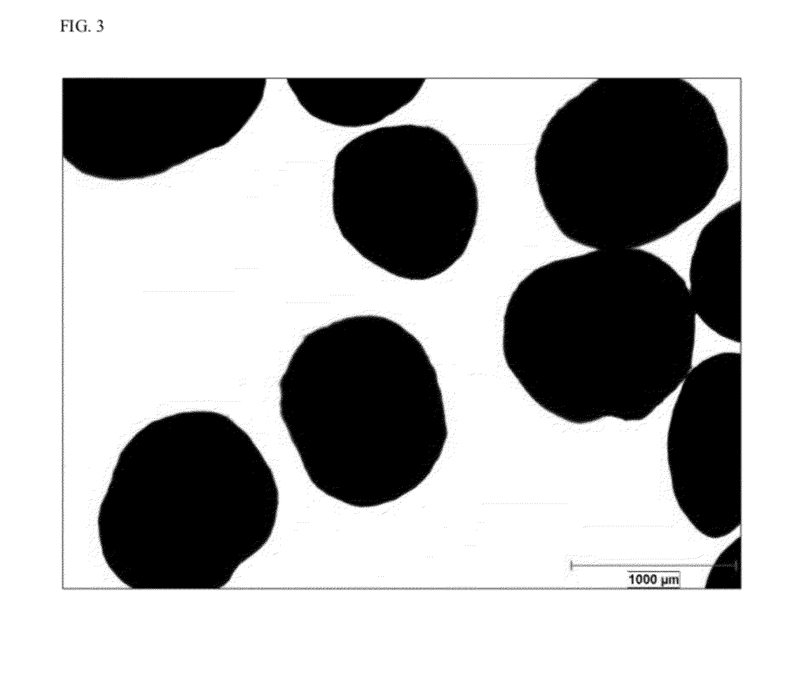

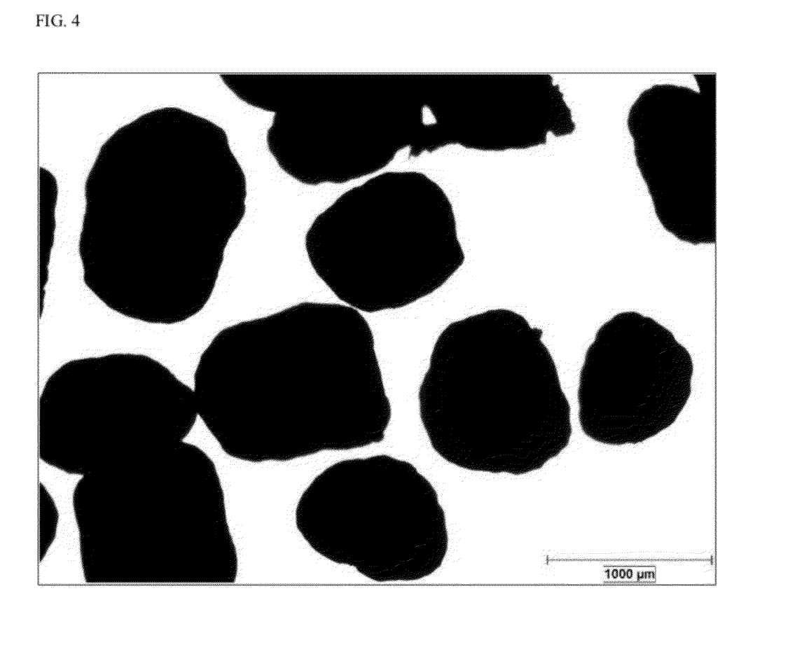

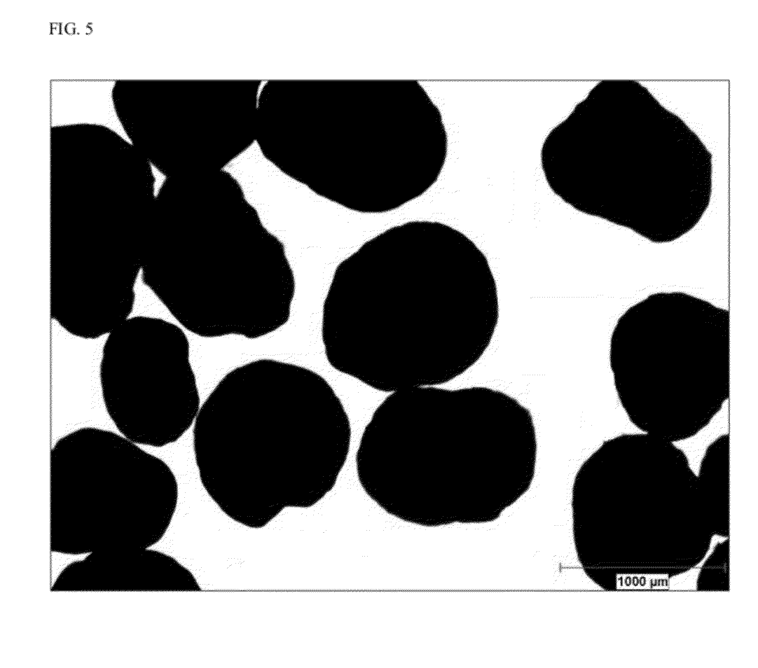

The pellets shape was assessed using a Leica DM2500 Optical Microscope under 25× magnification. Images of pellets containing 18% xanthan gum (XG), 11% Carbopol 971P (CPL) and 36% sodium alginate (SA) are shown in FIGS. 3-5, respectively. Fairly rounded shape pellets were obtained for those formulations.

### Extraction Testing

Extraction testing was performed to determine whether an active agent can be easily removed from the pellets. Caffeine was used as the active agent.

To prepare the pellets, caffeine (2 g) was dry blended with 8.0 g of MCC (MCC, Tabulose 101) using mortar and pestle. VIA-pellets that were uncoated were grinded for 15 seconds and coated pellets were grinded for 30 seconds using a hand coffee grinder (Black & Decker Home). Finally, 2.5 grams of Caffeine-MCC mix (20:80) and 2.5 grams of grinded pellets were mixed in a container with the aid of a spoon.

The extraction of caffeine from 1 g of caffeine-VIA-pellets was tested by dispersion and filtration using 10 ml of: (a) tap water, (b) vodka, (c) apple juice, (d) orange juice, and (e) 7 Up® soft drink. All these liquids were allowed to acclimate to room temperature for two hours before testing.

The caffeine-VIA-pellets were transferred into a container and the extraction liquid was added. The mixtures were vigorously shaken until homogeneous. When it was necessary, the homogenization was completed with the aid of spatula. The resulting suspensions were immediately filtered thought a coffee filter (GK Connaisseur).

As controls, 0.5 grams of caffeine-MCC mix (20:80) were transferred into a container and the extraction liquid was added and the mixtures were vigorously shaken until homogeneous. The resulting suspension was immediately filtered thought a coffee filter (GK Connaisseur). Model drug caffeine extraction using various easily available liquids from formulations containing xanthan gum, Carbopol, and sodium alginate are presented in Table 5.

TABLE 5
Extraction and Filtration of Caffeine Using Different Solvents for Control Lots
and Lots L066-01008, -01013, -01015, and -01018.*

| | | Extraction | Caffeine | VIA | | |
|---|---|---|---|---|---|---|
| | VIA-Pellet | Solvent | Conc.** | Conc.*** | Filtration Rate | Caffeine Conc. |
| Sample | Sample | (Volume mL) | (mg) | (mg/mL) | (mL/min) | in filtrate |
| Control-1 | NA | Water (10) | 10.0 | 0 | 10/0:54 | 9.4 |
| Control-2 | NA | Water (20) | 5.0 | 0 | 10/2:22 | 5.4 |
| Control-3 | NA | Vodka (10) | 10.0 | 0 | 10/2:42 | 9.9 |
| Control-4 | NA | Apple Juice (10) | 10.0 | 0 | 10/1:15 | 8.9 |
| Control-5 | NA | Orange Juice | 10.0 | 0 | 10/4:35 | 8.9 |
| | | (10) | | | | |
| Control-6 | NA | 7 Up (10) | 10.0 | 0 | 10/1:34 | 9.1 |
| -01008-1 | XG (18%) | Water (10) | 10.0 | 9.0 | 0 | NA |
| -01008-2 | XG (18%) | Water (20) | 5.0 | 4.5 | 0 | NA |
| -01008-3 | XG (18%) | Vodka (10) | 10.0 | 9.0 | 2.4/10:00 | 8.3 |
| -01008-4 | XG (18%) | Apple Juice | 10.0 | 9.0 | 3.4/10:00 | 8.6 |
| | | (10) | | | | |
| -01008-5 | XG (18%) | Orange Juice | 10.0 | 9.0 | 1.0/10:00 | 8.2 |
| | | (10) | | | | |
| -01008-6 | XG (18%) | 7 Up (10) | 10.0 | 9.0 | 0.2/10:00 | Not tested |
| -01013-1 | CPL (11%) | Water (10) | 10.0 | 5.5 | 0 | NA |
| -01013-2 | CPL (11%) | Water (20) | 5.0 | 2.8 | 0.4/10:00 | Not tested |
| -01013-3 | CPL (11%) | Vodka (10) | 10.0 | 5.5 | 0.3/10:00 | Not tested |
| -01013-4 | CPL (11%) | Apple Juice | 10.0 | 5.5 | 5.7/10:00 | 7.5 |
| | | (10) | | | | |
| -01013-5 | CPL (11%) | Orange Juice | 10.0 | 5.5 | 5.8/10:00 | 9.1 |
| | | (10) | | | | |
| -01013-6 | CPL (11%) | 7 Up (10) | 10.0 | 5.5 | 3.6/10:00 | 9.8 |
| -01015/ | SA (36%) | Water (10) | 10.0 | 18.0 | 1.7/10:00 | 0.0 |
| -01018-1**** | | | | | | |
| -01015/ | SA (36%) | Water (20) | 5.0 | 18.0 | — | NA |
| -01018-2**** | | | | | | |
| -01015/ | SA (36%) | Vodka (10) | 10.0 | 18.0 | 5.3/10:00 | 9.7 |
| -01018-3**** | | | | | | |
| -01015/ | SA (36%) | Apple Juice (10) | 10.0 | 18.0 | 0 | NA |
| -0118-4**** | | | | | | |
| -01015/ | SA (36%) | Orange Juice | 10.0 | 18.0 | 0 | NA |
| -0118-5**** | | (10) | | | | |
| -01015/ | SA (36%) | 7 Up (10) | 10.0 | 18.0 | 1.1/10:00 | 7.3 |
| -01018-6**** | | | | | | |

*5 grams of formulation were prepared: 2.5 grams of Caffeine (20%)-MCC-101 (80%) formulation + 2.5 grams of grinded enteric-coated pellets.
**Assumes that all the caffeine has dissolved.
***For example, XG: (1000 mg/g * 0.5 g of grinded pellets) * 0.18/10 mL solvent = 9.0 mg/mL.
****Lot L066-01015 and -01018 were mixed: 40.4 g of lot L066-01015 (30%) + 49.3 g of lot L066-01018 (40%) = 36% of SA.
Not tested: only the samples where 1 mL or more was recovered after 10 minutes were tested by HPLC caffeine assay;
NA: not applicable, no fluid passed through the coffee filter;
Apple juice pH = 3.5,
Orange juice pH = 3.8,
7 Up pH = 3.3.

All tested VIA pellets-formulations reduced the overall amount of drug extractable with the solvent when compared with a non-VIA formulation (controls C-1 to C-6). When 0.5 grams of xanthan gum (lot L066-01008) or Carbopol (lot L066-01013) (grinded pellets) were mixed with 0.5 grams of caffeine-MCC mix and water (10 or 20 mL), a viscous or semi-viscous aqueous liquid completely or practically unfilterable was obtained. In particular, the Carbopol 971P-based pellets considerably decreased the filtration rate from 10 mL in less than 3 minutes to 0.3 mL in 10 minutes. Xanthan gum pellets reduced the amount of filtrate using acidic juice as solvent, although it did not prevent caffeine extraction, except when 7 Up was used.

Carbopol swelling is pH dependant. In acidic media (apple juice pH=3.5, orange juice pH=3.8 and 7 Up pH=3.3), the filtration rate was only slightly decreased and all the caffeine containing formulations could be extracted. Sodium alginate (Keltone)-based pellets prevented the filtration with acidic juices, but not with vodka or 7-Up. However, using water as the solvent, a small amount of aqueous solution (1.7 mL) passed though the coffee filter, although no caffeine was found by analytical testing. That could be due to drug entrapping within the sodium alginate matrix. The resultant filtrate for this sample was a cloudy liquid with suspended particles. Prior to USP-based HPLC assay, the solutions were filtered using 5 mL BD™ syringe with a nylon membrane filter (pore size 0.45 μm).

### Example 4

### Evaluation of Granulation Liquids

The liquid vehicles shown in Table 6, which are known to be used in oral liquid formulations as solubilizers, vehicles, or absorption enhances, were tested as potential granulating liquids for the extrusion/spheronization process.

The liquid was added until an appropriate powder cohesiveness was achieved to obtain a rounded shape mass under pressure. Filtration testing was carried out immediately after preparation of the mixture.

TABLE 6
Granulating Liquids Evaluated for MCC-Carbopol Formulations.

| | % w/w on dry basis | | |
|---|---|---|---|
| Sample | Liquid vehicle* | MCC-101 | Carbopol 971P |
| L-1 | Labrasol: 50.0 | 50.0 | 0 |
| L-2 | Labrasol: 28.7 | 55.9 | 15.4 |
| L-3 | Labrafil M 1944 CS: 33.3 | 50.0 | 16.7 |
| L-4 | Labrafil M 1944 CS/ | 50.0 | 16.7 |
| | Water (80:20): 33.3 | | |
| L-5 | Transcutol P: 22.1 | 58.6 | 19.2 |
| L-6 | PEG-400: 33.6 | 53.1 | 13.3 |
| L-7 | Captex 200: 38.4 | 40.7 | 20.9 |
| L-8 | Capmul MCM: 34.5 | 49.0 | 16.5 |
| L-9 | Cremophor EL: 30.7 | 51.4 | 17.8 |

*Labrasol = caprylocaproyl macrogol-8 glycerides; Labrafil = oleoyl macrogol-6 glycerides; Transcutol = 2-(2-ethoxyethoxy)ethanol; PEG = polyethylene glycol; Captex = propylene glycol dicaprylocaprate; Capmul MCM = medium chain mono- and diglycerides; Cremophor EL = polyethoxylated castor oil.

New solvents as potential granulating liquids were evaluated in order to avoid sticking issues with water, and solvent recovery of ethanol (Table 7). The results show that Transcutol, PEG-400 and Cremophor could be employed as a liquid binder having adequate cohesiveness without affecting the properties of Carbopol as VIA in water and vodka as extraction solvents.

TABLE 7
Liquid Vehicles as Granulating Fluid for MCC-101:Carbopol 971P Formulations.

| | % w/w on dry basis | | | Results | |
|---|---|---|---|---|---|
| Sample | Liquid vehicle* | MCC | Carbopol | Cohesiveness | Filtration** |
| L-1 | Labrasol: 50.0 | 50.0 | 0 | No | Water: Filterable |
| L-2 | Labrasol: 28.7 | 55.9 | 15.4 | Yes | Water: Filterable |
| L-3 | Labrafil: 33.3 | 50.0 | 16.7 | Slight | Water: Unfilterable |
| | | | | | Vodka: Filterable |
| L-4 | Labrafil/Water (80:20): | 50.0 | 16.7 | Yes | Water: Unfilterable |
| | 33.3 | | | | Vodka: Filterable |
| L-5 | Transcutol P: 22.1 | 58.6 | 19.2 | Yes | Water: Unfilterable |
| | | | | | Vodka: Unfilterable |
| L-6 | PEG-400: 33.6 | 53.1 | 13.3 | Yes | Water: Unfilterable |
| | | | | | Vodka: Unfilterable |
| L-7 | Captex 200: 38.4 | 40.7 | 20.9 | Slight | Water: Unfilterable |
| | | | | | Vodka: Unfilterable |
| L-8 | Capmul MCM: 34.5 | 49.0 | 16.5 | Slight | Water: Unfilterable |
| | | | | | Vodka: Unfilterable |
| L-9 | Cremophor EL: 30.7 | 51.4 | 17.8 | Yes | Water: Unfilterable |
| | | | | | Vodka: Unfilterable |

*Labrasol = caprylocaproyl macrogol-8 glycerides; Labrafil = oleoyl macrogol-6 glycerides; Transcutol = 2-(2-ethoxyethoxy)ethanol; PEG = polyethylene glycol; Captex = propylene glycol dicaprylocaprate; Capmul MCM = medium chain mono- and diglycerides; Cremophor EL = polyethoxylated castor oil.
**0.5 grams of mix in 10 mL of solvent; Unfilterable: volume filtrate less than 1 mL after 10 minutes.

The different granulating liquids were further evaluated in pellet formulations prepared with Carbopol (lot L066-01019) and Carbopol/sodium alginate (lot L066-01020). For pelletization, 100 g/batch were prepared. The powdered materials were first blended for about 1 minute and the mixture was sieved using a 20 mesh sieve. The granulation liquid was slowly added into the mixture until all the material was granulated. The wet mass was immediately extruded using a LCI Multi Granulator MG-55, dome configuration with a 1.2 mm die and extrusion speed ranging from 30-50 rpm. The extrudates were spheronized at speeds between 500 and 1750 rpm for up to 20 minutes on a LCI Marumerizer QJ-230T equipped with 2.0 mm friction plate. Description of the formulations composition evaluated can be found in Table 8.

TABLE 8
Carbopol and Carbopol/Sodium Alginate Extrusion/Spheronization Formulations.

| Lot | % w/w on dry basis | | | | | |
|---|---|---|---|---|---|---|
| (L066) | IVA* | MCC-101 | Lactose | Talc | Compritol | Granulation liquid |
| -01019A | CPL: 19.0 | 59.0 | — | — | — | Transcutol: 22.0 |
| -01019B | CPL: 15.0 | 47.0 | — | — | — | Captex: 34/ |
| | | | | | | Transcutol: 4.0 |
| -01019C | CPL: 20.0 | 80.0 | — | — | — | Isopropanol 70%: |
| | | | | | | 44.5 |
| -01019D | CPL: 14.0 | 54.0 | — | — | — | PEG: 33.0/EtOH-95%: |
| | | | | | | 4.4 |
| -01019E | CPL: 20.0 | 80.0 | — | — | — | Isopropanol 99.5%: |
| | | | | | | 36.4 |
| -01019F | CPL: 15.0 | 79.0 | — | 6.0 | — | Isopropanol 99.5%: |
| | | | | | | 35.1 |
| -01019G | CPL: 12.5 | 82.5 | — | 5.0 | — | Isopropanol 99.5%: |
| | | | | | | 19.5 |
| -01019H | CPL: 15.0 | 60.0 | 25.0 | — | — | Isopropanol 99.5%: |
| | | | | | | 26.0 |
| -01019I | CPL: 12.0 | 58.0 | 30.0 | — | — | EtOH-95%: 17.3 |
| -01019J | CPL: 15.0 | 50.0 | 10.0 | 15.0 | — | Water: 5.0/EtOH-95%: |
| | | | | | | 20.0/CO: 10.0 |
| -01019K | CPL: 12.5 | 67.5 | — | 15.0 | — | EtOH-95%: 20.0/ |
| | | | | | | CO: 5.0 |
| -01020A | SA: 30.0/CPL: 5.0 | 65.0 | — | — | — | Water: 12.9/ |
| | | | | | | EtOH-95%: 19.0 |
| -01020B | SA: 25.0/CPL: 5.0 | 70.0 | — | — | — | Water: 16.0/ |
| | | | | | | EtOH-95%: 24.0 |
| -01020C | SA: 10.0/CPL: 10.0 | 70.0 | — | 2.0 | — | EtOH-95%: 16.0/ |
| | | | | | | Transcutol: 9.0 |
| -01020D | SA: 10.0/CPL: 10.0 | 55.0 | 25.0 | — | — | Water: 2.4/ |
| | | | | | | EtOH-95%: 20.3 |
| -01020E | SA: 10.0/CPL: 10.0 | 50.0 | 12.5 | 12.5 | — | EtOH-95%: CO |
| -01020Eb | SA: 35.0/CPL: 5.0 | 45.0 | 5.0 | 4.0 | — | Water: 16.7/EtOH-95%: |
| | | | | | | 40.5/CO: 6.0 |
| -01020F | SA: 35.0/CPL: 7.0 | 48.0 | — | 3.0 | — | Water: 18.8/EtOH-95%: |
| | | | | | | 39.0/CO: 7.2 |
| -01020G | SA: 35.0/CPL: 5.0 | 50.4 | — | 3.6 | — | Water: 13.0/EtOH-95%: |
| | | | | | | 30.0/CO: 6.6 |
| -01020H | SA: 30.0/CPL: 5.0 | 45.0 | 10.0 | 5.0 | — | Water: 12.5/EtOH-95%: |
| | | | | | | 12.5/CO: 5.0 |
| -01020I | SA: 30.0/CPL: 1.50/CPL974: | 42.0 | 10.0 | 4.5 | — | Water: 21.0/EtOH-95%: |
| | 6.5 | | | | | 9.0/CO: 5.4 |
| -01020J | SA: 30.0/CPL: 5.0 | 44.6 | 10.6 | — | 5.0 | Water: 15.6/EtOH-95%: |
| | | | | | | 10.4/CO: 5.0 |

*SA: Sodium alginate (Keltone); CPL: Carbopol 971P; CO: Castor oil.

For the filtration testing, the pellets were powdered using mortar and pestle. Filtration testing was done using a standard coffee filter (LIFE, Pharmaprix). 10 mL of water and vodka, were mixed with 0.5 g of powdered pellets and immediately filtrated. The recovered liquid (filtrate) was weighed after 10 minutes.

Table 9 presents the formulation trials for evaluating the effect of the granulation liquid on the process behaviour in terms of obtaining coated pellets with optimum size and shape characteristics.

TABLE 9
Carbopol and Carbopol/Sodium Alginate Formulations.

| | Formulation | | Results | |
|---|---|---|---|---|
| Lot (L066) | VIA (g) | Granulating liquid (g) | Spheronization | Filtration rate* |
| -01019A | CPL: 19.0 | Transcutol: 22.0 | Not spheronizable | NA |
| -01019B | CPL: 15.0 | Captex: 45/Transcutol: 5.0 | Not extrudable | NA |
| -01019C | CPL: 20.0 | Isopropanol 70%: 44.5 | Not extrudable | NA |
| -01019D | CPL: 14.0 | PEG: 36.6/EtOH-95%: 5.0 | Not spheronizable | NA |
| -01019E | CPL: 20.0 | Isopropanol 99.5%: 36.4 | Not spheronizable | NA |
| -01019F | CPL: 15.0 | Isopropanol 99.5%: 35.1 | Poor | Water: 3.0 g |
| | | | | Vodka: NT |
| -01019G | CPL: 12.5 | Isopropanol 99.5%: 19.5 | Poor | NT |
| -01019H | CPL: 15.0 | Isopropanol 99.5%: 26.0 | Poor | NT |
| -01019I | CPL: 12.0 | EtOH-95%: 17.3 | Poor | NT |
| -01019J | CPL: 15.0 | Water: 5.0/EtOH 95%: | Not spheronizable | NA |
| | | 20.0/CO: 10.0 | | |
| -01019K | CPL: 12.5 | EtOH-95%: 20.0/CO: 5.0 | Poor to passable | Water: 4.1 g |
| | | | | Vodka: NT |
| -01020A | SA: 30.0/CPL: 5.0 | Water: 12.9/ | Poor | NT |
| | | EtOH 95%: 19.0 | | |
| -01020B | SA: 25.0/CPL: 5.0 | Water: 16.0/ | Poor to passable | Water: 0.4 g |
| | | EtOH-95%: 24.0 | | Vodka: Filterable |
| -01020C | SA: 10.0/CPL: 10.0 | EtOH-95%: 16.0/ | Poor | Water: 3.2 g |
| | | Transcutol: 9.0 | | Vodka: NT |
| -01020D | SA: 25.0/CPL: 10.0 | Water: 2.4/ | Poor | NT |
| | | EtOH-95%: 20.3 | | |
| -01020E | SA: 10.0/CPL: 10.0 | EtOH 95%: 25.4/ | Poor to passable | Water: 1.3 g |
| | | CO: 5.0 | | Vodka: 3.6 g |
| -01020Eb | SA: 35.0/CPL: 5.0 | Water: 16.7/EtOH-95%: | Poor | Water: 0.0 g |
| | | 40.5/CO: 6.0 | | Vodka: Filterable |
| -01020F | SA: 35.0/CPL: 7.0 | Water: 18.8/EtOH-95%: | Poor | NT |
| | | 39.0/CO: 7.2 | | |
| -01020G | SA: 35.0/CPL: 5.0 | Water: 13.0/EtOH-95%: | Poor | NT |
| | | 30.0/CO: 6.6 | | |
| -01020H | SA: 30.0/CPL: 5.0 | Water: 12.5/EtOH 95%: | Poor to passable | Water: 0.0 g |
| | | 12.5/CO: 5.0 | | Vodka: Filterable |
| -01020I | SA: 30.0/CPL: 1.50/CPL974: | Water: 21.0/EtOH-95%: | Passable | Water: 2.3 g |
| | 6.5 | 9.0/CO: 5.4 | | Vodka: 0.2 g |
| -01020J | SA: 30.0/CPL: 5.0 | Water: 15.6/EtOH-95%: | Poor | Water: 1.8 g |
| | | 10.4/CO: 5.0 | | Vodka: 1.5 g |

*0.5 grams of powdered pellets, mixed with 10 mL of solvent, filtered with a coffee filter, after 10 minutes the liquid passing through the filter was weighed.
SA: Sodium alginate (Keltone),
CPL: Carbopol 971,
CO: Cremophor,
AA: meglumine as alkalining agent, ratio CPLₚₑₗₗₑₜₛ/AA = 90/10;
Filterable: all liquid pass through the filter;
NA: not applicable;
NT: not tested.

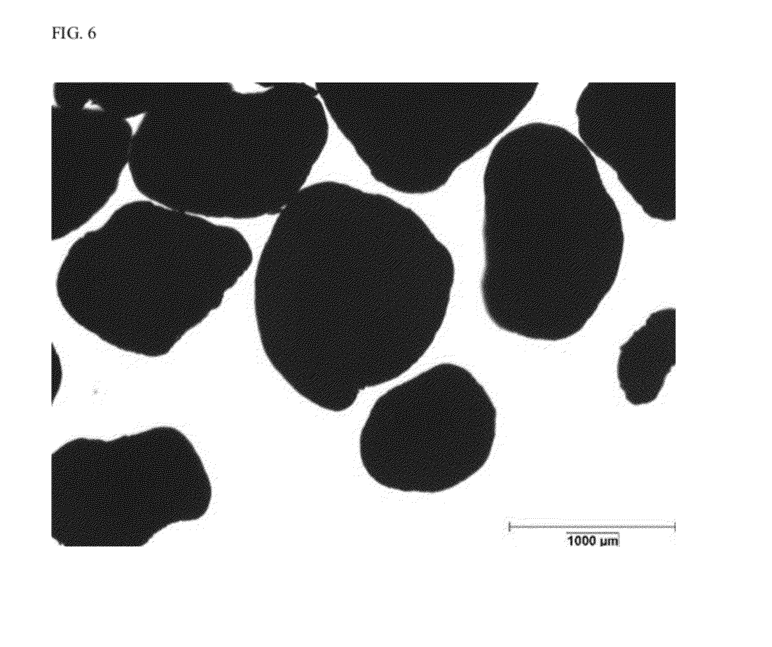

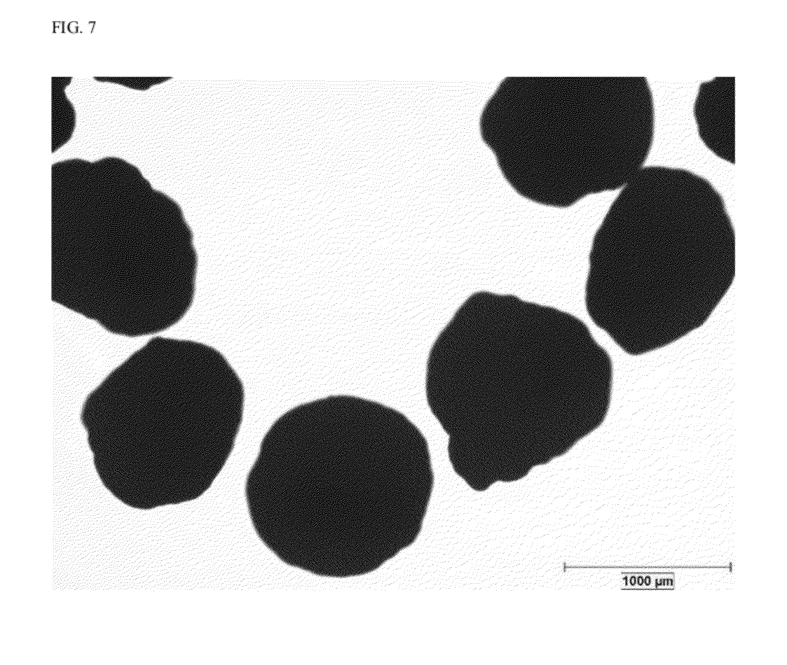

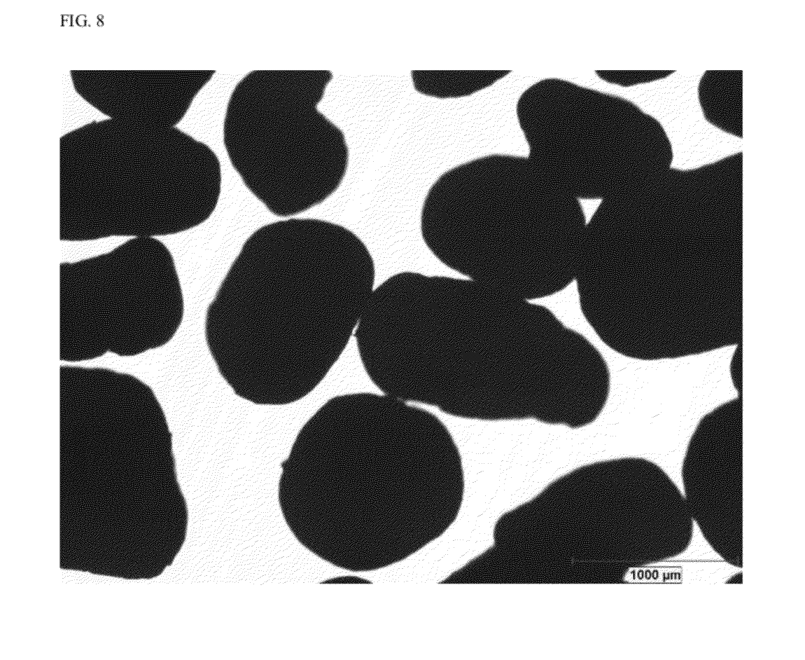

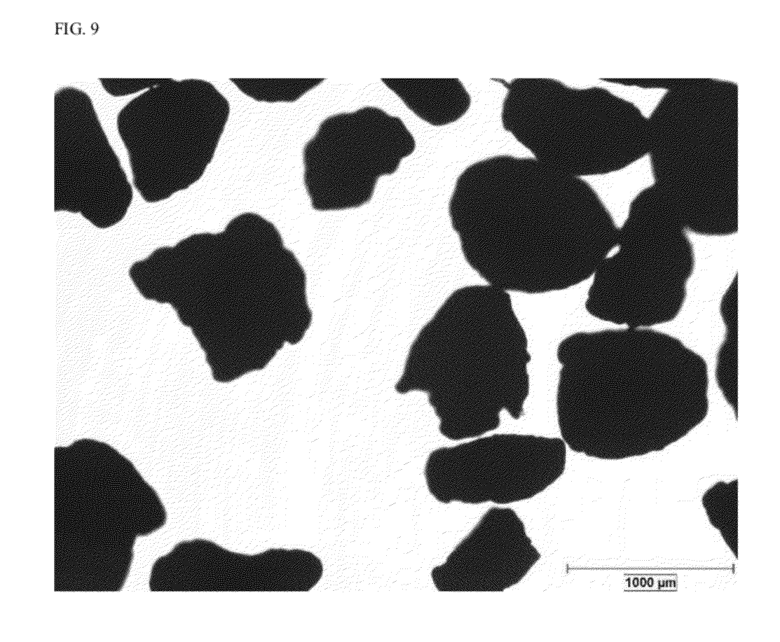

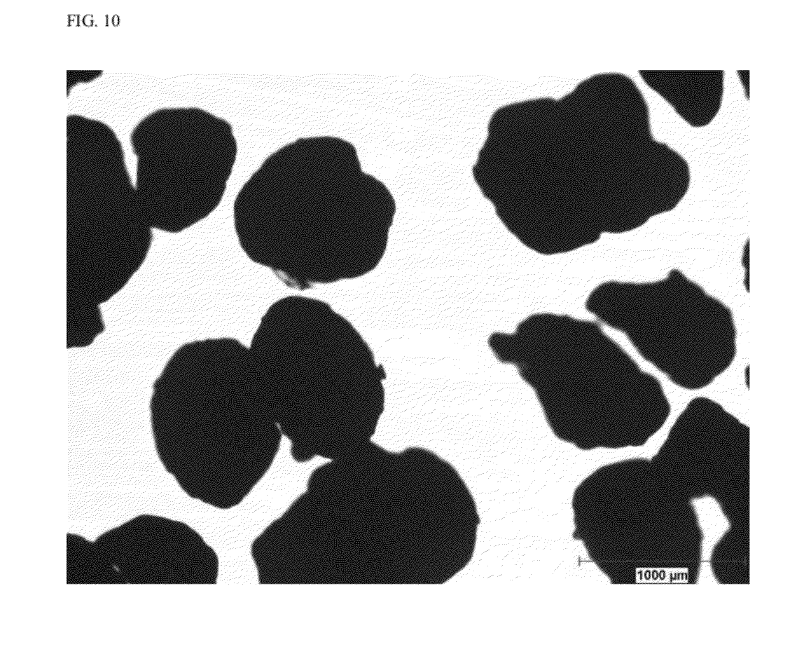

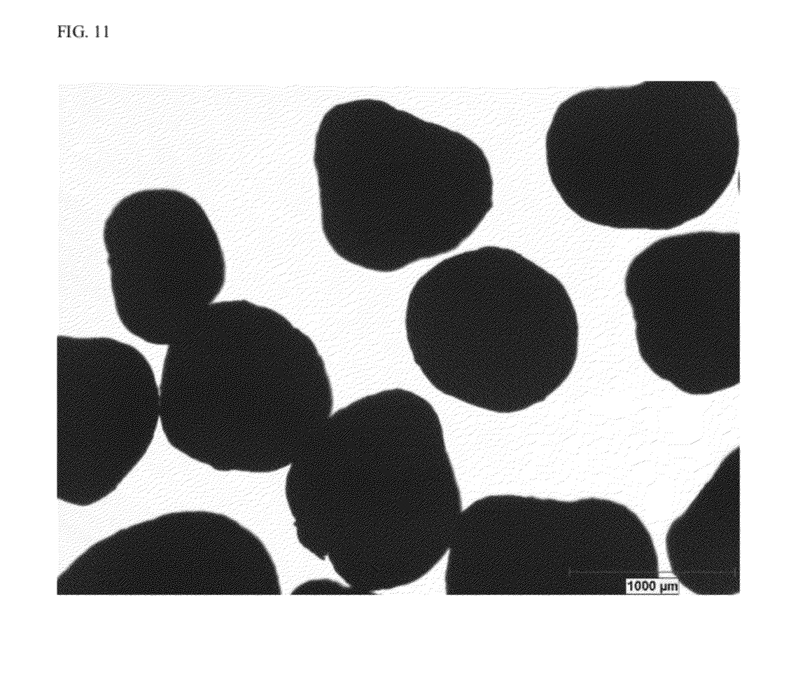

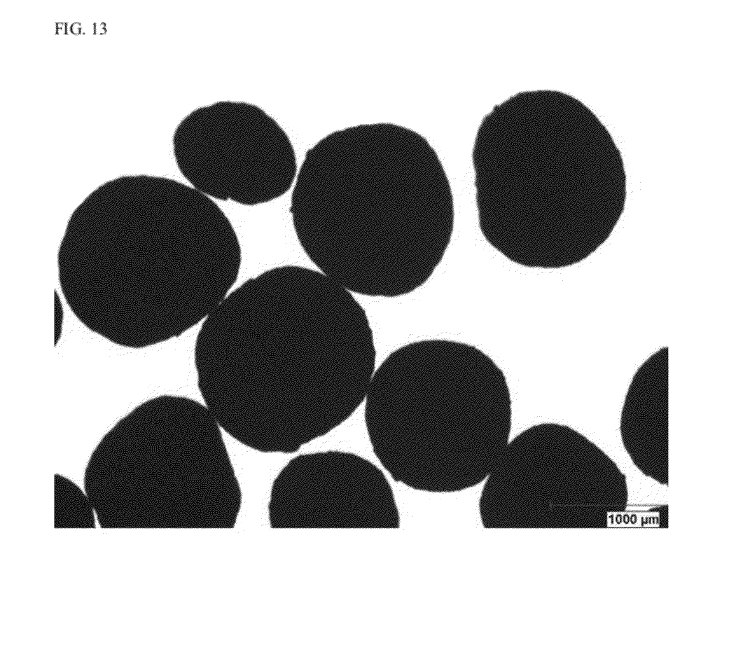

Formulations containing higher amounts of VIA and combinations of Carbopol and sodium alginate were evaluated. The use of granulation liquids other than water and ethanol led to friable and soft pellets non suitable for coating processes. Also, higher Carbopol load and Carbopol-sodium alginate combination formulations did not give well formed pellets. A complete impeding of the filtration using water and vodka as solvents could not be obtained with these new formulations. 25× magnification images of selected pellet formulations (pellets 0.5-1.0 mm) are shown in FIGS. 6-12. This fraction represented between 48% and 64% yields of the total pellets produced. The pellets from lot L066-01004 (Carbopol 971P), granulated with an aqueous solution containing CaCl₂ (FIG. 13) were used as a reference in terms of the pellet's shape.

### Example 5

### Evaluation of Carbopol 971P and Alkalining Agent Formulations

Formulations were prepared for determining the effects of alkalining agents. To prepare Carbopol formulations without alkalining agents at 100 to 200 g/batch, the powdered materials were first blended for about 1 minute and the mixture was sieved using a 20 mesh sieve. Powders premixing was completed in a Hobart Model N-50 planetary mixer for about 2 minutes at low speed (60 rpm) and about 45 seconds at 124 rpm. The granulating liquid (water or CaCl₂ aqueous solution) was slowly added into the mixture until all the material was granulated. The wet mass was then extruded immediately by dome extrusion using a LCI Multi Granulator MG-55 fitted with a 1.0 or 1.2 mm die and extrusion speed of 30 or 50 rpm. The extrudates were spheronized at speeds between 960 and 1800 rpm for up to 20 minutes using a LCI Marumerizer QJ-230T equipped with 2.0 mm friction plate. Pellets were enterically coated using the same procedure described previously.

To prepare formulations that included meglumine or sodium bicarbonate as an alkalining agent, the same procedure described above for the Carbopol pelletization was used, except that 1.0 mm die and extrusion speed at 50 rpm was used. Also, spheronization speeds between 250 and 500 rpm for up to 10 minutes were used.

The formulation compositions evaluated are shown in Table 10. The parameters of operation for the most promising formulations can be found in Table 11.

TABLE 11
Extrusion/Spheronization Parameters for Lot L066-01004 (Carbopol/CaCl₂),
-01022 (Meglumine), and -01023 (Carbopol).

| | Formulation | | | | |
|---|---|---|---|---|---|
| | | | | -01022A and | |
| Parameters | -01004A | -01004E | -01004G | -01022B | -01023 |

Dry Blending

| | | | | | |
|---|---|---|---|---|---|
| Batch size (g) | 100 | 100 | 100 | 100 | 200 |
| Pre-mix time (min) | 2 | 2 | 2 | 2 | 2 |
| Mixer speed (rpm) | 60 | 60 | 60 | 60 | 60 |

Wet Granulation

| | | | | | |
|---|---|---|---|---|---|
| Dose time (min:sec) | 0:53 | 0:57 | 1:18 | 1:17 | 4:18 |
| Total mix time | 1:38 | 2:02 | 2:18 | 2:17 | 6:10 |
| (min:sec) | | | | | |
| Mixer speed (rpm) | 60/124 (45 sec) | 60/124 (45 sec) | 60/124 (43 sec) | 60 | 60 |
| Liquid rate (g/min) | 45.3 | 43.0 | 30.9 | 43.2 | 20.9 |
| Granulation liquid (% | 40.0 | 40.0 | 40.0 | 55.5 | 45.0 |
| w/w) on dry basis | | | | | |

Extrusion (Dome)

| | | | | | |
|---|---|---|---|---|---|
| Shaft speed (rpm) | 50 | 50 | 30 | 50 | 30 |
| Die diameter (mm) | 1.0 | 1.0 | 1.0 | 1.0 | 1.0 |

Spheronization

| | | | | | |
|---|---|---|---|---|---|
| Plate speed (rpm) | 1800/1000 | 1800/1500 | 1800 | 500/250 | 960-1750 |
| Spheronization time | 9/9 | 8/10 | 20 | 3/3 | 17 |
| (min) | | | | | |

For the filtration/extraction testing, two extraction methods were used: dry grinding, and wet grinding. In the dry grinding method, Carbopol and meglumine pellets were grinded separately for 1 minute using mortar and pestle. 10 mL of solvent was added to different ratios of Carbopol/meglumine grinded pellets and the mixture was vigorously shaken for less than one minute and immediately filtrated through a coffee filter. After 10 minutes, the filtrate was weighed.

In the wet grinding method, different ratios of Carbopol/meglumine pellets were introduced in a mortar. The pellets were too hard to be easily crushed by hand. The solvent was then introduced into the mortar and the material was wet-milled in solvents. The mix was filtered through a coffee filter. After 10 minutes, the filtrate was weighed.

Carbopol 971P (13.5%) pellets, as shown in FIG. 13, could be produced using a CaCl₂ aqueous solution as granulating liquid. CaCl₂ reduced in-process viscosity of the Carbopol and allowed proper yield. However, CaCl₂ also reduced the swelling properties of Carbopol during extraction testing. This could be prevented by adding an alkalining agent such as meglumine within the formulation. In formulation lot L066-01023, Carbopol 971P percentage was decreased from 13.5% to 10% and pellets were produced with pure water as granulating liquid avoiding the use of CaCl₂ (see Table 10). Meglumine-based pellets were produced (lot L066-01022) separately in order to avoid in process swelling of Carbopol. Table 12 shows that the use of a 60:40 ratio of Carbopol and meglumine pellets from lots L066-01004 and L066-01022, as well as a 70:30 ratio of Carbopol and meglumine pellets from lots L066-01023 and L066-01024, led to viscous aqueous solutions and reduced filtration rate.

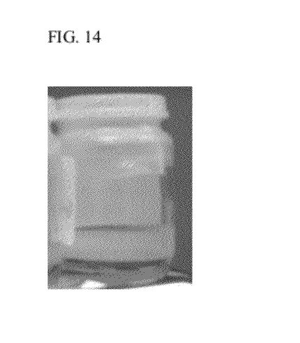

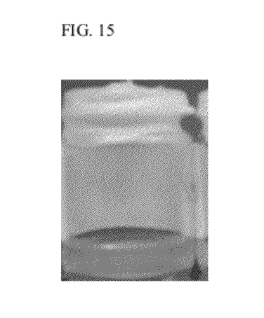

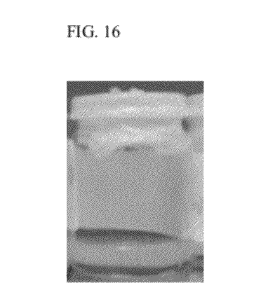

Extraction results depended mainly on the extraction approach. Using a coffee grinder for 1 min, mixing with solvent by shaking for a few seconds and immediate filtrating through a coffee filter, the mixtures could be filtered as pellets integrity was maintained. In these tests it was found that the filtrates consisted of a cloudy liquid (FIGS. 14, 15 and 16) containing all caffeine. Although, by pulverisation using mortar and pestle, filtration was not possible using water and vodka. Sodium bicarbonate was also evaluated as an alkalining agent. Pellets containing 80% of sodium bicarbonate (L066-01024) did not show the same behavior as compared to meglumine (17-20%) pellets in enhancing the swelling of Carbopol.

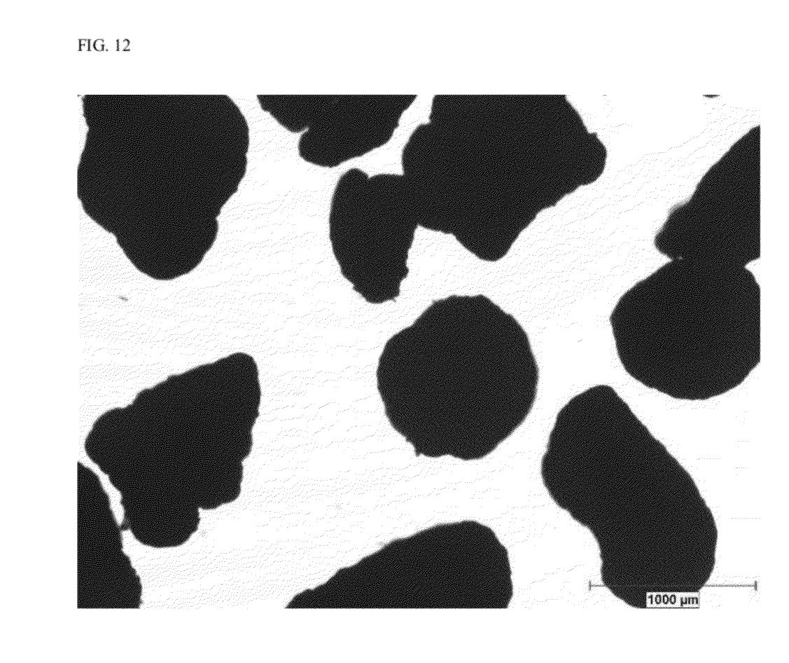

TABLE 12
Carbopol and Meglumine Formulations* Filtration/Extraction Results.

| | | | | | | Caffeine in |
|---|---|---|---|---|---|---|
| Lot | CPL/AA | | | Method of | Filtrate wt | the filtrate |
| L066 | Pellets (%) | Solid phase (g) | Solvent | tampering | after 10 min | (mg/mL) |
| -01004A | CPL_{UP}: 13.5 | 004A: 0.5 (T = 0) | Water: | (a) Grinding^{CG}; | Water: | NA |
| | | | 10 mL | (b) Dissolving; and | Filterable | |
| | | | | (c) Filtration with | | |
| | | | | coffee filter | | |
| | | 004A: 0.5 | Water: | (a) Crushing^{MP} with | Water: 1.6 g | NA |
| | | (2 months) | 10 mL | solvent; and | | |
| | | | | (b) Filtration with | | |
| | | | | coffee filter | | |
| | | 004A: 0.5 | Water: | (a) Crushing^{MP} with | Water: 0.0 g | NA |
| | | (2 months) + | 10 mL | solvent; and | Vodka: 0.0 g | |
| | | AA: 0.05 | Vodka: | (b) Filtration with | | |
| | | | 10 mL | coffee filter | | |
| | | 004A: 0.5 | Water: | (a) Crushing^{MP} with | Water: 1.2 g | NA |
| | | (2 months) + | 10 mL | solvent; and | | |
| | | AA: 0.05) | | (b) Filtration with | | |
| | | | | gauze bandage | | |
| -01004GOA- | CPL_{CP}: 11.1** | 004GOA: 0.3 | Water: | (a) Grinding^{CG}; | Water: 1.4 g | NA |
| -01022AOA | Meg_{CP}: 17.4*** | 022AOA: 0.2 | 10 mL | (b) Dissolving; and | | |
| | | (T = 0) | | (c) Filtration with | | |
| | | | | coffee filter | | |
| | | 004GOA: 0.6 | Water: | (a) Grinding^{CG}; | Water: 1.4 g | NA |
| | | 022AOA: 0.4 | 10 mL | (b) Dissolving; and | | |
| | | (T = 0) | | (c) Filtration with | | |
| | | | | coffee filter | | |
| | | 004GOA: 0.3 | Water: | (a) Crushing^{MP} with | Water: 0.2 g | NA |
| | | 022AOA: 0.2 | 10 mL | solvent; and | Vodka: 0.3 g | |
| | | (T = 0) | Vodka: | (b) Filtration with | | |
| | | | 10 mL | coffee filter | | |
| | | 004GOA: 0.6 | Water: | (a) Crushing^{MP} with | Water: 0.0 g | NA |
| | | 022AOA: 0.4 | 10 mL | solvent; and | Vodka: 0.0 g | |
| | | (T = 0) | Vodka: | (b) Filtration with | | |
| | | | 10 mL | coffee filter | | |
| -01004GOA- | CPL_{CP}: 11.1 | 004GOA: 0.3 | Water: | (a) Grinding^{CG}; | Water: 4.1 g | Water: |
| -01022AOA | Meg_{CP}: 17.4 | 022AOA: 0.2 | 10 mL | (b) Dissolving; and | Vodka: 4.0 g | 8.4(FIG. 12) |
| | | MCC-Caf: 0.5 | Vodka: | (c) Filtration with | | Vodka: 9.7 |
| | | (T = 0) | 10 mL | coffee filter | | (FIG. 13) |
| | | 004GOA: 0.6 | Water: | (a) Grinding^{CG}; | Water: 2.1 g | Water: 9.5 |
| | | 022AOA: 0.4 | 10 mL | (b) Dissolving; and | | (FIG. 14) |
| | | MCC-Caf: 0.5 | | (c) Filtration with | | |
| | | (T = 0) | | coffee filter | | |
| | | 004GOA: 0.3 | Water: | (a) Crushing^{MP} with | Water: 0.3 g | NA |
| | | 022AOA: 0.2 | 10 mL | solvent; and | | |
| | | MCC-Caf: 0.5 | | (b) Filtration with | | |
| | | (T = 0) | | coffee filter | | |
| -01004E | CPL_{UP}: 13.5 | 004E: 0.5 AA: | Water: | (a) Grinding^{CG}; | Water: 1.6 g | NA |
| | | 0.05 (T = 0) | 10 mL | (b) Dissolving; and | | |
| | | | | (c) Filtration with | | |
| | | | | coffee filter | | |
| -01023- | CPL_{UP}: 10.0 | 023: 0.7 | Water: | (a) Crushing^{MP} with | Water: 0.0 g | NA |
| -01022B | Meg_{UP}: 20.0 | 022B: 0.3 | 10 mL | solvent; and | | |
| | | | | (b) Filtration with | | |
| | | | | coffee filter | | |
| -01023- | CPL_{UP}: 10.0 | 23: 0.7 | Water: | (a) Crushing^{MP} with | Water: 2.5 g | NA |
| -01024 | NaHCO_{3 UP}: 80.0 | 24: 0.3 | 10 mL | solvent; and | | |
| | | | | (b) Filtration with | | |
| | | | | coffee filter | | |

*Carbopol pellets (lot L0066-01004GOA) and meglumine pellets (lot L0066-01022AOA) were used.
UP: uncoated pellets;
CP: coated pellets;
AA: raw meglumine as alkalining agent;
CG: Coffee grinder;
MP: Mortar/pestle,
Filterable: all liquid pass through the filter;
NA: not applicable, filtrate ≦1.0 g or no caffeine in the mix.
**11.1% = 13.0% pellets/(100 + 4% (Opadry coating) + 13% (Acryl-Eze coating)).
***17.4% = 20.0% pellets/(100 + 3% (Opadry coating) + 12% (Acryl-Eze coating)).

### Example 6

### Stability Testing

Enteric coated-pellets formulations were placed under accelerated and long term stability programs in closed HDPE containers. Stability was tested for pellets containing xanthan gum, Carbopol, and sodium alginate. Throughout the study, the filtration rate was evaluated by collecting filtrates for 10 minutes through a coffee filter. The solid phase consisted of 0.5 g of a mixture of caffeine-MCC and 0.5 g of powdered pellets, which was dispersed in 10 mL of extraction liquid. Grinding of the pellets was accomplished with a mortar and pestle and caffeine extractions were performed using water and vodka as extraction liquids.

Stability results are shown in Tables 13, 14, 15. In general, the results showed that the filtration rate depended on the degree of grinding. Differences were observed in the filtration rate for the pellets grinded using a coffee grinder and crushed using a mortar and pestle. Due to the pellets size and enteric coating thickness, pellets cannot be pulverized properly using a coffee grinder, unless a large quantity is used. It can be assumed that the same phenomenon will be observed with pellets containing opioids. Use of a dry mortar and pestle led to even more time consuming and difficult pulverization of the pellets. Use of wet mortar and pestle led to easier pulverization although the filtration rates for xanthan gum-coated pellets (e.g., lot L066-01008PC, Table 13) and Carbopol-coated pellets (e.g., lot L066-010113PC, Table 14) dropped to near, or close to 0 mL/min, for many samples.

After 4 weeks at 40° C./75% RH, the xanthan gum-coated pellets (e.g., lot L066-01008PC, Table 13) showed results comparable to those observed for non-exposed samples. However, the proprieties of Carbopol-coated pellets (e.g., lot L066-01013PC, Table 14) were slightly affected by the storage time. For the pellets grinded using a coffee grinder and stored under laboratory conditions, the filtration rate was 0, 0.2 and 0.9 mL/10 minutes at T=0, 4 and 6 weeks, respectively (e.g., Table 14, Samples 130WG, 131WG, and 136wWG, respectively). Grinding method efficiency can be appreciated with this lot with filtration rates of 2.2 and 0.2 mL/10 minutes after 1 month under laboratory conditions for the pellets crushed using mortar and coffee grinder, respectively (e.g., Table 14, Samples 131WM and 131WG, respectively). The loss of properties of this formulation could be due to incomplete pulverization of the pellets using dry mortar and pestle.

Using the wet mortar and pestle grinding method, xanthan gum (XG)-based formulations (e.g., Table 13, lot L066-01008PC) produced very viscous suspensions and impeded caffeine extraction from water and vodka up to 4 months under accelerated (40° C./75% RH) and long term (25° C./60% RH) conditions (e.g., Samples 0844075WM, 0844075VM, 0842560WM, and 0842560VM).

TABLE 13
Extraction of Caffeine (10 mg/mL) from Lot L066-01008PC Stability Samples.

| | VIA | | Extracting Solvent/ | Filtration rate | Caffeine in the |
|---|---|---|---|---|---|
| Sample | (mg/mL) | Storage | Grinding | (mL/10 min) | filtrate (mg/mL) |
| 080WG | XG | T = 0 | Water/Coffee grinder | 0.0 | NA |
| 080VG | 9.0 | T = 0 | Vodka/Coffee grinder | 2.4 | 8.3 |
| 081WG | | T = 1 month Lab. | Water/Coffee grinder | 0.0 | NA |
| | | conditions | | | |
| 081WM | | T = 1 month Lab. | Water/Mortar (dry) | 0.5 | Not tested |
| | | conditions | | | |
| 0814075WM | | T = 1 month | Water/Mortar (dry) | 0.4 | Not tested |
| | | 40° C./75% RH | | | |
| 0814075VM | | T = 1 month | Vodka/Mortar (dry) | 2.0 | 12.5 |
| | | 40° C./75% RH | | | |
| 0824075WM | | T = 2 months | Water/Mortar (wet) | 0.0 | NA |
| | | 40° C./75% RH | | | |
| 0824075VM | | T = 2 months | Vodka/Mortar (wet) | 0.0 | NA |
| | | 40° C./75% RH | | | |
| 0834075WM | | T = 3 months | Water/Mortar (wet) | 0.0 | NA |
| | | 40° C./75% RH | | | |
| 0834075VM | | T = 3 months | Vodka/Mortar (wet) | 0.0 | NA |
| | | 40° C./75% RH | | | |
| 0832560WM | | T = 3 months | Water/Mortar (wet) | 0.0 | NA |
| | | 25° C./60% RH | | | |
| 0832560VM | | T = 3 months | Vodka/Mortar (wet) | 0.0 | NA |
| | | 25° C./60% RH | | | |
| 0844075WM | | T = 4 months | Water/Mortar (wet) | 0.0 | NA |
| | | 40° C./75% RH | | | |
| 0844075VM | | T = 4 months | Vodka/Mortar (wet) | 0.0 | NA |
| | | 40° C./75% RH | | | |
| 0842560WM | | T = 4 months | Water/Mortar (wet) | 0.0 | NA |
| | | 25° C./60% RH | | | |
| 0842560VM | | T = 4 months | Vodka/Mortar (wet) | 0.0 | NA |
| | | 25° C./60% RH | | | |

Not tested: filtrates were tested by HPLC only when 1 gram or more of filtrate was recovered after 10 minutes.
NA: not applicable, no fluid passed through the coffee filter.
Solvent/Mortar (dry): Pellets crushed using mortar and pestle, powdered material (0.5 g) is introduced in a bottle and 0.5 g of a Caffeine (20% w/w)-MCC-101 is added. The solvent (10 mL) is added and the bottle is vigorously shaken for a few seconds.
Solvent/Mortar (wet): 0.5 grams of pellets are weighed and introduce in a mortar, 0.5 grams of a Caffeine (20% w/w)-MCC-101 is added. The solvent (10 mL) is added. The mixture is vigorously crushed with the pestle until the pellets are well crushed.
Filtration rate: The solid is separated from the liquid phase by passing the mixture over a filter (coffee filter). After 10 minutes the liquid passing through the filter (filtrate) is weighed.

Carbopol 917P (CPL) based pellets (e.g., Table 14) produced slightly less viscous suspensions than xanthan gum pellets (e.g., Table 13) but in general blocked filtration. After 3 months of storage, a filtration rate of between 0 and 0.4 mL/10 minutes was observed for various samples. But after 4 months of storage under accelerated conditions, 1 mL of a cloudy liquid filtrate was recovered after 10 minutes using water as the extraction liquid (e.g., Table 14, Sample 1344075WM). This 1 ml of filtrate contained a large quantity of caffeine (9 mg).

TABLE 14
Extraction of Caffeine (10 mg/mL) for Lot L066-01013PC Stability Samples.

| | VIA | | Extraction Solvent/ | Filtration rate | Caffeine in the |
|---|---|---|---|---|---|
| Sample | (mg/mL) | Storage | Grinding | (mL/10 min) | filtrate (mg/mL) |
| 130WG | CPL | T = 0 | Water/Coffee grinder | 0.0 | NA |
| 130VG | 5.5 | T = 0 | Vodka/Coffee grinder | 0.3 | Not tested |
| 131WG | | T = 1 month Lab. | Water/Coffee grinder | 0.2 | Not tested |
| | | conditions | | | |
| 131WM | | T = 1 month Lab. | Water/Mortar (dry) | 2.2 | 9.0 |
| | | conditions | | | |
| 1314075W | | T = 1 month | Water/Mortar (dry) | 2.5 | 8.9 |
| | | 40° C./75% RH | | | |
| 1314075V | | T = 1 month | Vodka/Mortar (dry) | 1.9 | 12.2 |
| | | 40° C./75% RH | | | |
| 136wWG | | T = 6 weeks Lab. | Water/Coffee grinder | 0.9 | Not tested |
| | | conditions | | | |
| 1324075W | | T = 2 months | Water/Mortar (wet) | 0.0 | NA |
| | | 40° C./75% RH | | | |
| 1324075V | | T = 2 months | Vodka/Mortar (wet) | 0.0 | NA |
| | | 40° C./75% RH | | | |
| 1334075WM | | T = 3 months | Water/Mortar (wet) | 0.4 | Not tested |
| | | 40° C./75% RH | | | |
| 1334075VM | | T = 3 months | Vodka/Mortar (wet) | 0.1 | Not tested |
| | | 40° C./75% RH | | | |
| 1332560WM | | T = 3 months | Water/Mortar (wet) | 0.1 | Not tested |
| | | 25° C./60% RH | | | |
| 1332560VM | | T = 3 months | Vodka/Mortar (wet) | 0.0 | NA |
| | | 25° C./60% RH | | | |
| 1344075WM | | T = 4 months | Water/Mortar (wet) | 1.0 | 9.0 |
| | | 40° C./75% RH | | | |
| 1344075VM | | T = 4 months | Vodka/Mortar (wet) | 0.0 | NA |
| | | 40° C./75% RH | | | |
| 1342560WM | | T = 4 months | Water/Mortar (wet) | 0.0 | NA |
| | | 25° C./60% RH | | | |
| 1342560VM | | T = 4 months | Vodka/Mortar (wet) | 0.0 | NA |
| | | 25° C./60% RH | | | |

Not tested: filtrates were tested by HPLC only when 1 gram or more of filtrate was recovered after 10 minutes.
NA: not applicable, no fluid passed through the coffee filter.
Solvent/Mortar (dry): Pellets crushed using mortar and pestle, powdered material (0.5 g) is introduced in a bottle and 0.5 g of a Caffeine (20% w/w)-MCC-101 is added. The solvent (10 mL) is added and the bottle is vigorously shaken for a few seconds.
Solvent/Mortar (wet): 0.5 grams of pellets are weighed and introduce in a mortar, 0.5 grams of a Caffeine (20% w/w)-MCC-101 is added. The solvent (10 mL) is added. The mixture is vigorously crushed with the pestle until the pellets are well crushed.
Filtration rate: The solid is separated from the liquid phase by passing the mixture over a filter (coffee filter). After 10 minutes the liquid passing through the filter (filtrate) is weighed.

Table 15 shows the results for sodium alginate (SA) based pellet formulations. The results after 4 weeks of storage at 40° C. and 75% RH were better in terms of impeding caffeine extraction compared to initial results and stabilized at future timepoints. Samples stored at 25° C. and 60% RH for 3 months showed deterrent effects with water but not with vodka. For all samples, the filtrates consisted of a cloudy suspension containing caffeine. The tests performed after 4 months showed that sodium alginate based pellets reduced the filtration rate from 10 to 1.6-4 mL but the solutions contained large amounts of caffeine.

TABLE 15
Extraction of Caffeine (10 mg/mL) for Lot L066-01015-18PC Stability.

| | VIA | | Extraction Solvent/ | Filtration rate | Caffeine in the |
|---|---|---|---|---|---|
| Sample | (mg/mL) | Storage | Grinding | (mL/10 min) | filtrate (mg/mL) |
| 15180W | SA | T = 0 | Water/Coffee grinder | 1.7 | 0.0 |
| 15180V | 18.0 | T = 0 | Vodka/Coffee grinder | 5.3 | 9.7 |
| 151814075W | | T = 1 month | Water/Mortar (dry) | 0.8 | Not tested |
| | | 40° C./75% RH | | | |
| 151814075V | | T = 1 month | Vodka/Mortar (dry) | 2.9 | 11.2 |
| | | 40° C./75% RH | | | |
| 151824075W | | T = 2 months | Water/Mortar (wet) | 1.4 | 9.3 |
| | | 40° C./75% RH | | | |
| 151824075V | | T = 2 months | Vodka/Mortar (wet) | 2.5 | 10.5 |
| | | 40° C./75% RH | | | |
| 151834075WM | | T = 3 months | Water/Mortar (wet) | 1.3 | 7.6 |
| | | 40° C./75% RH | | | |
| 151834075VM | | T = 3 months | Vodka/Mortar (wet) | 3.2 | 9.7 |
| | | 40° C./75% RH | | | |
| 151832560WM | | T = 3 months | Water/Mortar (wet) | 0.7 | Not tested |
| | | 25° C./60% RH | | | |
| 151832560VM | | T = 3 months | Vodka/Mortar (wet) | 4.1 | 8.7 |
| | | 25° C./60% RH | | | |
| 151834075WM | | T = 4 months | Water/Mortar (wet) | 1.6 | 9.66 |
| | | 40° C./75% RH | | | |
| 151834075VM | | T = 4 months | Vodka/Mortar (wet) | 4.0 | 8.14 |
| | | 40° C./75% RH | | | |
| 151832560WM | | T = 4 months | Water/Mortar (wet) | 2.0 | 8.46 |
| | | 25° C./60% RH | | | |
| 151832560VM | | T = 4 months | Vodka/Mortar (wet) | 3.4 | 8.98 |
| | | 25° C./60% RH | | | |

Not tested: filtrates were tested by HPLC only when 1 gram or more of filtrate was recovered after 10 minutes.
NA: not applicable, no fluid passed through the coffee filter.
Solvent/Mortar (dry): Pellets crushed using mortar and pestle, powdered material (0.5 g) is introduced in a bottle and 0.5 g of a Caffeine (20% w/w)-MCC-101 is added. The solvent (10 mL) is added and the bottle is vigorously shaken for a few seconds.
Solvent/Mortar (wet): 0.5 grams of pellets are weighed and introduce in a mortar, 0.5 grams of a Caffeine (20% w/w)-MCC-101 is added. The solvent (10 mL) is added. The mixture is vigorously crushed with the pestle until the pellets are well crushed.

Filtration rate: The solid is separated from the liquid phase by passing the mixture over a filter (coffee filter). After 10 minutes the liquid passing through the filter (filtrate) is weighed.

Pellet stability was also tested in pellets containing Carbopol and meglumine. The filtration rate was evaluated by collecting filtrates for 10 minutes through a coffee filter. The solid phase consisted of 0.5 g of a mixture of caffeine (20%)-MCC, 0.3 grams of Carbopol coated pellets and 0.2 grams of meglumine coated pellets. The extraction was carried out in 10 mL of extraction solvent (water or vodka) by grinding with a mortar and pestle until the pellets were completely crushed. The results are provided in Table 16.

TABLE 16
Carbopol (11.1% w/w) lot: L066-01004GOA and Meglumine (17.4% w/w) lot:
L066-01022AOA Stability Results.

| | | | | Filtrate | Caffeine | Caffeine | |
|---|---|---|---|---|---|---|---|
| | Solvent | | IVA/AA* | (g) after | Assay | Recovery | |
| Conditions | (10 mL) | Solid phase (g) | (mg/mL) | 10 min | (mg/mL) | (%) | Observations |
| T = 0 | Water | Carbopol pellets | CPL 3.3 | 0.3 | Not tested | — | — |
| 1 month | Water | (lot -004GOA): | AA 3.5 | 0.7 | Not tested | — | Meglumine |
| 40° C./75% RH | Vodka | 0.3 | | 3.1 | 8.9 | 28 | pellets color |
| | | Meglumine | | | | | changed from |
| | | pellets: (lot - | | | | | off white to |
| | | 022AOA): 0.2 | | | | | yellowish |
| 1 month | Water | MCC-Caffeine | | 0.0 | NA | — | No change of |
| 25° C./60% RH | Vodka | (20%): 0.5 | | 0.5 | Not tested | — | pellets color |
| | | | | | | | was observed |
| 2 months | Water | | | 2.9 | 10.0 | 29 | Meglumine |
| 40° C./75% RH | Vodka | | | 4.1 | 9.5 | 39 | pellets color |
| | | | | | | | yellowish |
| 3 months | Water | | | 5.0 | 9.6 | 48 | Meglumine |
| 40° C./75% RH | Vodka | | | 4.3 | 9.1 | 39 | pellets color |
| | | | | | | | yellowish |
| 3 months | Water | | | 2.4 | 9.0 | 22 | No change of |
| 25° C./60% RH | Vodka | | | 0.9 | Not tested | — | pellets color |
| | | | | | | | was observed |

*Carbopol pellets (CPL) as IVA = 0.3 grams × (0.111) × 1000/10 mL = 3.3 mg/mL; Meglumine pellets as AA = 0.2 grams × (0.174) × 1000/10 mL = 3.5 mg/mL.

Carbopol/meglumine pellets stored at 40° C. and 75% RH for 1 month and more were unable to impede completely the filtration and extraction of caffeine. For these pellets the humidity/temperature conditions during storage affected remarkably their effectiveness. The meglumine pellets showed color changes which could be a sign of degradation. As for previous extraction testing, the filtrates resulted in a cloudy suspension (as samples showed in FIGS. 14-16). The efficacy of Carbopol/meglumine pellets system to prevent the filtration and extraction of caffeine remained after 1 month under 25° C./60% RH. However, this behaviour decreased with storage time. After 3 months at 25° C. and 60% RH about 22% of caffeine was recovered within 10 minutes. This could probably be due to the exposure to humidity above 60%. Indeed, samples from same lots, keep for 3 months under laboratory temperature/humidly conditions (22.5±0.1° C./35±2% RH) were used for testing shown in Tables 17 and 18 and showed less than 10% of caffeine recovery (e.g., Samples 1-1 and 1-2 in Table 17 and V1-1 and V1-2 in Table 18). The use of a desiccant for long term storage is a viable solution and should be considered.

### Example 7

### Impact of Characteristics of the Pellets and/or Extraction Method

This study investigated how the amount of pellets (0.5 or 1.0 g), Carbopol/meglumine pellets ratio (0.3/0.2 and 0.6/0.4, or 0.7/0.3), volume of the solvent (10, 20, or 50 mL), and filtering media (coffee filter paper or cotton balls) can impact caffeine extraction when (a) water is used as the extraction liquid, or (b) vodka is used as the extraction liquid. The results are shown in Tables 17 and 18.

The extraction method used in Tables 17 and 18 involved mixing the dry ingredients (pellets and MCC-caffeine mix) and water using a mortar and pestle until the pellets were crushed. For these experiments, Carbopol pellets (lot L0066-01004GOA) and meglumine pellets (lot L0066-01022AOA) were used.

(a) Water as the Extraction Liquid

A mixture of Carbopol/meglumine pellets in amounts of 0.5 g and 1.0 g were added to 0.5 g of MCC-caffeine mix (containing 100 mg caffeine). Using a Carbopol/meglumine ratio of 0.3/0.2 or 0.6/0.4, between 9 and 11% of caffeine was extracted for 0.5 g pellets mixtures (Samples 1-1 and 1-2), while only 2% of the drug was extracted for 1.0 g pellet mixtures (Samples 4-1 and 4-2). The caffeine component did not affect the filtration rate, as confirmed by the poor extraction results observed with tests 2-5 and 2-6 (non-caffeine containing mixtures).

No significant differences were found using a Carbopol/meglumine ratio of 0.3/0.2 or 0.6/0.4 (Samples 1 and 4), versus a ratio of 0.7/0.3 (Sample 2) using 10 mL of water.

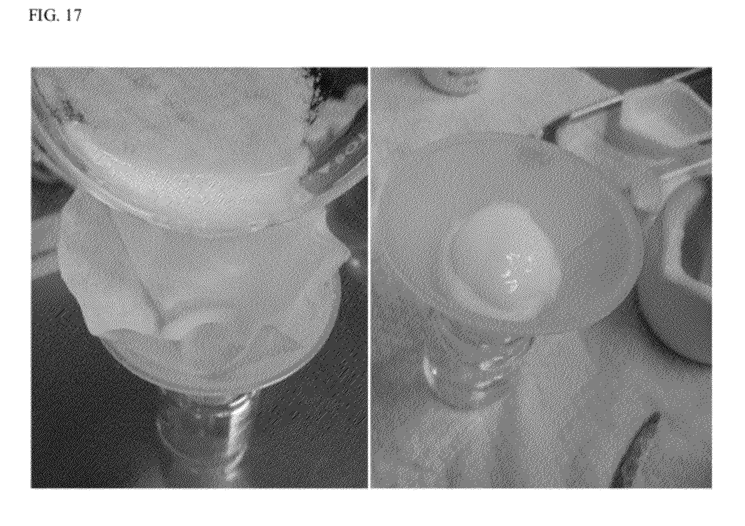

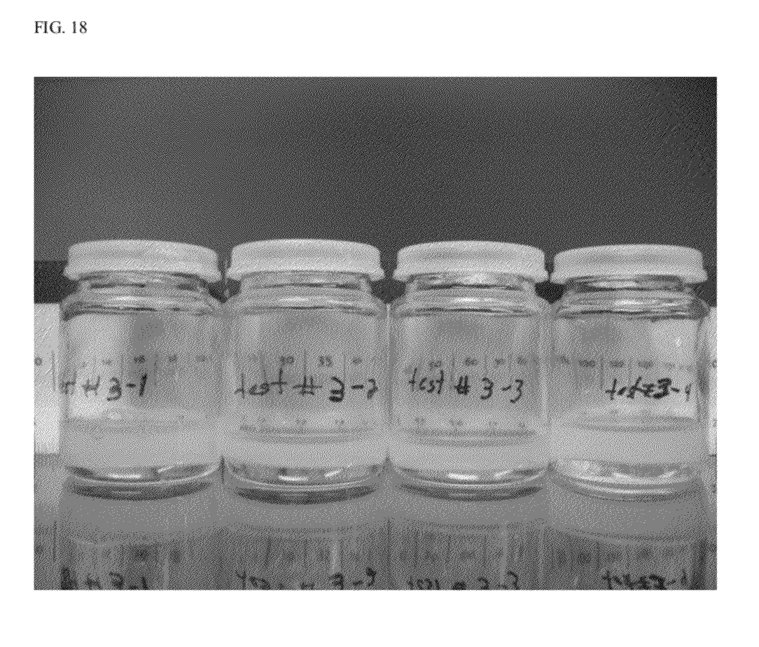

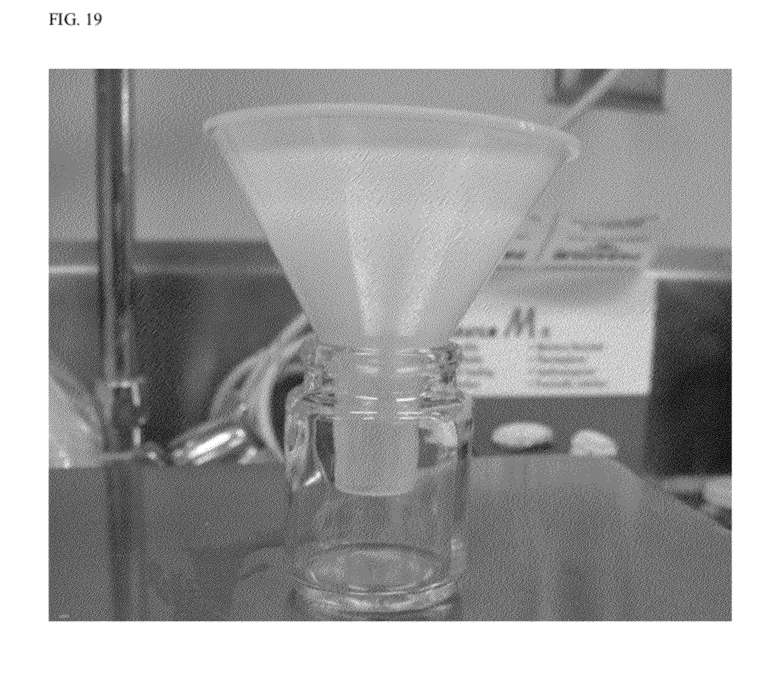

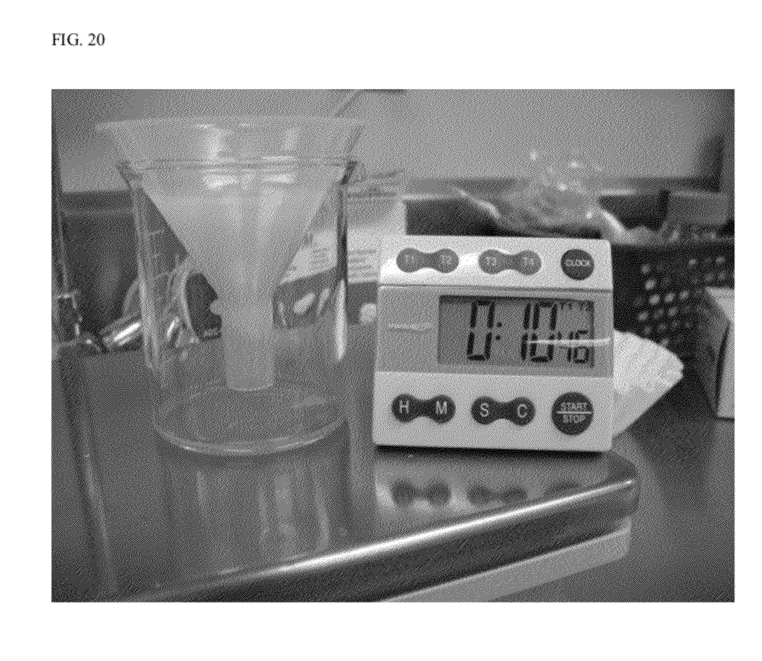

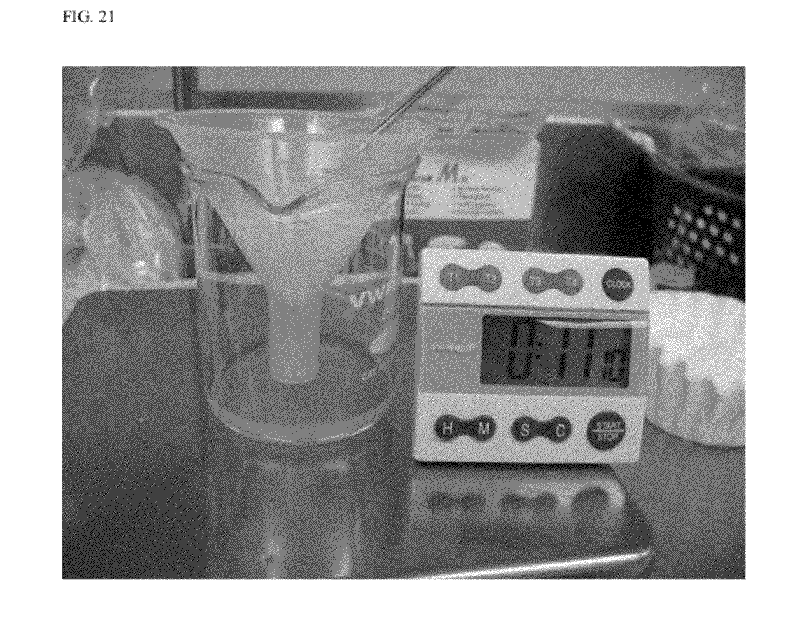

The volume of solvent, i.e., water, may influence caffeine extraction. After crushing with 10 mL of water, very viscous suspensions were obtained by mixing 0.5 g of a MCC-caffeine mixture containing 100 mg of caffeine with 0.5 and 1.0 g of Carbopol/meglumine pellets mix (FIG. 17). When 10 mL of water was used as the solvent, between 0.0 and 1.1 g of filtrate containing about 10 mg/mL can be recovered, which represents a recovery of 0 to 11% of total available caffeine in the mixture. When 20 mL of water was used, between 15 and 26% of caffeine could be extracted. For 50 mL of water, up to 31% of caffeine was extracted, depending on the filtering medium. However, independently of the filtration system, all testing containing Carbopol/meglumine pellets resulted in cloudy filtrates (FIGS. 18-22).

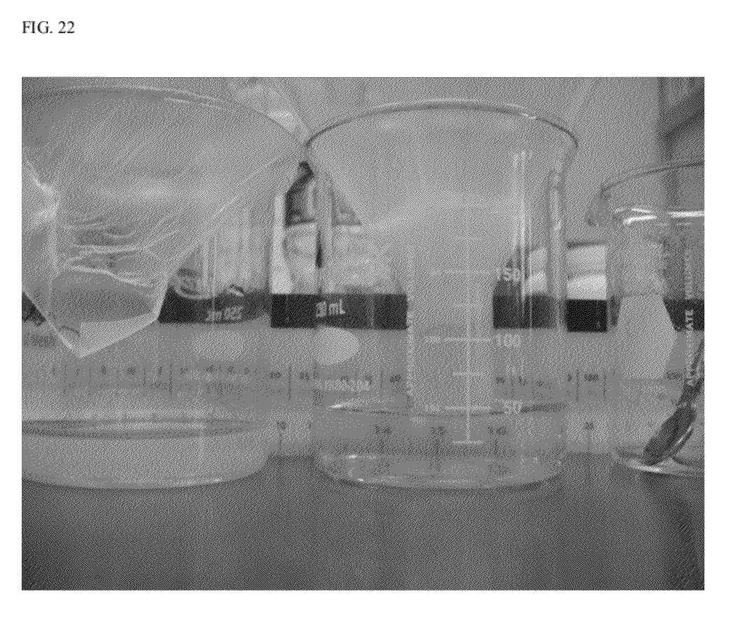

A clear and transparent caffeine aqueous solution (2 to 10 mg/mL) could not be obtained by filtering a caffeine/Carbopol/meglumine formulation with coffee filters or cotton balls, in one or several filtration steps (FIG. 22).

Caffeine, Carbopol and meglumine are soluble in water and thereby cannot be separated using the current extraction methods. The cloudy suspensions were stable and did not decant for 72 h. Moreover, heating the suspension led to a cloudy-white medium.

TABLE 17
Extraction Results for MCC-Caffeine with Carbopol* (11.1% w/w) and
Meglumine (17.4% w/w) System in Water.

| | | | | | | |
|---|---|---|---|---|---|---|
| | | | | Caffeine | | |
| | Solid Phase | Water | Filtrate (g) | Theoretical | Caffeine | |
| Sample | (g) | volume (mL) | after 10 min | (mg/mL) | Recovery (%) | Observations |

Using a Coffee Filter

| | | | | | | |
|---|---|---|---|---|---|---|
| 1-1 | MCC-Caf: 0.5 | 10 | 0.9 | 10.0 | 9.0 | Cloudy-white |
| | 004GOA: 0.3** | | | | | |
| | 022AOA: 0.2 | | | | | |
| 1-2 | MCC-Caf: 0.5 | 10 | 0.8 | 10.0 | 8.0 | Cloudy-white |
| | 004GOA: 0.3 | | | | | |
| | 022AOA: 0.2 | | | | | |
| 2-1 | MCC-Caf: 0.5 | 10 | 0.0 | NA | NA | NA |
| | 004GOA: 0.7** | | | | | |
| | 022AOA: 0.3 | | | | | |
| 2-2 | MCC-Caf: 0.5 | 10 | 0.0 | NA | NA | NA |
| | 004GOA: 0.7 | | | | | |
| | 022AOA: 0.3 | | | | | |
| 2-3 | MCC-Caf: 0.5 | 10 | 1.0 | 10.0 | 10.0 | Cloudy-white |
| | 004GOA: 0.7 | | | | | |
| | 022AOA: 0.3 | | | | | |
| 2-4 | MCC-Caf: 0.5 | 10 | 1.1 | 10.0 | 11.0 | Cloudy-white |
| | 004GOA: 0.7 | | | | | |
| | 022AOA: 0.3 | | | | | |
| 2-5 | 004GOA: 0.7 | 10 | 0.9 | NA | NA | Cloudy-white |
| | 022AOA: 0.3 | | | | | |
| 2-6 | 004GOA: 0.7 | 10 | 0.2 | NA | NA | Cloudy-white |
| | 022AOA: 0.3 | | | | | |
| 3-1 | MCC-Caf: 0.5 | 20 | 4.7 | 5.0 | 23.5 | Cloudy-white |
| | 004GOA: 0.7 | | | | | |
| | 022AOA: 0.3 | | | | | |
| 3-2 | MCC-Caf: 0.5 | 20 | 3.0 | 5.0 | 15.0 | Cloudy-white |
| | 004GOA: 0.7 | | | | | |
| | 022AOA: 0.3 | | | | | |
| 3-3 | MCC-Caf: 0.5 | 20 | 4.4 | 5.0 | 22.0 | Cloudy-white |
| | 004GOA: 0.7 | | | | | |
| | 022AOA: 0.3 | | | | | |
| 3-4 | MCC-Caf: 0.5 | 20 | 5.2 | 5.0 | 26.0 | Cloudy-white |
| | 004GOA: 0.7 | | | | | |
| | 022AOA: 0.3 | | | | | |
| 4-1 | MCC-Caf: 0.5 | 10 | 0.2 | 10.0 | 2.0 | Cloudy-white |
| | 004GOA: 0.6** | | | | | |
| | 022AOA: 0.4 | | | | | |
| 4-2 | MCC-Caf: 0.5 | 10 | 0.0 | NA | NA | NA |
| | 004GOA: 0.6 | | | | | |
| | 022AOA: 0.4 | | | | | |
| 5-1 | MCC-Caf: 0.5 | 20 | 2.8 | 5.0 | 14.0 | Cloudy-white |
| | 004GOA: 0.6 | | | | | |
| | 022AOA: 0.4 | | | | | |
| 5-2 | MCC-Caf: 0.5 | 20 | 2.4 | 5.0 | 12.0 | Cloudy-white |
| | 004GOA: 0.6 | | | | | |
| | 022AOA: 0.4 | | | | | |
| 6-1 | MCC-Caf: 0.5 | 40 | 2.7 | 2.5 | 6.8 | Cloudy |
| | 004GOA: 0.6 | | | | | |
| | 022AOA: 0.4 | | | | | |
| 6-2 | MCC-Caf: 0.5 | 40 | 3.8 | 2.5 | 9.5 | Cloudy |
| | 004GOA: 0.6 | | | | | |
| | 022AOA: 0.4 | | | | | |
| 6-3 | MCC-Caf: 0.5 | 50 | 6.0 | 2.0 | 12.0 | Cloudy |
| | 004GOA: 0.6 | | | | | |
| | 022AOA: 0.4 | | | | | |
| 6-4 | MCC-Caf: 0.5 | 50 | 6.7 | 2.0 | 13.4 | Cloudy |
| | 004GOA: 0.6 | | | | | |
| | 022AOA: 0.4 | | | | | |
| 7-1 | MCC-Caf: 0.5 | 50 | 46.1 | 2.0 | 92.2*** | Clear |

Using a Cotton Ball as a Filter

| | | | | | | |
|---|---|---|---|---|---|---|
| 8-1 | MCC-Caf: 0.5 | 10 | 0.0 | NA | NA | NA |
| | 004GOA: 0.6 | | | | | |
| | 022AOA: 0.4 | | | | | |
| 8-2 | MCC-Caf: 0.5 | 10 + 10 + 20 | 0.0 | NA | NA | NA |
| | 004GOA: 0.6 | | | | | |
| | 022AOA: 0.4 | | | | | |
| 9-1 | MCC-Caf: 0.5 | 40 | 3.6 | 2.5 | 9.0 | Cloudy |
| | 004GOA: 0.6 | | | | | |
| | 022AOA: 0.4 | | | | | |
| 9-2 | MCC-Caf: 0.5 | 40 | 3.1 | 2.5 | 7.8 | Cloudy |
| | 004GOA: 0.6 | | | | | |
| | 022AOA: 0.4 | | | | | |
| 9-3 | MCC-Caf: 0.5 | 50 | 15.5 | 2.0 | 31.0 | Cloudy |
| | 004GOA: 0.6 | | (compressing | | | |
| | 022AOA: 0.4 | | the cotton) | | | |
| 9-4 | MCC-Caf: 0.5 | 50 | 12.6 | 2.0 | 25.2 | Cloudy |
| | 004GOA: 0.6 | | (compressing | | | |
| | 022AOA: 0.4 | | the cotton) | | | |
| 10-1 | MCC-Caf: 0.5 | 50 | 43.6 | 2.0 | 87.2⁽³⁾ | Clear |

*CaCl₂ containing formulation.
**Note: 004GOA: 0.3 grams/022AOA: 0.2 grams and 004GOA: 0.6 grams/022AOA: 0.4 grams represent a Carbopol/meglumine pellets ratio: 1.5; 004GOA: 0.7 grams/022AOA: 0.3 grams, a Carbopol/meglumine pellets ratio: 1.5.
***Obtained in less than one minute.

(b) Vodka as the Extraction Liquid

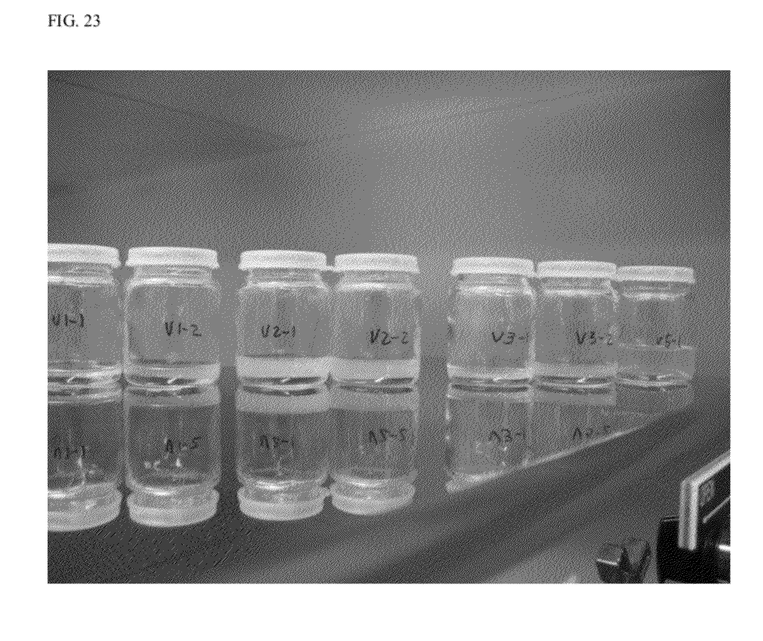

Results obtained using vodka as the extraction liquid is provided in Table 16 and FIGS. 23 (volume of vodka 10 mL) and 24 (volume of vodka 50 mL). For these tests, coffee filter paper was used as the filtering media. Mixtures not containing Carbopol/meglumine (Sample V5-1 and V6-1) were used as controls.

Samples having a ratio of Carbopol/meglumine of 0.3/0.2 or 0.6/0.4, exhibited no significant differences in filtrate weight and % caffeine recovery using 0.5 g (Sample V1) or 1.0 g (Sample V3) samples. The results showed that these ratios (Sample V1 and V4) were more effective for caffeine recovery (5 to 13% of recovery) than a ratio of 0.7/0.3 (Sample V2) that had about 30% recovery of caffeine.

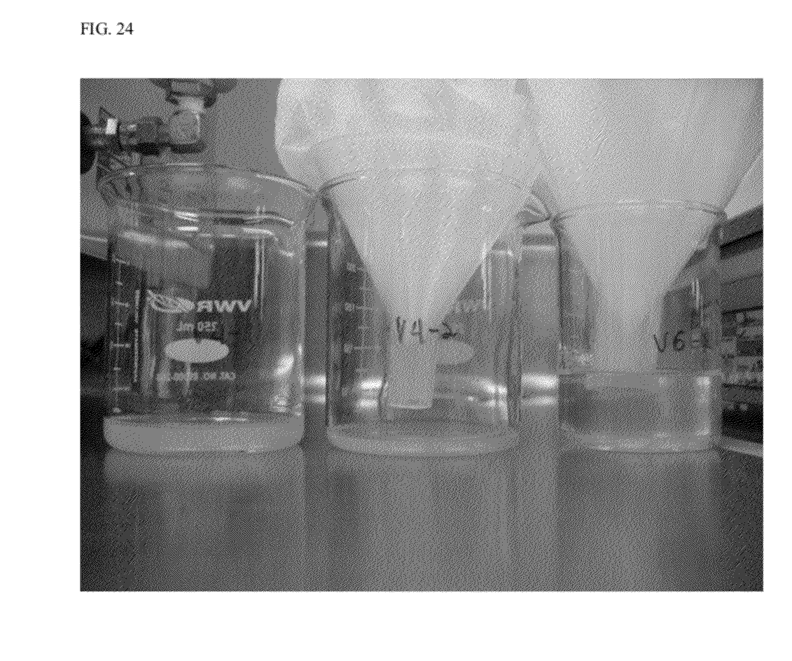

In general, after 10 minutes, between 5 and 30% of caffeine could be extracted with 10 mL of vodka (Samples V1-1 to V3-2), and between 12 and 31% of caffeine could be extracted with 50 mL of vodka (Samples V4-1 and V4-2). All filtrates from caffeine/Carbopol/meglumine mixtures (Samples V1 to V4) resulted in cloudy liquids (FIGS. 23-24). For the control mixtures (Samples V5-1 and V6-1), between 77 and 89% of the drug was recovered in a few minutes.

TABLE 18
Extraction Results for MCC-Caffeine with Carbopol* (11.1% w/w) and
Meglumine (17.4% w/w) System in Vodka.

| | | | | | | |
|---|---|---|---|---|---|---|
| | | | | Caffeine | Caffeine | |
| | | Vodka volume | Filtrate (g) | Theoretical | Recovery | |
| Sample | Solid Phase (g) | (mL) | after 10 min | (mg/mL) | (%) | Observations |

Using a coffee filter

| | | | | | | |
|---|---|---|---|---|---|---|
| V1-1 | MCC-Caf: 0.5 | 10 | 0.5 | 10.0 | 5.0 | Cloudy |
| | 004GOA: 0.3** | | | | | |
| | 022AOA: 0.2 | | | | | |
| V1-2 | MCC-Caf: 0.5 | 10 | 1.0 | 10.0 | 10.0 | Cloudy |
| | 004GOA: 0.3 | | | | | |
| | 022AOA: 0.2 | | | | | |
| V2-1 | MCC-Caf: 0.5 | 10 | 3.0 | 10.0 | 30.0 | Cloudy-white |
| | 004GOA: 0.7** | | | | | |
| | 022AOA: 0.3 | | | | | |
| V2-2 | MCC-Caf: 0.5 | 10 | 2.9 | 10.0 | 29.0 | Cloudy-white |
| | 004GOA: 0.7 | | | | | |
| | 022AOA: 0.3 | | | | | |
| V3-1 | MCC-Caf: 0.5 | 10 | 0.6 | 10.0 | 6.0 | Cloudy |
| | 004GOA: 0.6** | | | | | |
| | 022AOA: 0.4 | | | | | |
| V3-2 | MCC-Caf: 0.5 | 10 | 1.3 | 10.0 | 13.0 | Cloudy |
| | 004GOA: 0.6 | | | | | |
| | 022AOA: 0.4 | | | | | |
| V4-1 | MCC-Caf: 0.5 | 50 | 15.6 | 2.0 | 31.2 | Cloudy |
| | 004GOA: 0.7 | | | | | |
| | 022AOA: 0.3 | | | | | |
| V4-2 | MCC-Caf: 0.5 | 50 (10 + 10 + 30) | 5.8 | 2.0 | 11.6 | Cloudy |
| | 004GOA: 0.7 | | | | | |
| | 022AOA: 0.3 | | | | | |
| V5-1 | MCC-Caf: 0.5 | 10 | 7.7 | 10.0 | 77.0*** | Clear |
| V6-1 | MCC-Caf: 0.5 | 50 | 44.7 | 2.0 | 89.4*** | Cloudy |

*CaCl₂ containing formulation.
**Note: 004GOA: 0.3 grams/022AOA: 0.2 grams and 004GOA: 0.6 grams/022AOA: 0.4 grams represent a Carbopol/meglumine pellets ratio: 1.5; 004GOA: 0.7 grams/022AOA: 0.3 grams, a Carbopol/meglumine pellets ratio: 1.5.
***Obtained in less than 2-3 minutes.

### Example 8

### Carbopol/Meglumine Pellets Formulation

Carbopol pellets from formulation lot L066-01023 (MCC-101 (90%)/Carbopol 971P (10%) and water as granulating liquid) were mixed with meglumine pellets from formulation lot L066-01023 (MCC-101 (80%)/meglumine (20%)). About 200 g of this pellet mixture was coated with Opadry (5%)/Acryl-Eze (20%) system for a coat weight gain (WG) of about 3 and 5%, respectively (Table 19).

The test performed with 1.0 g of uncoated pellets from lots L066-01023 and L066-01022 (ratio 2.3) showed that the drug cannot be extracted with 10 mL of water (Table 12).

TABLE 19
Carbopol Pellets (70%) Meglumine Pellets (30%) Coated with Opadry
(Sub-Coating)/Acryl-Eze (Enteric Coating) System (L066-01022-
023OA)

| | | | | |
|---|---|---|---|---|
| Lot # | | | Pellets | |
| (L066) | Ingredients | g/batch | %/batch | % w/w after coating |

| | | | | |
|---|---|---|---|---|
| -01022 | MCC-101: 80% | 62 | 30 | MCC-101: 80.2 |
| | Meglumine: 20% | | | Carbopol 971: 6.4 |
| -01023 | MCC-101: 90% | 144 | 70 | Meglumine: 5.6 |
| | Carbopol 971: 10% | | | Opadry: 2.6 |
| | | | | Acryl-eze: 5.1 |

| | | | |
|---|---|---|---|
| Total | 206.0 | 100.0 | 100.0 |

### Example 9

### Characterization and Comparison of Filter Papers

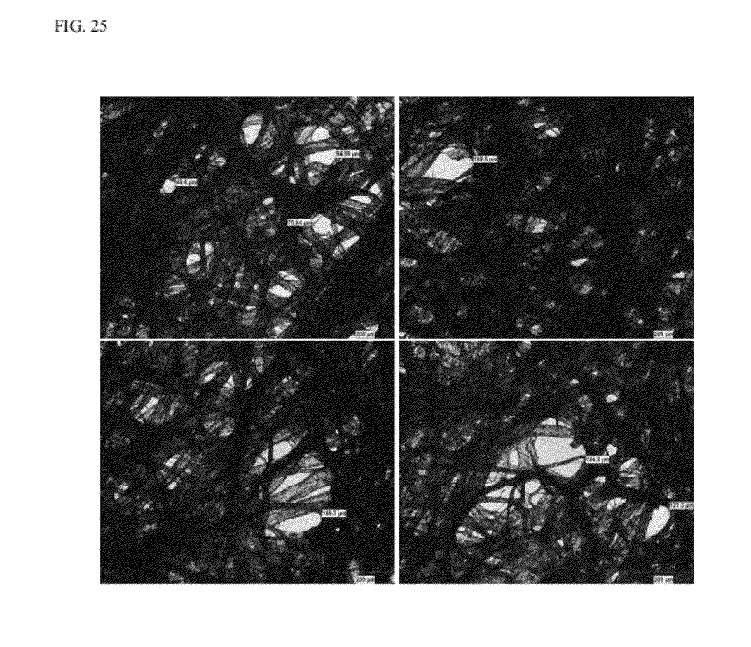

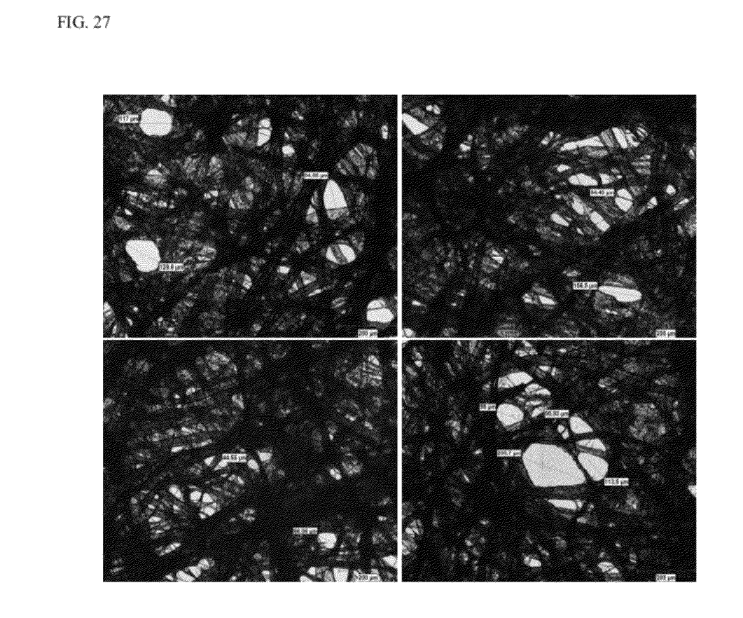

Life Brand™ coffee filter paper was used during the filtration tests. Three filters from this trademark and from “No Name” coffee filter were compared in Table 20 by optical microscopy (MO). The images (FIGS. 25-27) were taken at 100× magnification. The samples were taken (2.5 cm×1 cm) from different parts of the filter paper and the whole surface was examined.

Life Brand filters had a grammage of 29 g/m² and the largest pore sizes (longest length) observed were 160.5 185.5 and 217.9 gm. For “No name” filters, the grammage was 20-25 g/m² and the largest pore sizes were 206.6, 216.8 and 235.7 gm. Filters having different grammage (density of all types of paper expressed in terms of grams per square meter) and pores sizes could lead to a variation in the filtration rate.

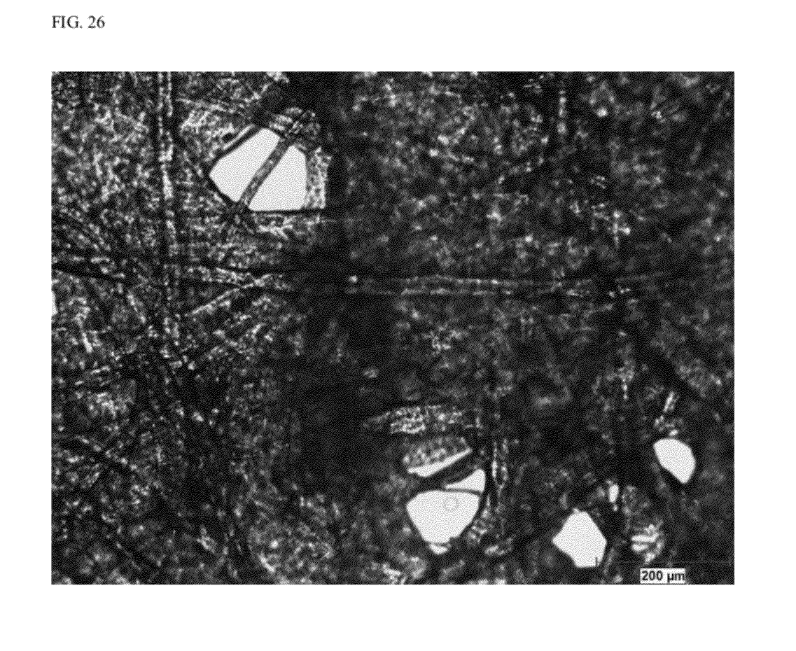

Wetting of the filter paper (FIG. 26) did not produce a significant difference in the sample.

TABLE 20
Paper Coffee Filters Comparison.

| Coffee Filters | Life Brand (8-12 cup basket) | | | No Name (8-12 cup basket) | | |
|---|---|---|---|---|---|---|
| Lot # | 1018311070 | | | 1029511020 | | |
| Filter # | 1 | 2 | 3 | 1 | 2 | 3 |
| Diameter (cm) | 20 | 20 | 20 | 22 | 22 | 23 |
| Thickness (μm) | 83 | 87 | 85 | 82 | 85 | 86 |
| Weight (g) | 0.9083 | 0.9355 | 0.9214 | 0.9335 | 0.9454 | 0.9457 |
| Grammage (g/m²) | 28.9 | 29.8 | 29.3 | 24.6 | 24.9 | 20.9 |
| Pore longest length | 185.5 | 217.9 | 160.5 | 235.7 | 216.8 | 206.6 |
| (μm) | | | | | | |

### Example 10

### Extraction from Tablet Formulations

The following tablet formulations (shown in Table 21) comprised enteric-coated pellets containing 25% (w/w of a drug-HCl) pellets and 25% Carbopol/meglumine pellets (0.7/0.3). As an external phase, microcrystalline cellulose (Tabulose-102) was combined with Carbopol (71G granules or 971P powder), meglumine powder and magnesium stearate as lubricant for tableting.

Carbomers can be used as tablet binder at the concentrations between 5-10% (see, e.g., Rowe R C, Sheskey P J, Owen S C, eds. Handbook of Pharmaceutical Excipients. 5th ed., 2006) (“Rowe”). As per “Guidance Document for Processing Carbopol® Polymers in Oral Solid Dosage Forms” (see Lubrizol website), 10-30% of Carbopol 71G (granular form) can be included in direct compressible formulations and a maximum 5% for powder grades. Carbopol is soluble in water and after neutralization in 95% alcohol. Agents that may be used to neutralize include amino acids, sodium bicarbonate, and polar organic amines. The more viscous aqueous gels are achieved at pH 6-11. The viscosity is considerably reduced at pH values less than 3 or greater than 12, or in the presence of strong electrolytes (see Rowe).

TABLE 21
Carbopol/Meglumine Pellets-Powder Tablet Formulations.

| | | For- | | |
|---|---|---|---|---|
| | | mulation | Formulation | Formulation |
| | | Lot: L066- | Lot: L066- | Lot: L066- |
| Ingredient | | 01025 | 01026 | 01027 |
| name | Ingredients Lot | mg/unit | mg/unit | mg/unit |
| Drug coated | L107-03025- | 150.0 | 0 | 150.0 |
| pellets | 5O20A | | | |
| Carbopol 971P | L066- | 105.0 | 105.0 | 105.0 |
| coated pellets | 01004GOA | | | |
| Meglumine | L066- | 45.0 | 45.0 | 45.0 |
| coated pellets | 01022AOA | | | |
| MCC-102 | C00006 | 264.0 | 254.0 | 254.0 |
| Carbopol 71G | TW7XGAJ107 | 30.0 | 0.0 | 0.0 |
| Carbopol 971P | 0100825297 | 0.0 | 30.0 | 30.0 |
| powder | | | | |
| Meglumine | 20716960 | 0.0 | 10.0 | 10.0 |
| powder | | | | |
| Magnesium | C00007 | 6.0 | 6.0 | 6.0 |
| stearate | | | | |

| | | | |
|---|---|---|---|
| Core Total | 600.0 | 450.0 | 600.0 |

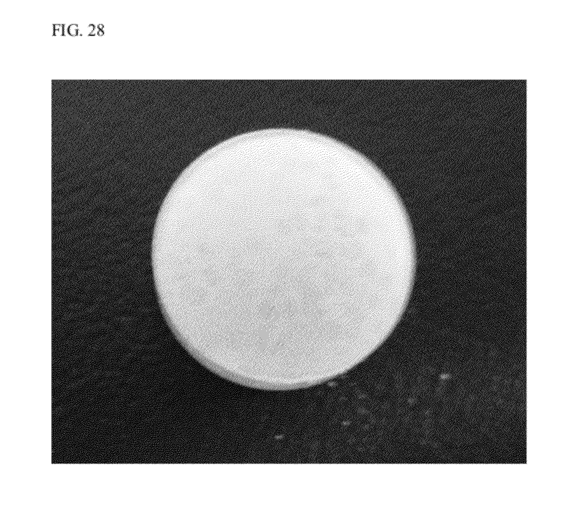

Tablets containing 150 and 300 mg of pellets were compressed (Table 22 and FIG. 28). For this study 150 mg of enteric-coated pellets, prepared from an X-HCl drug, were used. Compressing at 2000 lbs produced 12 mm round biconvex tablets with hardness of 4 kp.

TABLE 22
Tableting Results Using 12 mm Tooling.

| | | | Hardness |
|---|---|---|---|
| Sample | Compression Force (lbs) | Tablet Weight (mg) | (kp) |
| From lot | 2000 | 601.2 | 4.3 |
| L066-01025 | | 602.1 | 4.4 |

Filtration testing results are shown in Table 23. Two tablets and the solvent were crushed until complete disintegration of pellets. An additional amount of solvent was added to the slurry that was retained over the filter.

Testing of the formulation containing 5% of Carbopol showed that powder grade (lot L066-01026) is more efficient than the granular grade (lot L066-01025) due to the larger surface area, or possible due to the presence of meglumine. Greater volumes of solvent led to very low drug concentration. Carbopol and meglumine are also soluble in water.

TABLE 23
Extraction Results for Carbopol Pellets/Powder and Meglumine Tablets.

| | | | |
|---|---|---|---|
| | | | Filtrate |
| Sample | Solid Phase (g) | Solvent volume (mL) | (g) after 10 min |

Using a coffee filter

| | | | |
|---|---|---|---|
| T1-1 | 2 Tablets from lot | Water: 10 | 3.3 |
| T1-2 | L066-01025 | Water: 20 (10 + 10) | 12.7 |
| T1-3 | | Vodka: 10 | 5.1 |
| T2-1 | 2 Tablets from lot | Water: 10 | 0.0 |
| T2-2 | L066-01026 | Water: 20 (10 + 10) | 0.0 |
| T2-3 | | Water: 30 | 0.5 |
| | | (10 + 10 + 10) | |
| T3-1 | 2 Tablets from lot | Water: 30 | 2.1 |
| T3-2 | L066-01026 | Water: 50 (30 + 20) | 12.1 |

Tables 24 to 26 show additional formulations and process parameters for lots prepared. For meglumine pellets formulation (lot #L066-01028), PVP was added as a binder in order to improve yield and quality of pellets.

TABLE 24
Lot L066-01028 Formula Description.

| Ingredients | Lot # | %* | g/batch |
|---|---|---|---|
| MCC-101 | C00021 | 77.5 | 155.0 |
| Meglumine | C00063 | 20.0 | 40.0 |
| Plasdone K-29/32** | C00033 | 2.5 | 5.0 |
| Water (Purified water)*** | 2011JN28 | 55.0 | 110.0 |

| | | |
|---|---|---|
| Total | 100.0 | 200.0 |

*% w/w on dry basis;
**dissolved in granulation water;
***% w/w on dry basis, not calculated in the total since will be removed during the drying process.

TABLE 25
Lot L066-01029 Formula Description.

| Ingredients | Lot # | %* | g/batch |
|---|---|---|---|
| MCC-101 | C00021 | 90.0 | 180.0 |
| Carbopol 971 | C00051 | 10.0 | 20.0 |
| Water (Purified water)** | 2011JN28 | 45.0 | 90.0 |

| | | |
|---|---|---|
| Total | 100.0 | 200.0 |

*% w/w on dry basis;
**% w/w on dry basis, not calculated in the total since will be removed during the drying process.

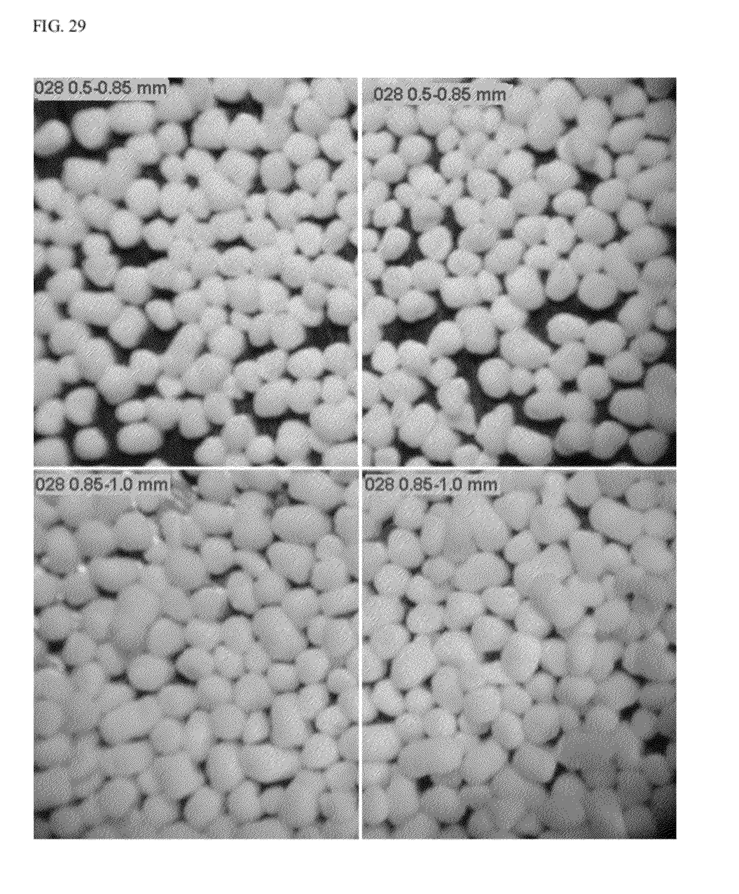

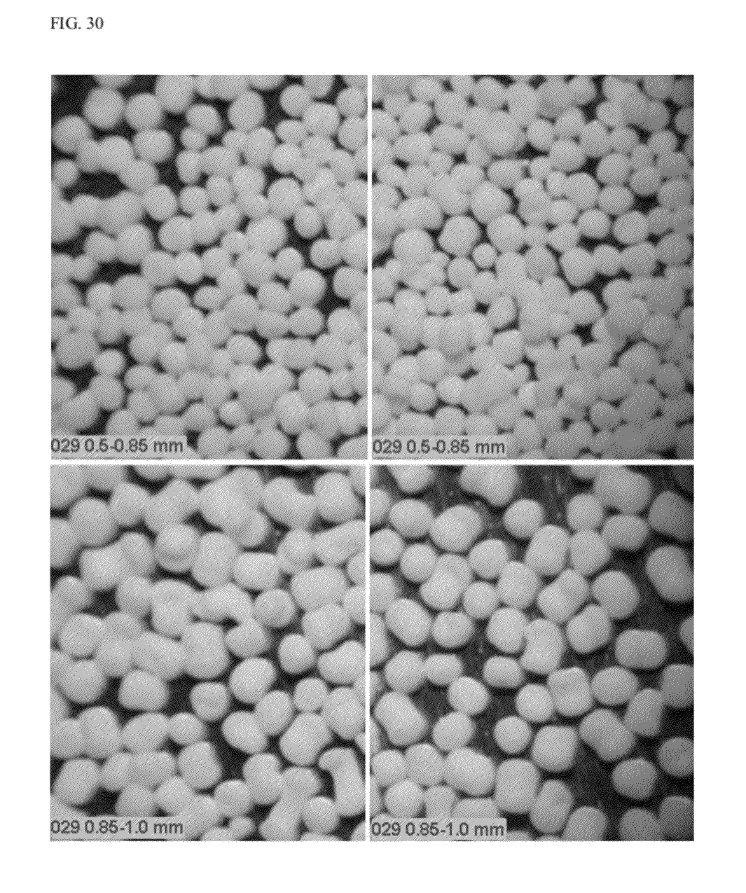

TABLE 26
Lot L066-01028B and Lot L066-01029 Process Parameters.

| Step | L066-01028 | Lot L066-01029 |
|---|---|---|
| Sieving/ | 30 mesh/2 min at 60 RPM | 30 mesh/2 min at 60 RPM |
| Dry mixing | | |
| Low Shear | Liquid pump rate: 37 g/min | Liquid pump rate: 15 g/min |
| Granulation | Total wetting time: 3.5 min | Total wetting time: 10 min |
| | Mixing speed: 60 RPM | Mixing speed: 60 RPM |
| Extrusion | Dome: 1.0 mm | Dome: 1.0 mm |
| | Shaft speed: 50 RPM | Shaft speed: 30 RPM |
| | Load: 2.5 Ap | Load: 2.8 Ap |
| | Extrusion time: 2.5 min | Extrusion time: 4.0 min |
| Spheronization | Plate speed: | Plate speed: |
| | 750/500 RPM | 960/1750/500 RPM |
| | Spheronization | Spheronization |
| | time: 4/4 min | time: 10/10/5 min |
| | Pellets and rods | Pellets, rods and dumbbell |
| | (see FIG. 29) | shape pellets (see FIG. 30) |
| Drying | Temperature: 50° C. | Temperature: 50° C. |
| (on trays | Drying time: 5 h | Drying time: 2 h |
| in an oven) | LOD after 72 h at RT: 6% | LOD after 72 h at RT: 6% |

Produced pellets were evaluated in terms of shape (FIGS. 29-30), yields, pore size distributions (PSD) and density (Table 27).

Consistent with previous data, both lots showed presence of pellets and rods and for lot L066-01029 (Carbopol pellets) dumbbell shaped pellets were observed, notably in the fractions retained in 1.18 mm (16 mesh) and 1.0 mm (18 mesh) sieves.

As shown in Table 27, 66% of dry material from lot L066-01028 (meglumine (MGL) formulation)) resulted in pellets having between 0.5 and 1.0 mm of size. Carbopol (CPL) formulation produced larger pellets, as only 46.8% had sizes between 0.5 and 1.0 mm.

Both lots showed similar bulk density, about 0.7 g/cm³.

TABLE 27
Pelletization Results.

| | PSD | | | | | |
|---|---|---|---|---|---|---|
| Pellet | Sieve | Opening | Retained | Pelletization | Pelletization | Density_{0.5-1.0} |
| Formulation | (mesh) | (mm) | (%) | Yield tot. (%) | Yield_{0.5-1.0} (%) | (g/cm³) |
| L066-01028 | 16 | 1.18 | 0 | 72.3 | 66.2 | 0.683 |
| (MGL) | 18 | 1.0 | 1.6 | | | |
| | 20 | 0.85 | 68.2 | | | |
| | 35 | 0.5 | 25.5 | | | |
| | Pan | — | 4.9 | | | |
| L066-01029 | 16 | 1.18 | 13.0 | 95.8 | 46.8 | 0.700 |
| (CPL) | 18 | 1.0 | 36.3 | | | |
| | 20 | 0.85 | 30.5 | | | |
| | 35 | 0.5 | 19.2 | | | |
| | Pan | — | 1.0 | | | |

Retained (%): Amount of total pellets retained/amount of total pellets.
Yield tot. (%): Calculated as amount of total of pellets/amount total of dry blend.
Yield_{0.5-1.0} (%): Calculated as pellets load fraction 0.5-1.0 mm/amount of total dry blend.

The filtration/extraction testing was carried out as discussed previously, with 0.5 g of a mixture containing 20% of caffeine as the drug model. The total amount of caffeine available is 100 mg, which is the equivalent to 3 to 4 MoxDuo 30 mg dose tablets (150 mg×0.2=30 mg). The extraction with 10 mL produced a solution containing about 10 mg/mL. The extraction results are provided in Table 28.

TABLE 28
Extraction Results for MCC-Caffeine (100 mg) with Carbopol (10% w/w)
and Meglumine (20% w/w) Uncoated pellets.

| | | | | | |
|---|---|---|---|---|---|
| | | | Filtrate | | |
| | | Solvent | (g) | Caffeine | Caffeine |
| | | volume | after | Theoretical | Recovery |
| Sample | Solid Phase (g) | (mL) | 10 min | (mg/mL) | (%) |

Using a coffee filter

| | | | | | |
|---|---|---|---|---|---|
| 1 | CPL 029: 0.3 | Water: 10 | 0 | — | — |
| | MGL 028: 0.2 | | | | |
| 2-1 | MCC-Caf: 0.5 | Water: 10 | 1.7 | 10.0 | 17.0 |
| | CPL 029: 0.3 | | | | |
| | MGL 028: 0.2 | | | | |
| 2-2 | MCC-Caf: 0.5 | Water: 10 | 1.6 | 10.0 | 16.0 |
| | CPL 029: 0.3 | | | | |
| | MGL 028: 0.2 | | | | |
| 3 | MCC-Caf: 0.5 | Water: 10 | 0 | — | — |
| | CPL 029: 0.6 | | | | |
| | MGL 028: 0.4 | | | | |
| 4 | MCC-Caf: 0.5 | Water: | 1.5 | 5.0 | 7.5 |
| | CPL 029: 0.6 | 10 + 10 | | | |
| | MGL 028: 0.4 | | | | |
| 5 | MCC-Caf: 0.5 | Water: 10 | 0 | — | — |
| | CPL 029: 0.5 | | | | |
| | MGL 028: 0.33 | | | | |
| 6 | MCC-Caf: 0.5 | Water: | 1.5 | 5.0 | 7.5 |
| | CPL 029: 0.5 | 10 + 10 | | | |
| | MGL 028: 0.33 | | | | |
| 7 | MCC-Caf: 0.5 | Vodka: 10 | 0 | — | — |
| | CPL 029: 0.5 | | | | |
| | MGL 028: 0.33 | | | | |
| 8 | MCC-Caf: 0.5 | Vodka: | 0.4 | 5.0 | 2.0 |
| | CPL 029: 0.5 | 10 + 10 | | | |
| | MGL 028: 0.33 | | | | |

Tables 29 and 30 summarize and compare the different Carbopol/alkalining agent pellets formulations used in this study.

TABLE 29
Formulations of Carbopol and Alkalining Agents.

| | Lot | Carbopol | MCC- | | | Granulation |
|---|---|---|---|---|---|---|
| System | (L066) | 971P | 101 | Lactose | Other | liquid |
| Carbopol/ | -01013 | 11 | 70 | — | Talc: 18 | Water/Ethanol |
| NaHCO₃ | | | | | NaHCO₃: 1 | |
| Carbopol/ | -01004G | 13 | 50 | 30.0 | Talc: 6.0 | Water/CaCl₂ |
| Meglumine | -01022A | — | 80 | — | Meglumine: 20 | Water |
| Carbopol/ | -01023 | 10 | 90 | — | — | Water |
| Meglumine | -01022A | — | 80 | — | Meglumine: 20 | Water |
| Carbopol/ | -01029 | 10 | 90 | — | — | Water |
| Meglumine | -01028 | — | 77.5 | — | Meglumine: 20 | Water/PVP |

TABLE 30
Carbopol and Alkalining 100 mg Caffeine Filtration/Extraction Results.

| | Lot | Amount of | | Solvent | | Recovery |
|---|---|---|---|---|---|---|
| System | (L066) | pellets (g) | Conditions | 10 mL | Filtrate (g) | (%) |
| Carbopol less | -01013 | 0.5 | T = 0 | Water | — | 0 |
| than 11%/ | | 0.5 | T = 0 | Vodka | 0.3 | 3 |
| NaHCO₃ | | 0.5 | 12 weeks | Water | 0.1 | 1 |
| Coated pellets | | | 25° C./60% RH | | | |
| | | 0.5 | 12 weeks | Vodka | 0 | 0 |
| | | | 25° C./60% RH | | | |
| | | 0.5 | 12 weeks | Water | 0.4 | 4 |
| | | | 40° C./75% RH | | | |
| | | 0.5 | 12 weeks | Vodka | 0.1 | 1 |
| | | | 40° C./75% RH | | | |
| Carbopol less | -01004G/ | 0.3/0.2 | T = 0 | Water | 0.3 | 3 |
| than 13%/ | -01022A | | | | | |
| Meglumine | -01004G/ | 0.3/0.2 | T = 0 | Vodka | Not tested | NA |
| Coated pellets | -01022A | | | | | |
| | -01004G/ | 0.3/0.2 | 4 weeks | Water | 0.7 | 7 |
| | -01022A | | 40° C./75% RH | | | |
| | -01004G/ | 0.5/0.3 | 4 weeks | Water | 1.0 | 10 |
| | -01022A | | 40° C./75% RH | | | |
| | -01004G/ | 0.3/0.2 | 4 weeks | Vodka | 3.1 | 28 |
| | -01022A | | 40° C./75% RH | | | |
| | -01004G/ | 0.3/0.2 | 12 weeks | Water | 2.4 | 22 |
| | -01022A | | 25° C./60% RH | | | |
| | -01004G/ | 0.3/0.2 | 12 weeks | Vodka | 0.9 | 9 |
| | -01022A | | 25° C./60% RH | | | |
| | -01004G/ | 0.3/0.2 | 12 weeks | Water | 5.0 | 48 |
| | -01022A | | 40° C./75% RH | | | |
| | -01004G/ | 0.3/0.2 | 12 weeks | Vodka | 4.3 | 39 |
| | -01022A | | 40° C./75% RH | | | |
| Carbopol 10%/ | -01023/ | 0.7/0.3 | T = 0 | Water | 0 | 0 |
| Meglumine | -01022B | | | | | |
| Uncoated | -01023/ | 0.7/0.3 | T = 0 | Vodka | Not tested | NA |
| pellets | -01022B | | | | | |
| Carbopol 10%/ | -01029/ | 0.5/0.33 | T = 0 | Water | 0 | 0 |
| Meglumine | -01028 | | | | | |
| Uncoated | -01029/ | 0.5/0.33 | T = 0 | Vodka | 0 | 0 |
| pellets | -01028 | | | | | |

For Carbopol pellets not containing CaCl₂ (lots L066-01013, -01023 and -01029), 0.5 grams of pellets were able to prevent the extraction of caffeine from the same amount of material (0.5 g of caffeine-MCC mix). For Carbopol pellets containing CaCl₂ (lot L066-01004) increasing the amount of pellets from 0.3 (7% of caffeine recovered) to 0.5 (10% of caffeine recovered) increased the amount of this electrolyte and, as a result, decreased the viscosity of Carbopol.

### Example 12

### Immediate Release Tablets

This study investigated immediate release tablet formulations that comprised either Carbopol/meglumine pellets or Carbopol and meglumine powders. The materials used in the formulations are provided in Table 31.

TABLE 31
Materials for Immediate Release Tablet Formulations.

Materials

| | |
|---|---|
| Ingredients | Caffeine USP |
| | Magnesium stearate |
| | Meglumine |
| | Microcrystalline cellulose AVICEL 102 |
| | Cross linked polyacrylic acid (Carbopol 971P) |
| | Colloidal Silicon Dioxide (AEROSIL 200) |
| | Pregelatinized starch (Starch 1500) |
| Solvents for extraction | Tapped water |
| proposal | Vodka (Moskovskaya) 40% |

Two powder Carbopol/meglumine pellet formulations, lots L066-01035A and -01035B, and two powder Carbopol/meglumine powder formulations, lots L066-01036A and -01036B, were developed, comprising the ingredients provided in Tables 32-35.

TABLE 32
Ingredients for Formulation L066-01035A (5 mg).

| Ingredient | % | mg/unit | g/batch |
|---|---|---|---|
| Caffeine USP | 2.50% | 5.0 | 0.125 |
| Carbopol/Meglumine | 28.50% | 57.0 | 1.425 |
| pellets | | | |
| Pregelatinized Corn Starch | 43.00% | 86.0 | 2.150 |
| MCC-102 | 24.50% | 49.0 | 1.225 |
| Colloidal Silicon Dioxide | 0.75% | 1.5 | 0.038 |
| Magnesium Stearate | 0.75% | 1.5 | 0.038 |
| Core Total | 100.00% | 200.0 | 5.00 |

TABLE 33
Ingredients for Formulation L066-01035B (20 mg).

| Ingredient | % | mg/unit | g/batch |
|---|---|---|---|
| Caffeine USP | 10.00% | 20.0 | 0.500 |
| Carbopol/Meglumine | 28.50% | 57.0 | 1.425 |
| pellets | | | |
| Pregelatinized Corn Starch | 38.50% | 77.0 | 1.925 |
| MCC-102 | 21.50% | 43.0 | 1.075 |
| Colloidal Silicon Dioxide | 0.75% | 1.5 | 0.038 |
| Magnesium Stearate | 0.75% | 1.5 | 0.038 |
| Core Total | 100.00% | 200.0 | 5.00 |

TABLE 34
Ingredients for Formulation L066-01036A (5 mg).

| Ingredient | % | mg/unit | g/batch |
|---|---|---|---|
| Caffeine USP | 3.50% | 5.0 | 0.175 |
| Carbopol 971P | 7.00% | 10.0 | 0.350 |
| Meglumine | 2.00% | 2.9 | 0.100 |
| Pregelatinized Corn Starch | 56.00% | 80.4 | 2.800 |
| MCC-102 | 29.50% | 42.3 | 1.475 |
| Colloidal Silicon Dioxide | 1.00% | 1.4 | 0.050 |
| Magnesium Stearate | 1.00% | 1.4 | 0.050 |
| Core Total | 100.00% | 143.5 | 5.000 |

TABLE 35
Ingredients for Formulation L066-01036B (20 mg).

| Ingredient | % | mg/unit | g/batch |
|---|---|---|---|
| Caffeine USP | 14.00% | 20.0 | 0.700 |
| Carbopol 971P | 7.00% | 10.0 | 0.350 |
| Meglumine | 2.00% | 2.9 | 0.100 |
| Pregelatinized Corn Starch | 51.00% | 73.2 | 2.550 |
| MCC-102 | 24.00% | 34.4 | 1.200 |
| Colloidal Silicon Dioxide | 1.00% | 1.4 | 0.050 |
| Magnesium Stearate | 1.00% | 1.4 | 0.050 |
| Core Total | 100.00% | 143.5 | 5.00 |

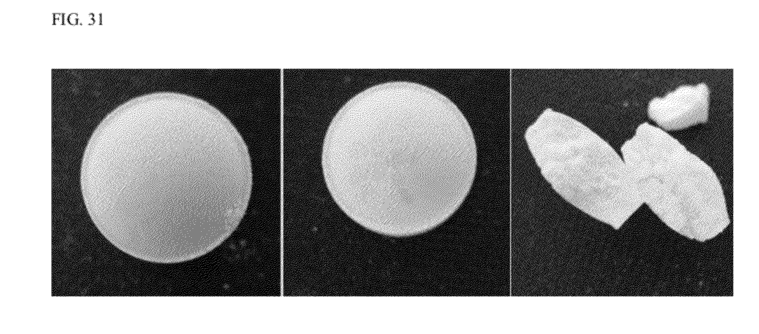

Tablets were produced using a Hydraulic Press (Model C, Carver Inc.) with 8 mm diameter standard concave tooling and a compression force of 1000-1500 lbf (2-3 kp). Images of filtration testing were taken using a Canon PowerShot A640 digital camera (FIG. 31).

Powder or crushed tablets were transferred into a mortar and 10 mL of solvent at room temperature was added. The pellets mixtures were vigorously grinded using a mortar and pestle until all pellets were completely destroyed.

The resulting suspensions were immediately filtered through a standard coffee filter. Viscosity increases were evaluated visually. Filtration rates were evaluated by comparing the amount of filtered liquid phase recovery after 10 minutes to the initial 10 mL.

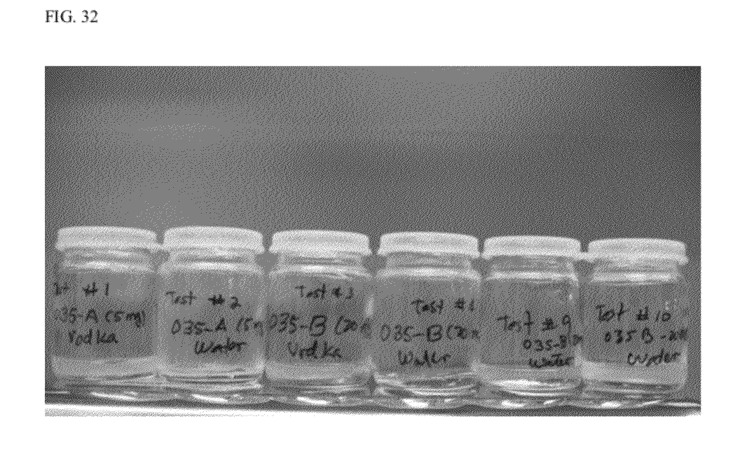

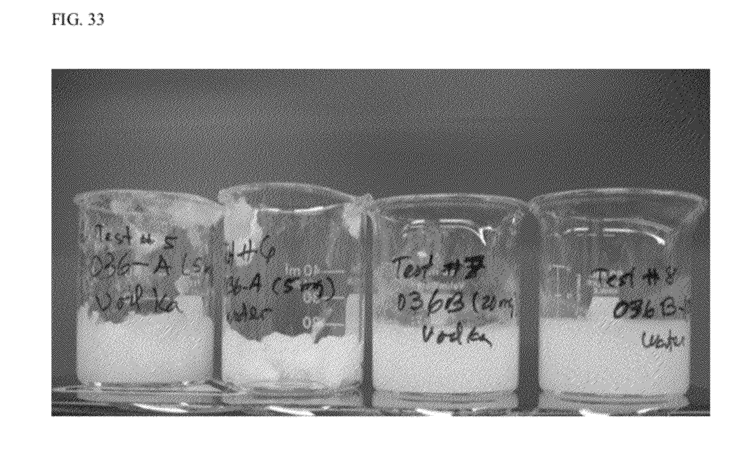

The amount of Carbopol and meglumine used in the two comparative tablet formulations (powder versus pellet) was kept constant. It is evident from Table 36 that the direct use of powdered Carbopol and meglumine (lots L066-01036A and -01036B) restricts any filtration of the resulting aqueous or aqueous alcohol solvent extract of the tablet (compare FIGS. 32 and 33). Thus the use of powdered hydrophilic polymers may detrimentally affect the immediate release aspect of the product.

TABLE 36
Carbopol and Meglumine Formulations Filtration/Extraction Results.

| | | | | Filtrate | Caffeine |
|---|---|---|---|---|---|
| Lot | | # of | | wt (g) after | recovered |
| (L066) | Test # | Tablets | Solvent | 10 min | (mg/mL) |
| −01035A | 1, 2 | 10 | Water: 10 ml | 0.1 cloudy | 5.0 |
| (5 mg) | | | Vodka: 10 ml | 1.5 cloudy | 7.5 |
| −01035B | 10 | 3 | Water: 10 ml | 2.0 cloudy | 6.0 |
| (20 mg) | | | Vodka: 10 ml | ND | NA |
| −01035B | 3, 4, 9 | 5 | Water: 10 ml | 0.0 | NA |
| (20 mg) | | | Water: 10 ml | 1.2 cloudy | 12.0 |
| | | | Vodka: 10 ml | 0.7 cloudy | 7.0 |
| −01036A | 5, 6 | 10 | Water: 10 ml | 0 | NA |
| (5 mg) | | | Vodka: 10 ml | 0 | NA |
| | | | Vodka: 20 ml | 0 | NA |
| −01036B | 7, 8 | 5 | Water: 10 ml | 0 | NA |
| (20 mg) | | | Water: 30 ml | 0 | NA |
| | | | Vodka: 10 ml | 0 | NA |
| | | | Vodka: 20 ml | 0 | NA |

Dissolution testing was performed using the parameters as shown in Table 37. The results, provided in Table 38, indicated that rapid dissolution of the pellet formulation of lots L066-01035A and -01035B is not affected, whereas similar aqueous or aqueous alcohol solvent extract of this tablet still restricts complete recovery of the active ingredient (Table 36), thereby deterring the ability to recover the complete dose when the tablet is manually manipulated for ulterior motives.

TABLE 37
Dissolution Testing Parameters.

| | |
|---|---|
| Samples | Method |

| | | | |
|---|---|---|---|
| Initial volume (mL) | 900 | Apparatus | II |
| Volume of aliquots (mL) | 2 | Speed (rpm) | 50 |
| Label claim (mg/tablet) | 20 | Disso. | 0.1N HCl |
| | | medium | |
| Dilution factor | 1 | Surfactant(s) | N/A |

TABLE 38
Dissolution Rate of Carbopol and Meglumine Tablet Formulations.

| | Sampling | | | |
|---|---|---|---|---|
| Lot | time | Vessels # | % Dissolution | % RSD |
| L066-01036B | 5 min | 1 | 5 | 1.3 |
| (Carbopol powder) | | 2 | 5 | |
| | | 3 | 5 | |
| | | Average | 5 | |
| | 10 min | 4 | 8 | 0.9 |
| | | 5 | 8 | |
| | | 6 | 8 | |
| | | Average | 8 | |
| | 15 min | 1 | 11 | 1.7 |
| | | 2 | 11 | |
| | | 3 | 11 | |
| | | Average | 11 | |
| | 30 min | 4 | 20 | 8.9 |
| | | 5 | 21 | |
| | | 6 | 18 | |
| | | Average | 20 | |
| L066-01035B | 5 min | 1 | 92 | 1.7 |
| (Carbopol pellets) | | 2 | 95 | |
| | | 3 | 94 | |
| | | Average | 94 | |
| | 10 min | 1 | 93 | 2.1 |
| | | 2 | 97 | |
| | | 3 | 95 | |
| | | Average | 95 | |
| | 15 min | 1 | 94 | 2.8 |
| | | 2 | 99 | |
| | | 3 | 95 | |
| | | Average | 96 | |
| | 30 min | 1 | 94 | 2.0 |
| | | 2 | 98 | |
| | | 3 | 96 | |
| | | Average | 96 | |

% RSD = % relative standard deviation.

### Example 13

### Morphine/Oxycodone Controlled Release Tablets with Abuse Deterrent Pellets

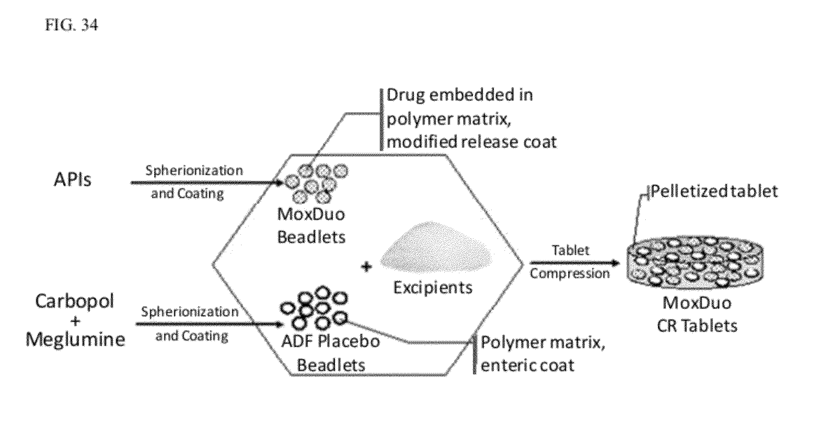

The morphine/oxycodone controlled release (CR) tablet with abuse deterrent pellets (“CR/AD tablets”) were produced from a dry blend of excipients, multiparticulate hydrophilic polymer abuse deterrent pellets, and multiparticulate modified release pellets containing morphine sulfate and oxycodone hydrochloride in a fixed 3:2 ratio. This dry blend is compressed into oral tablets, as shown in FIG. 34, using a standard, gravity-feed, pharmaceutical tableting machine.

The composition of the CR/AD tablets is provided in Table 39, while the composition of the abuse deterrent pellets is provided in Table 40.

TABLE 39
Composition of Morphine/Oxycodone CR Tablet.

| | | Test Product | |
|---|---|---|---|
| | | Quantity | |
| | | (mg) per | |
| Component | Function | tablet | % |
| Opioid Modified Release | MR Beadlet | 180.0 | 22.5 |
| Beadlet (Dual opioid) | | | |
| Contains Morphine Sulfate | API | 30.0 | 3.75 |
| Contains Oxycodone HCl | API | 20.0 | 2.5 |
| Abuse Deterrent Pellet | Filler/Diluent | 180.0 | 22.5 |
| | Abuse Deterrent | | |
| | Feature | | |
| Microcrystalline Cellulose, | Filler/Diluent | 317.2 | 39.7 |
| PH102 | | | |
| Microcrystalline Cellulose, | Filler/Diluent | 79.3 | 9.9 |
| PH200 | | | |
| Carbopol 971P | Hydrophilic polymer | 18.1 | 2.3 |
| Croscarmellose Sodium | Disintegrant | 22.7 | 2.8 |
| Magnesium Stearate | Lubricant | 2.7 | 0.3 |
| Total | | 800.0 | 100 |

TABLE 40
Composition of Abuse Deterrent Pellets.

| | | | ADF Excipient** | |
|---|---|---|---|---|
| | | | Quantity (g) | |
| | Component | Function | per batch | % |
| Carbopol | Carbopol 971P | Hydrophilic | 300.0 | 10.0 |
| Beadlet | | polymer | | |
| Core | Microcrystalline | Filler/Diluent | 2700.0 | 90.0 |
| | Cellulose, PH101 | | | |
| | Purified Water* | Process Aid | — | — |
| | Sub Total | | 3000.0 | 100 |
| Enteric Coat | Carbopol Beadlet | | (3000.0) | 82.8 |
| | Opadry 20A19301 | Sealer | 150.0 | 4.1 |
| | Purified Water* | Process Aid | | |
| | Methacrylic Acid, | Film Forming | 290.7 | 8.1 |
| | Copolymer | Agent | | |
| | Dispersion | | | |
| | Eudragit | | | |
| | L30-D55*** | | | |
| | Triethyl Citrate | Plasticizer | 36.4 | 1.0 |
| | Talc (197) | Antitacking | 145.4 | 4.0 |
| | | Agent | | |
| | Purified Water* | Process Aid | | |
| | Sub Total | | 3622.5 | 100 |
| Meglumine | Meglumine | pH modifier | 720.0 | 20.0 |
| Beadlet Core | Plasdone K-29/32 | Filler/Diluent | 90.0 | 2.5 |
| | Microcrystalline | Filler/Diluent | 2790.0 | 77.5 |
| | Cellulose, PH101 | | | |
| | Purified Water* | Process Aid | | |
| | Sub Total | | 3600.0 | 100 |
| Enteric Coat | Meglumine Beadlet | | (3600.0) | 82.8 |
| | Opadry 20A19301 | Sealer | 180.0 | 4.1 |
| | Purified Water* | Process Aid | | |
| | Methacrylic Acid, | Film Forming | 350.2 | 8.1 |
| | Copolymer | Agent | | |
| | Dispersion | | | |
| | Eudragit | | | |
| | L30-D55*** | | | |
| | Triethyl Citrate | Plasticizer | 43.6 | 1.0 |
| | Talc (197) | Antitacking | 173.2 | 4.0 |
| | | Agent | | |
| | Purified Water* | Process Aid | | |
| | Sub Total | | 4347.0 | 100 |
| Abuse | Carbopol Beadlets | ADF | 9800 | 70.0 |
| Deterrent | Meglumine Beadlets | pH modifier | 4200 | 30.0 |
| Pellet | Total | | 14000 | 100 |

*Removed during drying process via evaporation.
**ADF = abuse deterrent formulation.
***Expressed as the 30% solids, remaining water removed during drying processing step via evaporation. Enteric coating nominal weight gain is 15%.

Filtration and extraction testing was performed on the CR/AD tablets and the results were compared to filtration extraction test results of commercially available OxyContin® 20 mg CR Tablets.

The CR/AD tablets were produced using a Piccola (Riva, SA) rotary tablet press with oval standard concave B tooling with a resulting tablet hardness of 10-20 kP.

Tablets were transferred to a mortar and pestle and 10 mL of water or 10 mL of aqueous alcohol (40% v/v to approximate vodka) at a temperature between 26 and 28° C. was added. The tablets were crushed, and the resulting mixtures were shaken for 10 minutes and then filtered through a coffee filter. Viscosity increase was evaluated visually, while filtration rate was evaluated by comparing the amount of liquid added in relation to amount the filtrate phase recovered after 10 minutes. The process was repeated for increasing amounts of solvent, 20 mL, 30 mL, 40 mL and 50 mL. The filtration testing results are presented in Tables 41 (water as the solvent) and 42 (40% alcohol as the solvent).

TABLE 41
Filtration Testing Results of CR/AD Tablet and
CR OxyContin Tablet Using Water as the Solvent.

| Solvent | | | | Volume Recovery |
|---|---|---|---|---|
| Volume | Tablet | Trials | Volume (mL) | (%) |
| 10 mL | CR/AD | 1 | 1.2898 | 9.4 |
| | | 2 | 0.5826 | |
| | OxyContin | 1 | 8.6838 | 85.8 |
| | | 2 | 8.4675 | |
| 20 mL | CR/AD | 1 | 3.6186 | 18.4 |
| | | 2 | 3.7290 | |
| | OxyContin | 1 | 18.3987 | 90.8 |
| | | 2 | 17.9102 | |
| 30 mL | CR/AD | 1 | 5.4662 | 18.1 |
| | | 2 | 5.3771 | |
| | OxyContin | 1 | 28.1897 | 93.2 |
| | | 2 | 27.7224 | |
| 40 mL | CR/AD | 1 | 12.1984 | 28.1 |
| | | 2 | 10.3109 | |
| | OxyContin | 1 | 37.8455 | 94.2 |
| | | 2 | 37.4797 | |
| 50 mL | CR/AD | 1 | 9.9941 | 18.4 |
| | | 2 | 8.4407 | |
| | OxyContin | 1 | 47.3937 | 94.7 |
| | | 2 | 47.2864 | |

TABLE 42
Filtration Testing Results of CR/AD Tablet and
CR OxyContin Tablet Using Alcohol (40%) as the Solvent.

| Solvent | | | | Volume Recovery |
|---|---|---|---|---|
| Volume | Tablet | Trials | Volume (mL) | (%) |
| 10 mL | CR/AD | 1 | 0.8315 | 6.7 |
| | | 2 | 0.4995 | |
| | OxyContin | 1 | 7.4752 | 69.2 |
| | | 2 | 6.3674 | |
| 20 mL | CR/AD | 1 | 3.8763 | 16.0 |
| | | 2 | 2.5272 | |
| | OxyContin | 1 | 17.7416 | 86.9 |
| | | 2 | 17.0075 | |
| 30 mL | CR/AD | 1 | 7.7742 | 20.9 |
| | | 2 | 4.7497 | |
| | OxyContin | 1 | 27.5840 | 92.2 |
| | | 2 | 27.7572 | |
| 40 mL | CR/AD | 1 | 8.4718 | 15.6 |
| | | 2 | 4.0465 | |
| | OxyContin | 1 | 37.0257 | 92.9 |
| | | 2 | 37.3258 | |
| 50 mL | CR/AD | 1 | 10.7333 | 24.1 |
| | | 2 | 13.3577 | |
| | OxyContin | 1 | 46.8663 | 93.9 |
| | | 2 | 47.0582 | |

Surprisingly, the results indicate the CR/AD formulation is superior to OxyContin in preventing the filtration of an aqueous extract of the tablet when manually comminuted with water. Using 10 mL of water, the CR/AD tablet provided a volume recovery of 9.4% compared to OxyContin that had a volume recovery of about 9-fold greater, 85.8% (Table 41). Using 50 mL of water, the CR/AD tablet provided a volume recovery of 18.4% compared to OxyContin that had a volume recovery of about 5-fold greater, 94.7% (Table 41).

Also unexpected are the comparative results of CR/AD and OxyContin using alcohol as the extraction liquid. Using 10 mL of alcohol, the CR/AD tablet provided a volume recovery of 6.7% compared to OxyContin that had a volume recovery of about 10-fold greater, 69.2% (Table 42). Using 50 mL of alcohol, the CR/AD tablet provided a volume recovery of 24.1% compared to OxyContin that had a volume recovery of about 4-fold greater, 93.9% (Table 42).

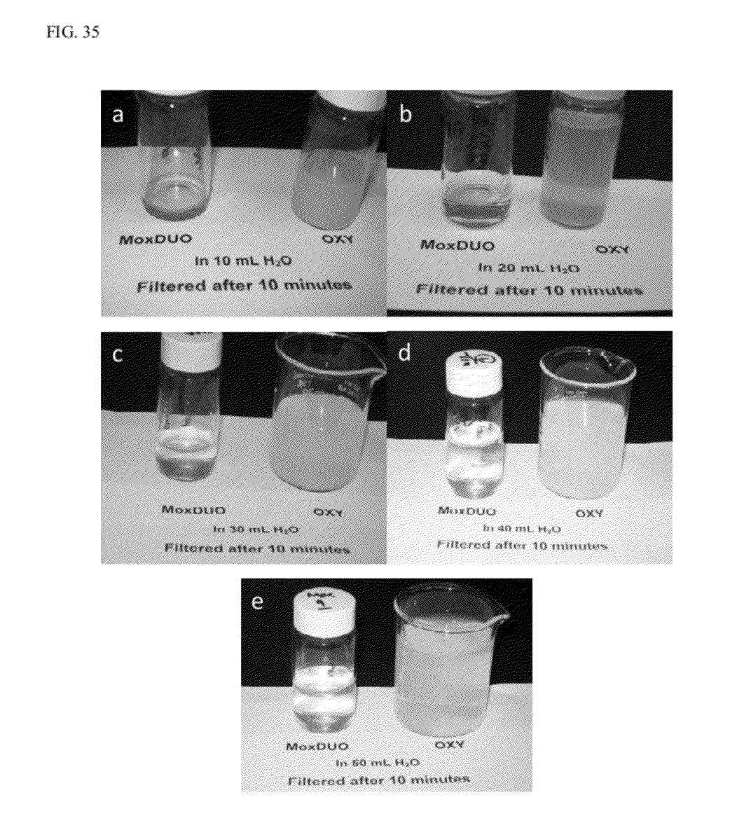

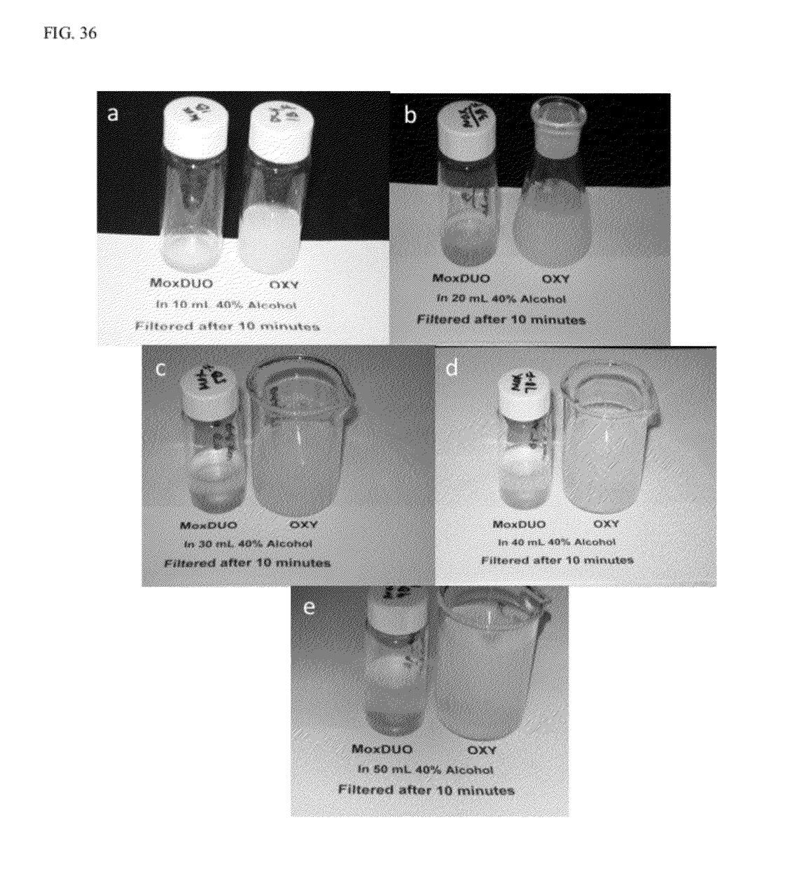

Notably, OxyContin filtration was not retarded in any significant manner, but the resulting filtrate was cloudy and possibly unsuitable for intravenous use, as shown in FIGS. 35 and 36.

The analysis of the actual quantity of opioids recovered in the filtrates shows that the CR/AD tablet was surprisingly superior to OxyContin. For example, using alcohol as the extraction liquid at 10 mL, the CR/AD tablets had a total % recovery of oxycodone of 42.9%, which is about 2-fold less than the % recovery of oxycodone from OxyContin, 90.5% (compare Tables 47 and 48). These results show the practical superiority of the abuse deterrent technology of the instant invention.

TABLE 43
Extraction Testing Results of Morphine (3 mg) from
CR/AD Tablet Using Water as the Solvent.

| Solvent | | Opioids in Filtrate | Opioids in Filtrate | Total Recovered |
|---|---|---|---|---|
| Volume | Trials | (mg/mL) | Average (mg/mL) | (%) |
| 10 mL | 1 | 1.71 | 1.69 | 56.4 |
| | 2 | 1.68 | | |
| 20 mL | 1 | 0.89 | 0.98 | 65.5 |
| | 2 | 1.07 | | |
| 30 mL | 1 | 0.65 | 0.61 | 61.3 |
| | 2 | 0.58 | | |
| 40 mL | 1 | 0.51 | 0.51 | 68.2 |
| | 2 | 0.51 | | |
| 50 mL | 1 | 0.38 | 0.39 | 65.1 |
| | 2 | 0.40 | | |

TABLE 44
Extraction Testing Results of Oxycodone (2 mg) from
CR/AD Tablet Using Water as the Solvent.

| Solvent | | Opioids in Filtrate | Opioids in Filtrate | Total Recovered |
|---|---|---|---|---|
| Volume | Trials | (mg/mL) | Average (mg/mL) | (%) |
| 10 mL | 1 | 1.18 | 1.17 | 58.6 |
| | 2 | 1.16 | | |
| 20 mL | 1 | 0.61 | 0.67 | 66.9 |
| | 2 | 0.73 | | |
| 30 mL | 1 | 0.44 | 0.41 | 62.0 |
| | 2 | 0.39 | | |
| 40 mL | 1 | 0.35 | 0.35 | 70.0 |
| | 2 | 0.35 | | |
| 50 mL | 1 | 0.27 | 0.27 | 67.8 |
| | 2 | 0.28 | | |

TABLE 45
Extraction Testing Results of Oxycodone (20 mg) from
OxyContin Tablet Using Water as the Solvent.

| Solvent | | Opioids in | Opioids in Filtrate | Total Recovered |
|---|---|---|---|---|
| Volume | Trials | Filtrate (mg/mL) | Average (mg/mL) | (%) |
| 10 mL | 1 | 1.71 | 1.73 | 86.3 |
| | 2 | 1.74 | | |
| 20 mL | 1 | 0.86 | 0.88 | 88.0 |
| | 2 | 0.90 | | |
| 30 mL | 1 | 0.59 | 0.59 | 88.1 |
| | 2 | 0.58 | | |
| 40 mL | 1 | 0.45 | 0.44 | 88.8 |
| | 2 | 0.44 | | |
| 50 mL | 1 | 0.34 | 0.34 | 85.5 |
| | 2 | 0.34 | | |

Alcohol extraction is expected to provide a more efficient recovery from an extraction process. Surprisingly, the CR/AD tablet is more effective in preventing full recovery of the available active ingredients in alcohol as compared to water (compare Tables 43 and 46, and Tables 44 and 47).

With regards to morphine, particular only to the CR/AD tablet, attempted extraction using the lowest volume of alcohol only resulted in 36% recovery of the available morphine present in the tablet as shown in Table 46.

TABLE 46
Extraction Testing Results of Morphine (3 mg) from
CR/AD Tablet Using 40% Alcohol as the Solvent.

| Solvent | | Opioids in Filtrate | Opioids in Filtrate | Total Recovered |
|---|---|---|---|---|
| Volume | Trials | (mg/mL) | Average (mg/mL) | (%) |
| 10 mL | 1 | 0.72 | 1.08 | 36.1 |
| | 2 | 1.44 | | |
| 20 mL | 1 | 0.84 | 0.81 | 53.9 |
| | 2 | 0.78 | | |
| 30 mL | 1 | 0.60 | 0.62 | 61.5 |
| | 2 | 0.63 | | |
| 40 mL | 1 | 0.47 | 0.44 | 58.7 |
| | 2 | 0.41 | | |
| 50 mL | 1 | 0.44 | 0.41 | 68.7 |
| | 2 | 0.39 | | |

TABLE 47
Extraction Testing Results of Oxycodone (2 mg) from
CR/AD Tablet Using 40% Alcohol as the Solvent.

| Solvent | | Opioids in | Opioids in Filtrate | Total Recovered |
|---|---|---|---|---|
| Volume | Trials | Filtrate(mg/mL) | Average (mg/mL) | (%) |
| 10 mL | 1 | 0.60 | 0.86 | 42.9 |
| | 2 | 1.11 | | |
| 20 mL | 1 | 0.63 | 0.62 | 61.5 |
| | 2 | 0.60 | | |
| 30 mL | 1 | 0.45 | 0.46 | 68.6 |
| | 2 | 0.46 | | |
| 40 mL | 1 | 0.35 | 0.33 | 65.3 |
| | 2 | 0.30 | | |
| 50 mL | 1 | 0.31 | 0.29 | 73.4 |
| | 2 | 0.28 | | |

TABLE 48
Extraction Testing Results of Oxycodone (20 mg) from
OxyContin Tablet Using 40% Alcohol as the Solvent.

| Solvent | | Opioids in Filtrate | Opioids in Filtrate | Total Recovered |
|---|---|---|---|---|
| Volume | Trials | (mg/mL) | Average (mg/mL) | (%) |
| 10 mL | 1 | 1.85 | 1.81 | 90.5 |
| | 2 | 1.77 | | |
| 20 mL | 1 | 0.89 | 0.92 | 92.1 |
| | 2 | 0.96 | | |
| 30 mL | 1 | 0.59 | 0.60 | 90.1 |
| | 2 | 0.62 | | |
| 40 mL | 1 | 0.44 | 0.44 | 87.7 |
| | 2 | 0.44 | | |
| 50 mL | 1 | 0.35 | 0.35 | 88.1 |
| | 2 | 0.35 | | |

The ease of opioid extraction from a whole dosage unit in the presence of 95% and 40% alcohol was investigated for the CR/AD and OxyContin tablet formulations. The whole dosage unit was pre-soaked with 20.0 mL of 95% v/v ethanol, 40% v/v ethanol, or 0.1N HCl (simulating gastric fluid). The solution was stirred at a slow speed for 30 minutes, and then 15.0 mL of either 95% v/v ethanol (for when 95% v/v ethanol or 0.1N HCl was used in the pre-soak) or 40 v/v ethanol (for when 40% v/v was used in the pre-soak) was added and stirred slowly with the solution. The resulting stock solution continued to be stirred, and 1 mL samples were removed immediately and after 10, 20, 30, 40, and 60 minutes to be filtered and then assessed using high-performance liquid chromatography for concentrations of morphine sulphate and oxycodone HCl.

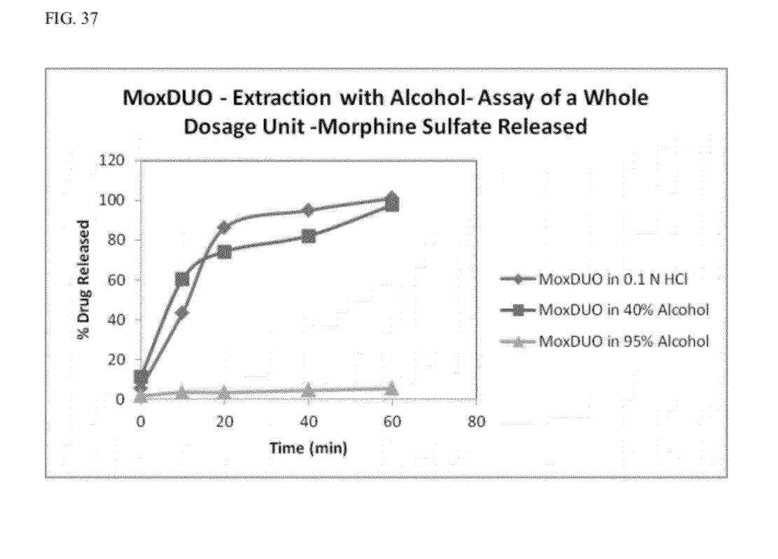

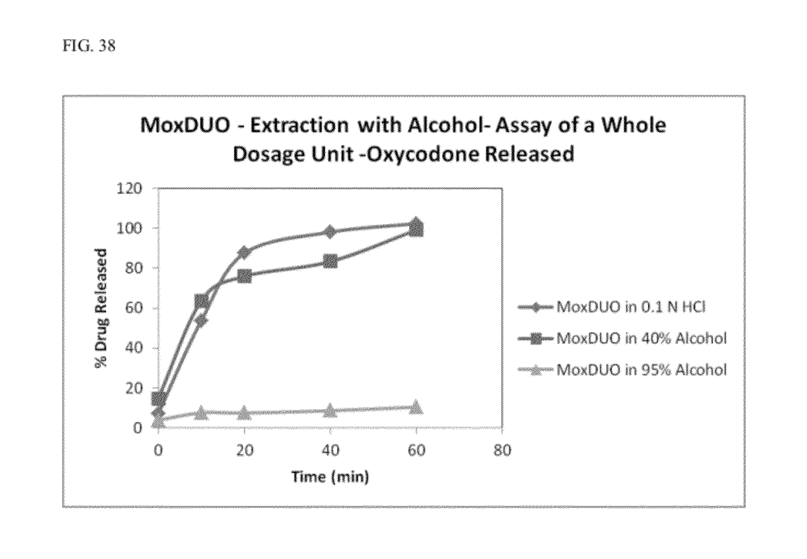

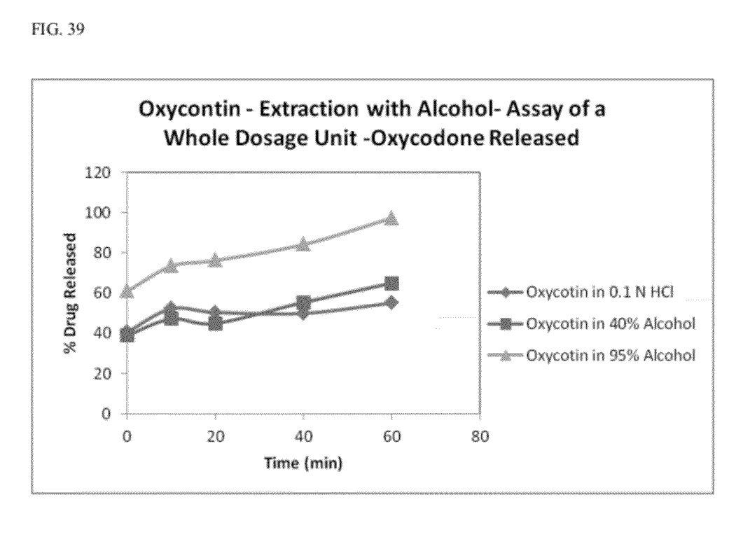

As shown in FIGS. 37-39, the use of pure alcohol (95%) has little effect on the CR/AD tablet formulation whereas the OxyContin tablet formulation readily discharged 60% of the available oxycodone dose upon 30 minutes of contact with the solvent. Similarly, the use of 40% alcohol, simulating straight vodka, performed in the same manner with the OxyContin tablet formulation, while the CR/AD tablet displayed a time-dependent resistance to extraction of the opioids, only reaching appreciable levels after 20 minutes post soaking. This characteristic was also duplicated when 0.1N HCl was used in the pre-soak conditions.

It should be understood, of course, that the foregoing relates only to certain disclosed embodiments of the present invention and that numerous modifications or alterations may be made therein without departing from the spirit and scope of the invention as set forth in the appended claims.

What is claimed is:

1. An abuse resistant pharmaceutical formulation comprising:
(a) one or more abusable drugs, and
(b) one or more abuse deterrent components, wherein each abuse deterrent component comprises:
(i) a core comprising one or more materials that are both hydrophilic and hydrophobic; and
(ii) optionally a coating;
wherein the one or more abuse deterrent components slows, reduces, or slows and reduces, extraction by aqueous or alcoholic liquids of the one or more abusable drugs from the formulation.

2. The abuse resistant pharmaceutical formulation of claim 1, wherein the one or more abusable drugs comprises one or more water soluble abusable drugs.

3. The abuse resistant pharmaceutical formulation of claim 1, wherein the one or more abusable drugs comprises one or more opioids.

4. The abuse resistant pharmaceutical formulation of claim 3, wherein the one or more opioids comprises morphine and oxycodone.

5. The abuse resistant formulation of claim 1, wherein the one or more abuse deterrent components is in a form selected from the group consisting of pellets, beads, beadlets, granules, powder, or a combination thereof.

6. The abuse resistant formulation of claim 1, wherein the one or more abuse deterrent components is in a ratio to the rest of the formulation of between about 1:1 w/w and about 1:5 w/w.

7. The abuse resistant formulation of claim 1, wherein the one or more abuse deterrent components is in a ratio to the one or more abusable drugs of between about 1:1 w/w and about 1:10 w/w.

8. The abuse resistant pharmaceutical formulation of claim 1, wherein the material that is both hydrophilic and hydrophobic is selected from the group consisting of polyacrylic acid, acrylic acid cross-linked with allyl ethers of polyalcohols, hydroxypropyl cellulose, hydroxypropyl methylcellulose:hydroxypropyl cellulose mixture, polyvinylpyrrolidone, polyethylene oxide, methylcellulose, xanthan gum, guar gum, polyethylene glycol, methacrylic acid copolymer, colloidal silicon dioxide, cellulose gum, starch, sodium starch glycolate, sodium alginate, and combinations thereof.

9. The abuse resistant pharmaceutical formulation of claim 8, wherein the material that is both hydrophilic and hydrophobic is acrylic acid cross-linked with allyl ethers of polyalcohols.

10. The abuse resistant pharmaceutical formulation of claim 9, wherein the acrylic acid cross-linked with allyl ethers of polyalcohols is a carbomer.

11. The abuse resistant pharmaceutical formulation of claim 1, wherein the pH-sensitive coating comprises methacrylic acid copolymer dispersion, hypromellose acetate succinate, and cellulose acetate phthalate.

12. The abuse resistant pharmaceutical formulation of claim 1, further comprising an alkalizing agent.

13. The abuse resistant pharmaceutical formulation of claim 12, wherein the alkalizing agent is selected from the group consisting of polyplasdone XL, talc, meglumine, NaHCO₃, and polyvinylpyrrolidone.

14. The abuse resistant pharmaceutical formulation of claim 13, wherein the alkalizing agent is meglumine.

15. The abuse resistant pharmaceutical formulation of claim 12, wherein the alkalizing agents is in a form selected from the group consisting of pellets, beads, beadlets, granules, powder, or a combination thereof.

16. The abuse resistant pharmaceutical formulation of claim 11, wherein the one or more alkalizing agents is in a ratio to the one or more abuse deterrent components of about 40:60 w/w to about 80:20 w/w.

17. The abuse resistant pharmaceutical formulation of claim 1 further comprising a plasticizer.

18. The abuse resistant pharmaceutical formulation of claim 1, wherein the formulation is immediate release, controlled release, or a combination thereof.

19. A method of reducing the amount of abusable drug that can be extracted by aqueous or alcoholic liquids from a pharmaceutical formulation that comprises said abusable drug, wherein the method comprises admixing the abusable drug with one or more abuse deterrent components, each abuse deterrent component comprising (a) a core comprising one or more materials that are both hydrophilic and hydrophobic; and (b) optionally a coating.

20. A method of reducing the rate at which an abusable drug can be extracted by aqueous or alcoholic liquids from a pharmaceutical formulation that comprises said abusable drug, wherein the method comprises admixing the abusable drug with one or more abuse deterrent components, each abuse deterrent component comprising: (a) a core comprising one or more materials that are both hydrophilic and hydrophobic; and (b) optionally a coating.
